(19) United States
(12) Patent Application Publication
(10) Pub. No.: US 2017/0073430 A1
(43) Pub. Date: Mar. 16, 2017

(54) NOVEL ANTI-RNF43 ANTIBODIES AND METHODS OF USE

(71) Applicant: ABBVIE STEMCENTRX LLC, North Chicago, IL (US)

(72) Inventors: MANDY BOONTANRART, San Francisco, CA (US); DEEPTI ROKKAM, Sunnyvale, CA (US); DAVID LIU, SAN FRANCISCO, CA (US); SCOTT J. DYLLA, Emerald Hills, CA (US); MONETTE AUJAY, San Francisco, CA (US)

(73) Assignee: AbbVie Stemcentrx LLC, North Chicago, IL (US)

(21) Appl. No.: 15/305,861

(22) PCT Filed: Apr. 21, 2015
(86) PCT No.: PCT/US15/26904

§ 371 Date: Oct. 21, 2016

Related U.S. Application Data
(60) Provisional application No. 61/982,294, filed on Apr. 21, 2014.

(51) Int. Cl. C07K 16/40; A61K 47/48

(52) U.S. Cl. CPC C07K 16/40; A61K 47/48646; C07K 2317/77; C07K 2317/76; C07K 2317/33; C07K 2317/24; C07K 2317/565

(57) ABSTRACT

The invention discloses novel anti-RNF43 antibodies and derivatives thereof, including antibody drug conjugates, and methods of using such anti-RNF43 antibodies and antibody drug conjugates to diagnose and treat cancer.

### CROSS REFERENCED APPLICATIONS

This application claims the benefit of U.S. Provisional Application No. 61/982,294 filed on 21 Apr. 2014, which is incorporated herein by reference in its entirety.

### SEQUENCE LISTING

This application contains a sequence listing which has been submitted in ASCII format via EFS-Web and is hereby incorporated by reference in its entirety. Said ASCII copy, created on Apr. 21, 2015, is named sc3701pct_569697_1210 WO_SEQL_042115.txt and is 278,070 (271 KB) in size.

### FIELD OF THE INVENTION

This application generally relates to novel anti-RNF43 antibodies or immunoreactive fragments thereof and compositions, including antibody drug conjugates, comprising the same for the treatment, diagnosis or prophylaxis of cancer and any recurrence or metastasis thereof. Selected embodiments of the invention provide for the use of such anti-RNF43 antibodies or antibody drug conjugates for the treatment of cancer comprising a reduction in tumorigenic cell frequency.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rates for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

The invention is generally directed towards antibodies, antibody drug conjugates (ADCs) and pharmaceutical compositions that may be used in the prophylaxis, diagnosis or treatment of cancer. In certain embodiments the invention comprises an antibody drug conjugate of the formula M-[L-D]n, or a pharmaceutically acceptable salt thereof, wherein M comprises an anti-RNF43 antibody; L comprises a linker; D comprises a cytotoxin; and n is an integer from 1 to 20.

In another embodiment, the anti-RNF43 ADCs of the invention comprise an anti-RNF43 antibody that is an internalizing antibody. In another aspect, the invention is directed to an anti-RNF43 antibody that is an internalizing antibody.

In further embodiments the anti-RNF43 ADCs of the invention comprise an anti-RNF43 antibody that is a chimeric, CDR grafted or humanized antibody, or fragment thereof. In a further aspect, the invention is directed to anti-RNF43 antibodies that are chimeric, CDR grafted or humanized.

In one aspect of the invention, the anti-RNF43 ADCs of the invention comprise an anti-RNF43 antibody that binds to tumor initiating cells. In another aspect, the invention is directed to anti-RNF43 antibodies that bind to tumor initiating cells.

The invention also comprises anti-RNF43 ADCs comprising an anti-RNF43 antibody that binds to human RNF43 (SEQ ID NO: 5) and does not bind to human ZNRF3 (SEQ ID NO: 6). In another aspect, the invention is directed to anti-RNF43 antibodies that bind to human RNF43 (SEQ ID NO: 5) and do not bind to human ZNRF3 (SEQ ID NO: 6).

RNF43 has been shown to be a negative feedback regulator of the WNT signaling pathway and therefore antibodies that bind RNF43 may have the ability to interfere with RNF43 function. As shown herein, there are three main categories of anti-RNF43 antibodies: those that are “neutral antibodies” with respect to WNT signaling and do not affect the WNT signaling pathway, those that increase WNT signaling and those that decrease WNT signaling. Thus in one aspect, the invention is directed to an anti-RNF43 ADC comprising an anti-RNF43 antibody that binds to human RNF43 (SEQ ID NO: 5) on the surface of a eukaryotic cell wherein the binding of the antibody decreases WNT signaling. In a further aspect, the invention is directed to an anti-RNF43 ADC comprising an anti-RNF43 antibody that binds to human RNF43 (SEQ ID NO: 5) on the surface of a eukaryotic cell wherein the binding of the antibody increases WNT signaling. In yet another aspect, the invention is directed to an anti-RNF43 ADC comprising an anti-RNF43 antibody that binds to human RNF43 (SEQ ID NO: 5) on the surface of a eukaryotic cell wherein the binding of the antibody does not affect WNT signaling. Thus, in some aspects, the anti-RNF43 ADCs of the invention will comprise anti-RNF43 antibodies that are “neutral antibodies” with respect to WNT signaling. In further aspects, the invention is directed to anti-RNF43 antibodies that bind to human RNF43 (SEQ ID NO: 5) and either increase, decrease or do not affect WNT signaling.

R-spondin (RSPO) is a protein involved in the WNT signaling pathway and blocks RNF43, thus leading to upregulation of WNT ligand production and an increase in WNT signaling. In one embodiment, the anti-RNF43 ADCs of the invention comprise an anti-RNF43 antibody that binds to human RNF43 (SEQ ID NO: 5) and blocks binding of R-spondin to RNF43. In another aspect, the invention is directed to anti-RNF43 antibodies that bind to human RNF43 (SEQ ID NO: 5) and do not block binding of R-spondin to RNF43.

In another aspect of the invention, the anti-RNF43 ADCs of the invention comprise an anti-RNF43 antibody that binds to human RNF43 (SEQ ID NO: 5) on the surface of a eukaryotic cell wherein the binding of the antibody does not block R-spondin-stimulated WNT signaling. In a further embodiment the anti-RNF43 ADCs of the invention comprise an anti-RNF43 antibody that binds to human RNF43 (SEQ ID NO: 5) on the surface of a eukaryotic cell wherein the binding of the antibody blocks R-spondin-stimulated WNT signaling.

In one embodiment, invention is directed to an isolated antibody that binds to human RNF43 (SEQ ID NO: 5) and competes for binding to human RNF43 with an antibody comprising: (1) a light chain variable region set forth as SEQ ID NO: 78 and a heavy chain variable region set forth as SEQ ID NO: 80; or (2) a light chain variable region set forth as SEQ ID NO: 110 and a heavy chain variable region set forth as SEQ ID NO: 112.

In another embodiment, the invention is directed to an isolated antibody that binds to human RNF43 (SEQ ID NO: 5) comprising a light chain comprising the following light chain complementarity determining regions (CDRL): CDRL1: SEQ ID NO: 288; CDRL2: SEQ ID NO: 289; CDRL3: SEQ ID NO: 290; and a heavy chain comprising the following heavy chain complementarity determining regions (CDRH): CDRH1: SEQ ID NO: 291; CDRH2: SEQ ID NO: 292; CDRH3: SEQ ID NO: 293.

In a further embodiment, the invention is directed to an isolated antibody that binds to human RNF43 (SEQ ID NO: 5) comprising a light chain comprising the following light chain complementarity determining regions (CDRL): CDRL1: SEQ ID NO: 294; CDRL2: SEQ ID NO: 295; CDRL3: SEQ ID NO: 296; and a heavy chain comprising the following CDRH: CDRH1: SEQ ID NO: 297; CDRH2: SEQ ID NO: 298; CDRH3: SEQ ID NO: 299.

One aspect of the invention is a humanized antibody that binds to human RNF43 (SEQ ID NO: 5) comprising a light chain set forth as SEQ ID NO: 273; and a heavy chain set forth as SEQ ID NO: 275.

Another aspect of the invention is a humanized antibody that binds to human RNF43 (SEQ ID NO: 5) comprising a light chain set forth as SEQ ID NO: 276; and a heavy chain set forth as SEQ ID NO: 278.

A further aspect of the invention is a nucleic acid encoding a light chain set forth as SEQ ID NO: 273 or 276, or a heavy chain set forth as SEQ ID NO: 275 or 278. In another aspect, the invention is a host cell comprising a vector comprising the above nucleic acid.

In another aspect, the invention is directed to a pharmaceutical composition comprising any anti-RNF43 antibody or ADC described herein.

In one embodiment, the invention is directed to a method of treating cancer comprising administering a pharmaceutical composition of invention to a subject in need thereof. In some aspects, the cancer is selected from colorectal cancer or lung cancer.

### BRIEF DESCRIPTION OF THE FIGURES

FIG. 1A depicts expression levels of RNF43 as measured using whole transcriptome (SOLiD) sequencing of RNA derived from normal tissue and patient derived xenograft (PDX) tumor cells.

FIG. 1B shows expression levels of RNF43 as measured using whole transcriptome (Illumina) sequencing of RNA derived from normal tissue and patient derived xenograft (PDX) tumor cells.

FIG. 2A depicts the relative expression levels of RNF43 transcripts as measured by qRT-PCR in RNA samples isolated from normal tissue and from a variety of PDX tumors.

FIG. 2B depicts the relative expression levels of RNF43 transcripts as measured by qRT-PCR in RNA samples isolated from various normal tissues and from cancer stem cells (CSC) and non-tumorigenic (NTG) cells isolated from a variety of PDX tumors.

FIG. 3 shows the normalized intensity value of RNF43 transcript expression measured by microarray hybridization in normal tissues and a variety of PDX cell lines.

FIG. 4 shows expression of RNF43 transcripts in normal tissues and primary tumors from The Cancer Genome Atlas (TCGA), a publically available dataset.

FIG. 5A shows various physiological and functional characteristics of exemplary anti-RNF43 antibodies.

FIG. 5B shows an alignment of the extracellular domains of RNF43 (SEQ ID NO: 3) and ZNRF3 (SEQ ID NO: 4).

FIG. 6A shows a schematic of the genetic interactions in a simplified version of the canonical WNT signaling pathway.

FIG. 6B shows the behavior of a pair of canonical WNT signaling reporter cell lines, with or without overexpression of RNF43, in response to treatment with conditioned medium containing or lacking WNT3A.

FIGS. 7A and 7B provide contiguous amino acid sequences (SEQ ID NOS: 22-268, even numbers) of light and heavy chain variable regions of exemplary murine and humanized anti-RNF43 antibodies.

FIG. 7C provides the nucleic acid sequences (SEQ ID NOS: 21-269, odd numbers) encoding the amino acid sequences of the anti-RNF43 antibodies in FIGS. 7A and 7B.

FIG. 7D provides amino acid sequences for the full length humanized antibodies hSC37.2, hSC37.17, hSC37.17ss1, hSC37.39, hSC37.39ss1, hSC37.67 and hSC37.67variant 1.

FIGS. 7E to 7H show annotated amino acid sequences (numbered as per Kabat et al.) of the light and heavy chain variable regions of mouse anti-RNF43 antibodies, SC37.2 (FIG. 7E), SC37.17 (FIG. 7F), SC37.39 (FIG. 7G), and SC37.67 (FIG. 7H), wherein the CDRs are derived using Kabat, Chothia, ABM and Contact methodology.

FIG. 8 shows the relative protein expression of human RNF43 measured using an electrochemiluminescent sandwich ELISA assay.

FIG. 9 shows RNA expression of RNF43 in various PDX tumor cell lines determined by in situ hybridization.

FIG. 10 shows surface protein expression of RNF43 (black line) in representative PDX cell lines determined by flow cytometry compared to an isotype-control stained population (solid gray) in cancer stem cells (CSC) (solid black line) or non-tumorigenic cells (NTG) (dotted black line). Mean Fluorescence Intensity (MFI) values are shown for the IgG1 isotype control antibody and the anti-RNF43 antibodies in respect of each PDX line tested.

FIG. 11 shows the ability of selected anti-RNF43 humanized antibodies (combined with goat anti human directly conjugated to saporin) to internalize into HEK293T cells overexpressing RNF43 protein and to kill such cells.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

RNF43 has surprisingly been found to be a biological marker of a number of tumor types and this association may be exploited for the treatment of such tumors. It has also unexpectedly been found that RNF43 is associated with tumorigenic cells and may be effectively exploited to inhibit or eliminate them. Tumorigenic cells, which will be described in more detail below, are known to exhibit resistance to many conventional treatments. In contrast to the teachings of the prior art, the disclosed compounds and methods effectively overcome this inherent resistance.

The invention provides anti-RNF43 antibodies (including antibody drug conjugates) and their use in the prognosis, diagnosis, theragnosis, treatment and/or prevention of a variety of RNF43-associated cancers regardless of any particular mechanism of action or specifically targeted cellular or molecular component.

### I. RNF43 PHYSIOLOGY

RING finger protein 43 (RNF43; also known as E3 ubiquitin-protein ligase RNF43, or RNF124) is a single-pass type 1 transmembrane protein that functions as an important feedback regulator of WNT signaling. Representative RNF43 protein orthologs include, but are not limited to, human (NP_060233), chimpanzee (XP_001172611), rhesus monkey (XP_001106574), rat (NP_001129393), and mouse (NP_766036). In humans, the RNF43 gene consists of 10 exons spanning approximately 63.9 kBp on chromosome 17, at cytogenetic location 17q22. Transcription of the human RNF43 locus yields a spliced 4.6 kBp mature mRNA transcript (NM_017763), encoding a 783 amino acid preprotein (NP_060233). Processing of the RNF43 preprotein is predicted to involve the removal of the first 23 amino acids comprising the secretion signal peptide. The mature RNF43 protein is predicted to contain 174 amino acids in the extracellular domain (amino acids 24-197), a 21 amino acid helical transmembrane domain (amino acids 198-218), and a 565 amino acid cytoplasmic domain (amino acids 219-783), a portion of which comprises the atypical RING domain zinc finger (amino acids 272-313) from which the protein derives its name. RING domains are sequence defined domains linked to the formation of zinc finger structures mediating protein-protein interactions, and are commonly found in proteins that participate in protein ubiquitylation processes.

Ubiquitylation of a protein is a biological conjugation process in which ubiquitin (Ub), a heat stable 76 amino acid protein, is covalently attached to various lysine residues in a target protein. Ub is added via its C-terminal glycine residue (UbG76) to an epsilon-amino group of lysine in the targeted protein (for an overview, see Shen et al., 2013; PMID 23822887). In addition, because Ub itself contains seven lysines (UbK6, UbK11, UbK27, UbK29, UbK33, UbK49 and UbK63), target proteins can be polyubiquitylated via concatenation of Ub moieties to one another after the initial mono-ubiquitylation of the target protein at a given lysine. Cells utilize Ub tags as signals for how to traffic the ubiquitylated protein, depending upon the nature of the Ub covalent linkage and multimeric state of the Ub tag. For instance, targeting of misfolded, oxidized or short-lived proteins to the 26S proteasome, the so called ubiquitin-proteasome system, occurs when a protein is tagged by polyUb chains containing UbG76-UbK48 linkages (Dikic et al, 2009; PMID: 19773779). In contrast, multiple monoubiquitylation may direct cell surface proteins such as receptor tyrosine kinases or serpentine receptors through various endocytic compartments ultimately leading to degradation in the lysosome (Railborg and Stenmark, 2009, PMID: 19325624; Mukai et al., 2010, PMID: 20495530; Haglund and Dikic, 2012; PMID: 22357968).

The biological conjugation of Ub to target proteins is mediated by an enzymatic cascade that involves three distinct enzymes: Ub activating enzymes (E1), which chemically activate the UbG76; Ub conjugating enzymes (E2), which act as carriers of the activated Ub; and Ub protein-ligases (E3), which complex with both E2 proteins and the target protein and mediate transfer of the activated Ub from E2 to the protein target. Within the human genome, there are hundreds of E3 Ub-protein ligases that confer specificity to the process, with each E3 protein recognizing specific or limited sets of proteins. RING finger E3 proteins are the most abundant type of E3 Ub-protein ligase, and function as scaffolds to bring the protein substrate in proximity to the activated E2-Ub complex. RNF43 was identified as a probable E3 ubiquitin-protein ligase based upon the presence of a conserved RING sequence (Yagyu et al., 2004, PMID: 15492824), a result confirmed by subsequent studies in which it was shown that overexpression of RNF43 promoted ubiquitin-mediated down-regulation of various cell surface molecules (Koo et al., 2012, PMID: 22895187).

Proper expression and function of E3 Ub-protein ligases is likely essential to the trafficking, function and regulated degradation of proteins involved in diverse biological processes (Haglund and Dikic, supra). However, disregulated expression or misfunction of E3 ubiquitin-protein ligases may contribute to the development of cancer (for overviews, see Mani and Gelmann, 2005, PMID: 16034054; Hoeller and Dikic, 2009, PMID: 19325623; Nakayama and Nakayama, 2006, PMID: 16633365). RNF43 was identified by expression profiling as being upregulated in colorectal cancer, wherein these authors also reported limited expression in fetal kidney and lung, with undetectable expression in normal adult tissues as measured by RNA blotting (Yagyu et al., 2004, PMID: 15492824). Some initial studies reported detection of the protein in the endoplasmic reticulum and nucleus (Sugiura et al., 2008, PMID: 18313049) or as a secreted protein (Yagyu et al., 2004, PMID: 15492824). However, more recent studies have placed RNF43 at the cell surface, linked its function to modulation of WNT signaling, and suggested that proper cell surface localization is required for its functional activity in modulation of WNT signaling (Hao et al., 2012, PMID: 22575959; Koo et al., 2012, PMID: 22895187; Jiang et al., 2013, PMID: 23847203; Tsukiyama et al., 2015, PMID: 25825523).

1. The Role of RNF43 in WNT Signaling

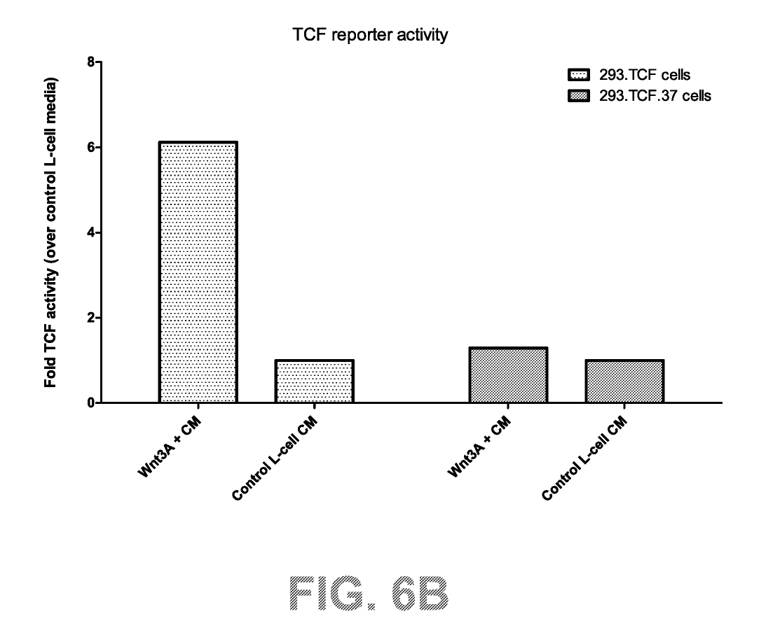

The WNT pathway is a critical developmental and stem cell-associated signaling pathway regulating cell growth and differentiation (Seifert and Mlodzik, 2007, PMID: 17230199; van der Flier and Clevers, 2009, PMID: 18808327; Nihers, 2012, PMID: 23151663), and one whose aberrant reactivation or overactivation has been linked to cancer (Barker and Clevers, 2006, PMID: 17139285; Krausova and Korinek, 2013, PMID: 24308963). In the human genome, there are 19 different WNT genes encoding 19 WNT protein ligands that bind to various cell surface receptors to form ligand/receptor complexes, transmitting a signal from the outside of the cell to the inside of the cell via a pathway of specific protein-protein interactions known as WNT signaling pathways. There are three well characterized WNT signaling pathways: (1) the canonical WNT signaling pathway, (2) the noncanonical planar cell polarity pathway, and (3) the noncanonical WNT/calcium pathway. While all three WNT signaling pathways are activated by binding of a WNT protein ligand to a Frizzed (FZD) protein receptor at the surface of the cell with subsequent signaling to Dishevelled (DVL) proteins on the inside of the cell membrane, the canonical pathway operates through the binding of WNT to FZD and a low density lipoprotein receptor related protein 5 or 6 (LRP5/6) co-receptor resulting in downstream protein interactions that lead to the stabilization of the transcriptional coactivator protein beta-catenin (CTNNB1). Stabilized beta-catenin is able to translocate to the nucleus, partner with TCF/LEF proteins, and activate transcription of WNT target genes that promote cell growth and differentiation, as well as negative feedback regulators of the signaling pathway. A simplified map of the canonical WNT pathway is shown in FIG. 6A. In contrast, the non-canonical planar cell polarity pathway does not proceed via stabilization of a beta-catenin intermediate; instead, the DVL protein regulates the activity of alternative cell surface co-receptors such as protein tyrosine kinase 7 (PTK7) or van Gogh-like proteins (VANGL1, VANGL2) resulting in modulation of the behavior of actin and the cell cytoskeleton. Similarly, the non-canonical WNT/calcium signaling pathway does not proceed via a stabilized beta-catenin intermediate, but instead results when signaling from DVL proteins and associated G-proteins modulate intracellular calcium levels, which ultimately leads to changes in cell adhesion, migration, and tissue separation.

E3 Ub-protein ligases RNF43 and ZNRF3 have been shown to be important regulators of WNT signaling. Both of these functionally homologous yet sequence divergent proteins (identity only 26% overall, 40% in ectodomains and 69% in the atypical RING domain zinc fingers) are negative feedback regulators of WNT signaling, which can be inferred by their positive correlation with the expression of AXIN2 (a known WNT response gene that also acts as a negative feedback regulator of WNT signaling) (Lustig et al., 2002, PMID: 11809809), by their elevated expression in primary colorectal tumors with hyperactive β-catenin signaling, and by their reduced expression in cells treated with siRNA against β-catenin (Hao et al., supra). Recently, two WNT responsive elements were reported to be located in an intron of the RNF43 gene, directly linking β-catenin signaling to RNF43 upregulation (Takahaski et al., 2014, PMID: 24466159). RNF43 and ZNRF3 each modulate cell surface FZD and LRP receptor levels by controlling the ubiquitylation of these receptors (Koo et al., supra; Hao et al., supra). In the case of RNF43, both the ectodomain and functional RING domains are required for this ability to ubiquitinylate FZDs and modulate canonical WNT signaling. The biological function of RNF43 in fine-tuning canonical WNT signaling was further shown in a series of studies that demonstrated that R-spondin proteins, through their physical association with E3 ubiquitin-protein ligases, suppressed the levels of these E3 ubiquitin-protein ligases resulting in elevated WNT receptor expression at the cell surface and in consequence positively potentiating WNT signaling (Chen et al., 2013, PMID: 23756651; Hao et al., supra). Mutations which functionally inactivate RNF43 in pancreatic ductal adenocarcinoma conferred WNT dependency upon these tumors (Jiang et al., supra). Finally, the association of disregulated RNF43 expression and its effects upon WNT signaling in stem cells and in cancer was demonstrated by Koo et al. (supra). RNF43 and ZNRF3 expression was found to be restricted to the LGR5⁺ stem cells compartment in the intestines of mice. Mice with an intestinal double knock-out of the RNF43 and ZNRF3 genes developed rapidly growing adenomas with phenotypes consistent with hyperactivation of β-catenin signaling, and a morphology consistent with Paneth cell and intestinal stem cell hyperplasia. It has also been recently shown that RNF43 may also modulate non-canonical WNT signaling via its interactions with DVL proteins (Tsukiyama et al., 2015, PMID: 2582552).

The above studies demonstrate that RNF43 is a node in an elaborate agonist, antagonist, and anti-antagonist feedback network for WNT signaling, with possible implications for the development of cancer. β-catenin hyperactivation is a common attribute of numerous hyperplasias and cancers, thus these cancers may show elevated RNF43 expression as part of a failed homostatic response to β-catenin hyperactivation.

2. Measurement of WNT Signaling

The function of protein modulators of the canonical WNT signaling pathway can be elucidated using assays that: (1) either directly assess elevated transcription of “WNT responsive” genes (e.g, AXIN2, MYC, CCND1, ASCL2) that naturally contain DNA binding sites (also termed WNT response elements, or WREs) for TCF/LEF transcription factors, or (2) by using synthetic reporter gene constructs, in which binding of the TCF/LEF factors to WREs leads to transcription, subsequent translation, and therefore activity of the reporter gene product (e.g., GFP, luciferase, or reporter enzymes whose activity can easily be measured). In one embodiment, WNT activity can be measured using the TOPFLASH assay or its derivatives (Korinek et al., 1997, PMID: 9065401; Veeman et al., 2003, PMID: 12699626). In another embodiment it is possible to use a WNT responsive reporter cell line that contains all the proteins required for the WNT signaling pathway and has been engineered to express a reporter gene, for example the firefly luciferase gene, under control of DNA sequences that contain WRE. In the present invention, a WNT responsive reporter cell line, termed 293.TCF, was generated and used to determine the ability of the anti-RNF43 antibodies or antibody drug conjugates of the invention to modulate canonical WNT signaling (see Example 8). The 293.TCF cell line expresses the firefly luciferase gene downstream of four WREs. In the presence of a WNT ligand (e.g., WNT3A) or alternative “WNT activators”, TCF/LEF transcription factors bind the WREs, and activate transcription of the firefly luciferase gene, ultimately resulting in an increase in luciferase enzyme activity (e.g., the production of light) as measured when appropriate substrate and cofactors for luciferase are added. As used herein, the term “WNT activator” means a compound (e.g. a WNT ligand) that activates the WNT signaling cascade. WNT activators can be identified, for example, using a WNT responsive reporter cell line (e,g, 293.TCF cells) either by simply adding a WNT activator compound (e.g. WNT3A) and observing whether there is an increase in luciferase activity compared to 293.TCF cells that are not exposed to such a WNT activator compound; or, e.g., by introducing the agent into the WNT responsive reporter cells using a variety of physiochemical techniques (e.g., lipofection, electroporation, etc.), or by transfection of DNA constructs encoding the compound (e.g., membrane-bound or transmembrane proteins) into the WNT-responsive reporter cell line and allowing the native machinery of the cells to produce the compound. In one embodiment, the 293.TCF cell line can be treated with supernatants from cells over-expressing WNT3A (e.g. conditioned medium from L/WNT3A cells), and the luciferase activity (i.e. WNT signaling) can be measured in WNT3A-treated cells compared to cells that are not treated with WNT3A (e.g. cells exposed to conditioned medium from parental L-cells that do not express WNT3A).

In contrast to WNT activators, various compounds, described herein as “WNT modulators” (e.g., R-spondins; FZD) are able to affect the activity of the WNT signaling pathway by increasing or decreasing WNT signaling in the presence of a WNT activator (e.g, WNT3A or conditioned medium from L/WNT3A cells), but are unable to activate the WNT signaling pathway independently of a WNT activator. WNT modulators can be identified, for example, using a WNT responsive reporter cell line (e,g, 293.TCF) by exposing these cells to a potential WNT modulator in conjunction with a WNT activator compound (e.g. WNT3A) and observing whether there is a measurable and significant change in luciferase activity compared to 293.TCF cells that are exposed to a WNT activator alone. Other methods for identifying WNT modulators include introducing the potential WNT modulator agent into the WNT responsive reporter cells using a variety of physiochemical techniques (e.g., lipofection, electroporation, etc.), or by transfection of DNA constructs encoding the compound (e.g., membrane-bound or transmembrane proteins) into the WNT-responsive reporter cell line and allowing the native machinery of the cells to produce the WNT modulator, and then exposing the treated cells to a WNT activator compound (e.g. WNT3A) and observing whether there is a measurable and significant change in luciferase activity compared to the control WNT-responsive reporter cells which do not express the WNT modulator. In one embodiment, the 293.TCF cells can be engineered to overexpress RNF43 or ZNRF3 proteins (e.g. 293.TCF.37 in the case of RNF43) and the luciferase activity in these lines compared to that in control 293.TCF cell lines that do not expresses RNF43 following exposure of both cell lines to appropriate WNT activators (e.g. conditioned medium). In such a way, the biological activity of RNF43 was first demonstrated (Koo et al, supra), the observations of which have been confirmed by the inventors in which it is demonstrated that RNF43 is a WNT modulator that decreases (or antagonizes) WNT signaling, as determined by observing a decrease in luciferase activity of the 293.TCF.37 WNT responsive reporter cell line that expresses RNF43 compared to the luciferase activity in the 293.TCF cell line which does not express RNF43 (See Example 8; FIG. 6B). The RNF43-mediated decrease or antagonism of WNT has been linked to the ability of RNF43, an E3-ubiquitin ligase to bind to and specifically tag FZD proteins for degradation, reducing WNT receptor density on the cell surface and reducing the response to an activating WNT signal (e.g. WNT3A ligand). WNT modulators may also be tested in more complex situations in which multiple modulators may be added to determine their additive effects upon WNT signaling. In one embodiment, the 293.TCF cells engineered to overexpress a known WNT modulator (e.g., 293.TCF.37 cells overexpressing RNF43) may be exposed to additional WNT modulators in order to determine whether such exposure results in a measurable and significant change in luciferase activity compared to control cells that were not exposed to the additional WNT modulator.

The R-spondin (RSPO) family of proteins, known to be involved in the WNT signaling pathway (Kim et al. 2008; PMID: 18400942; see FIG. 6B) has been confirmed by the inventors to be a WNT modulator that increases WNT signaling. This was demonstrated by observing an increase in luciferase activity of the 293.TCF.37 (e.g., RNF43-overexpressing) WNT responsive reporter cell line in the presence of WNT3A, and R-spondin (e.g. RSPO3), compared to the luciferase activity of the same cell line, 293.TCF.37, in the presence of WNT3A but in the absence of R-spondin (data not shown). Published molecular cell biological studies have shown both (1) that R-spondins are WNT modulators; they do not activate WNT signaling alone, e.g, in the absence of a WNT ligand, but instead positively modulate (i.e. increase) WNT signaling in the presence of a WNT ligand) and (2) that R-spondins' ability to increase WNT signaling is related to their ability to promote the interaction of LGR receptors with RNF43 or ZNRF3, resulting in the membrane clearance of RNF43 or ZNRF3, with subsequent promotion of increased FZD residence at the cell surface, thereby up-modulating or increasing WNT signaling.

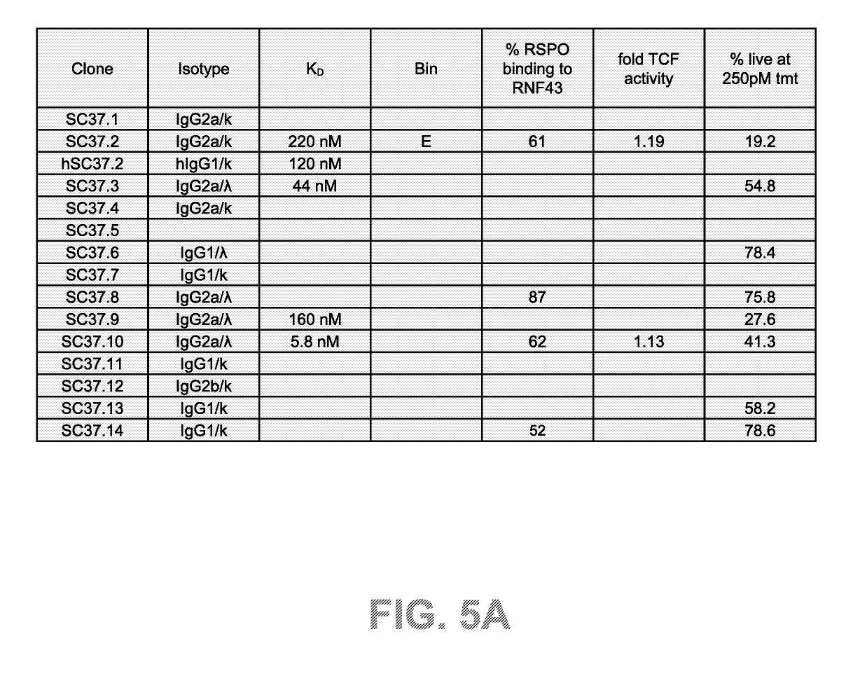

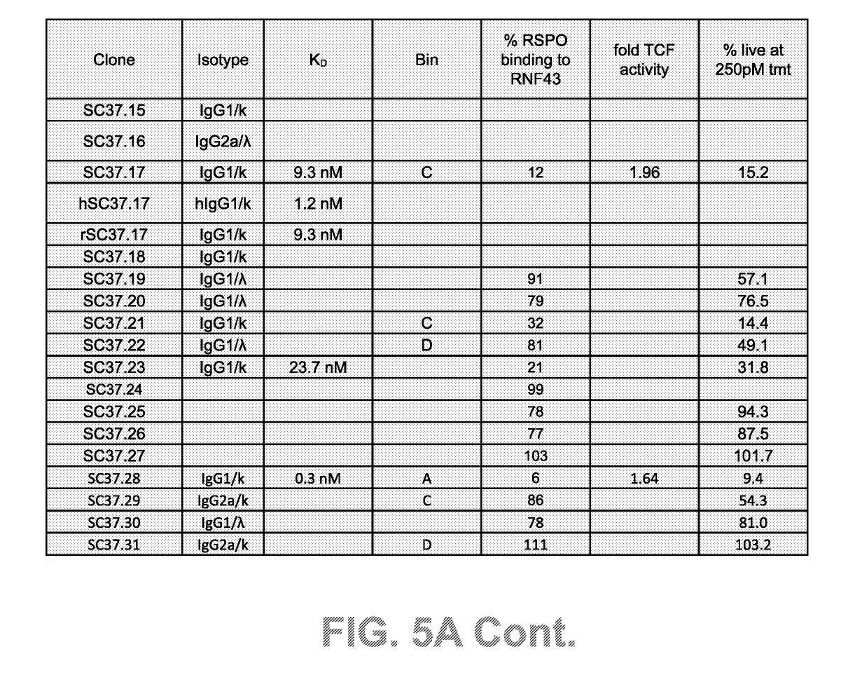

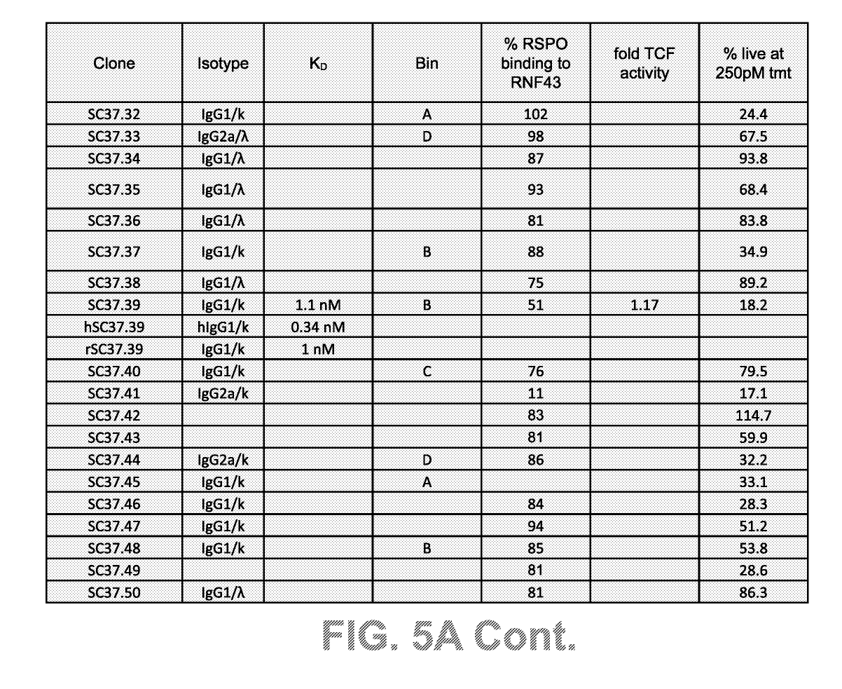

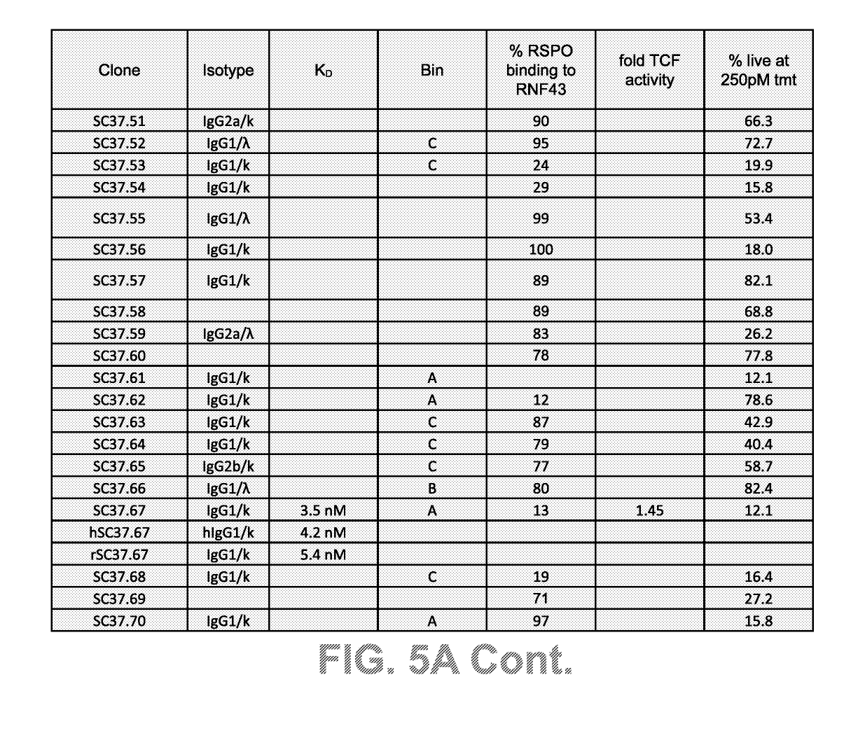

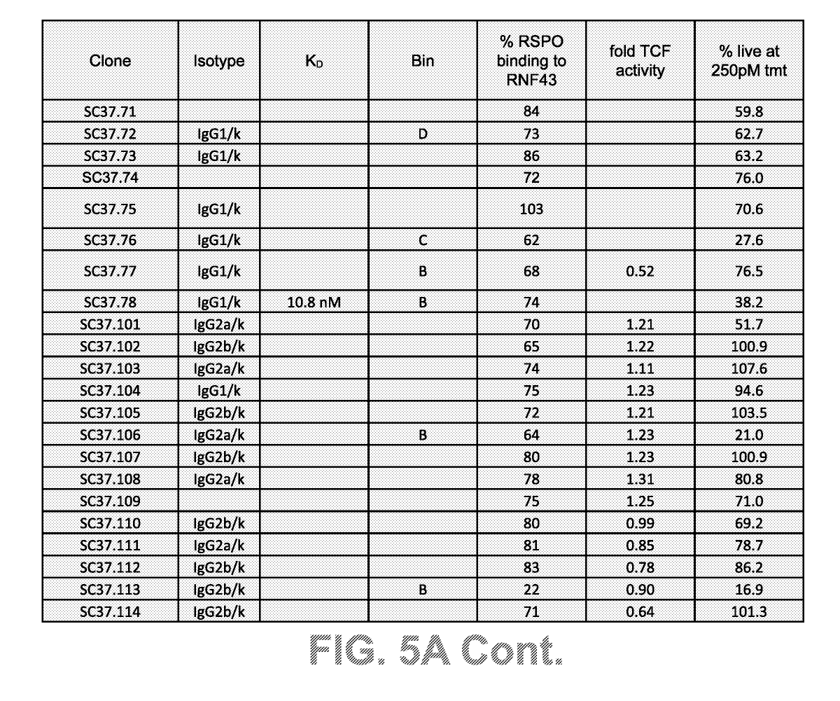

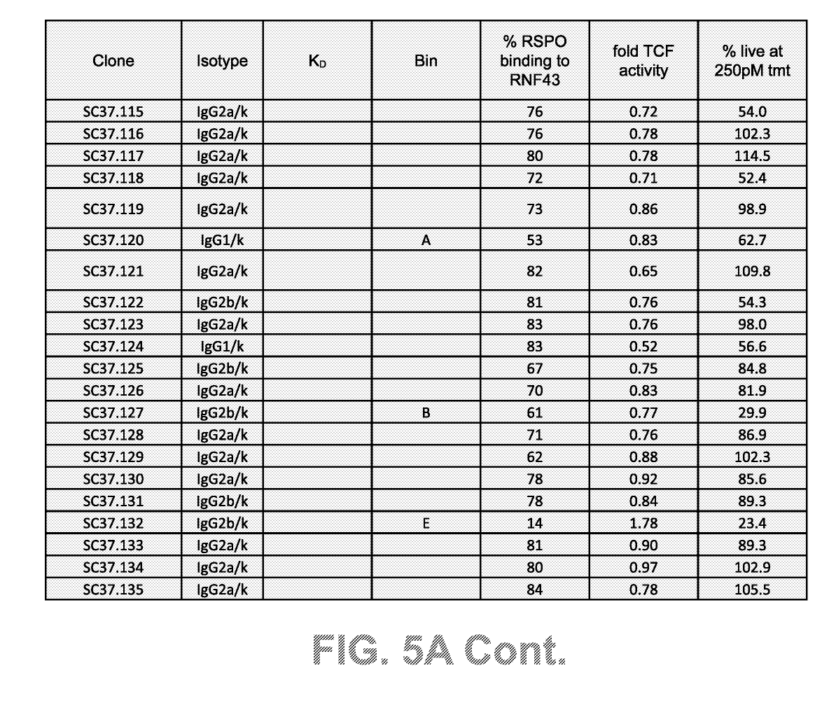

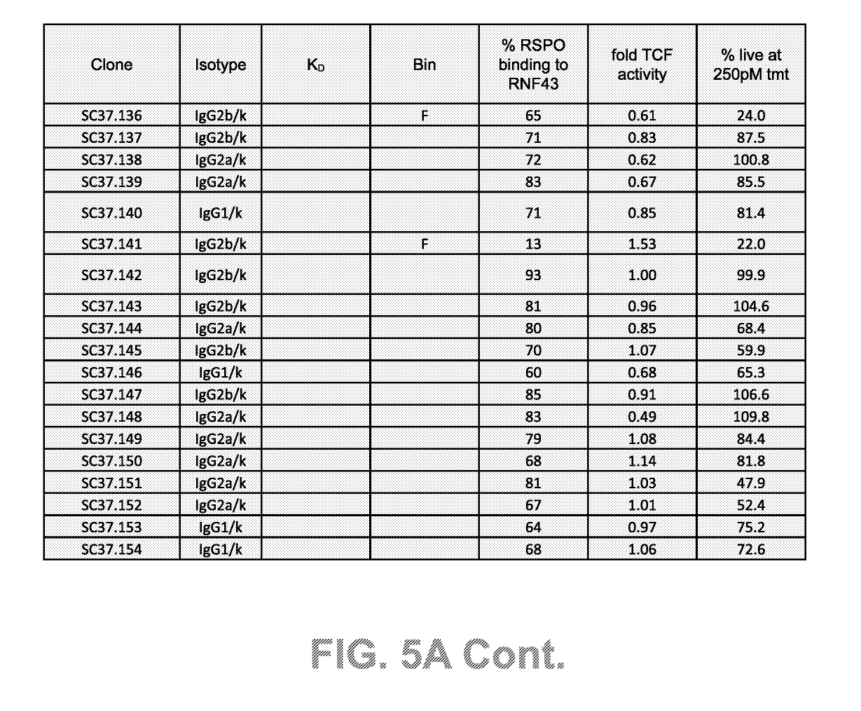

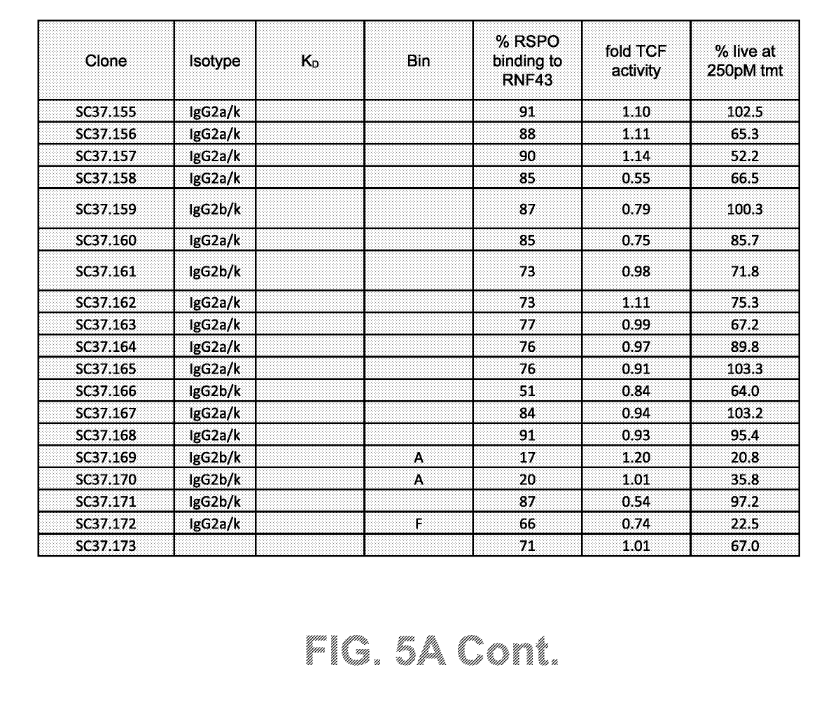

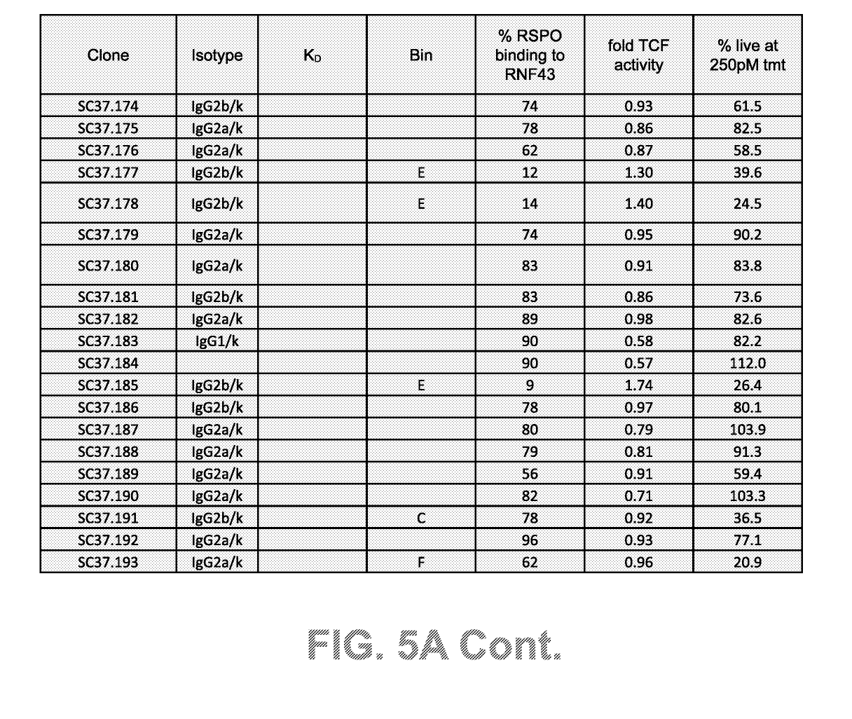

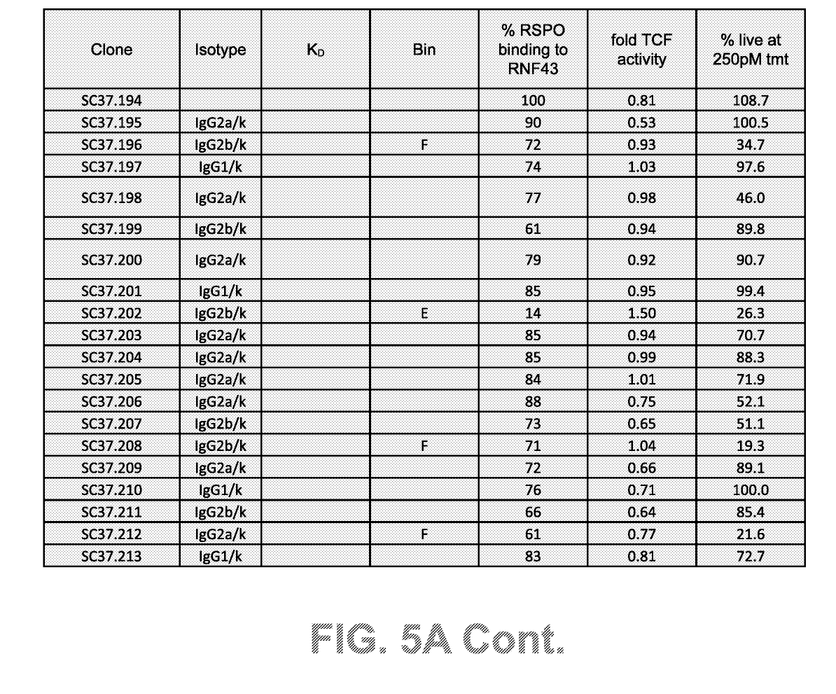

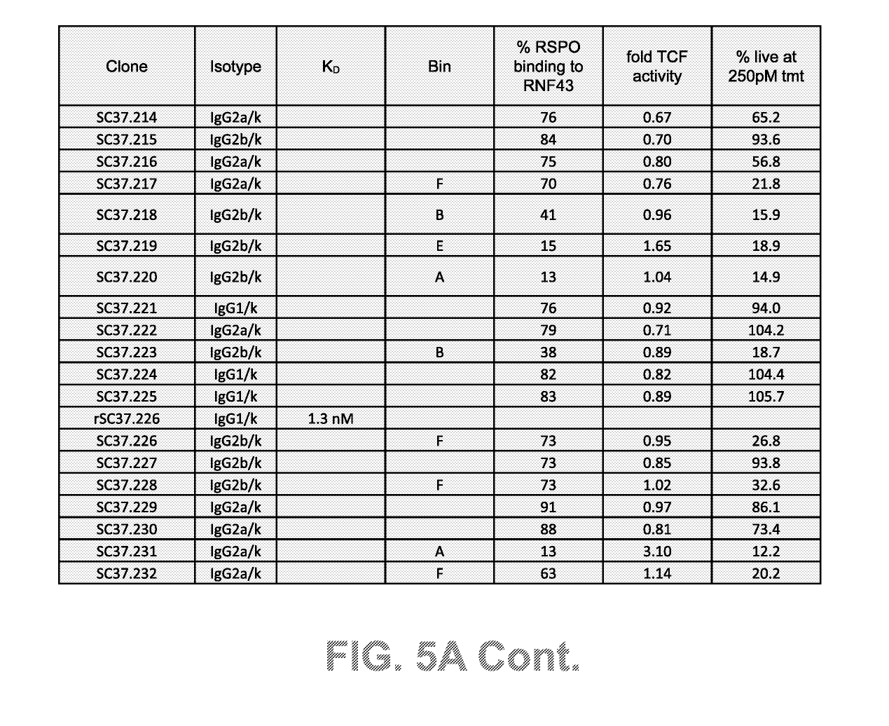

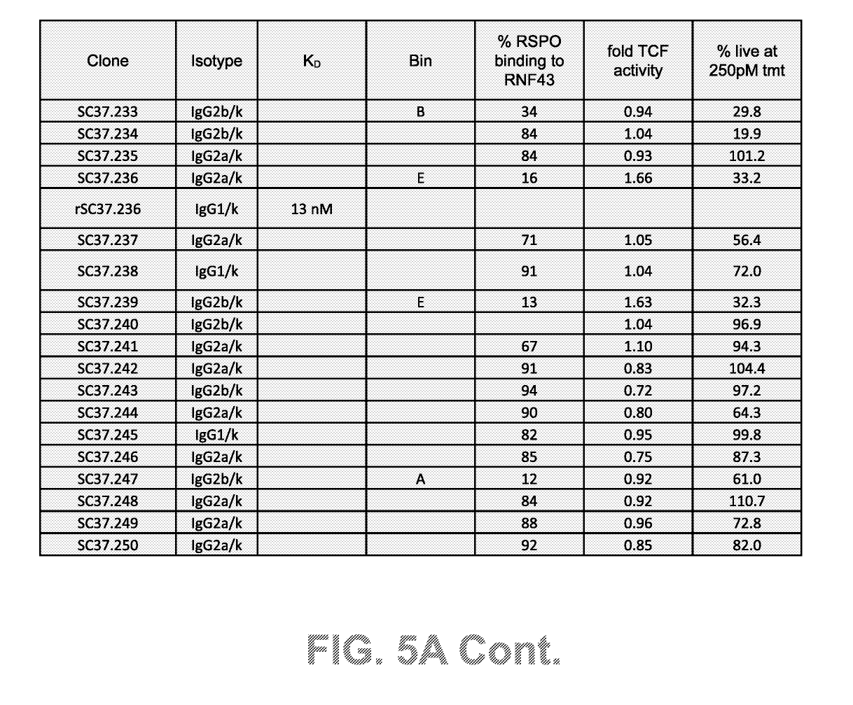

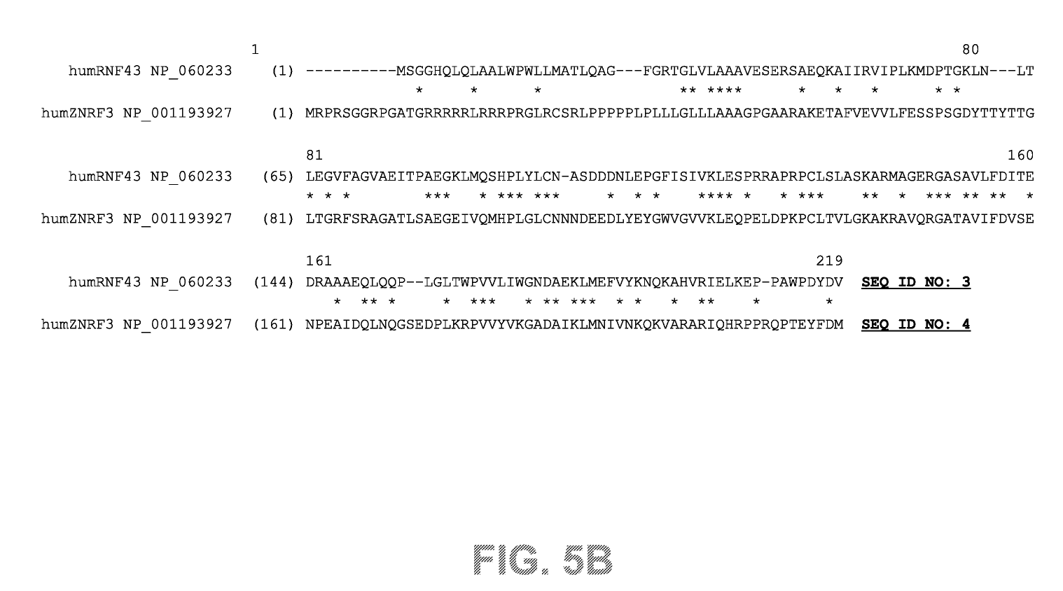

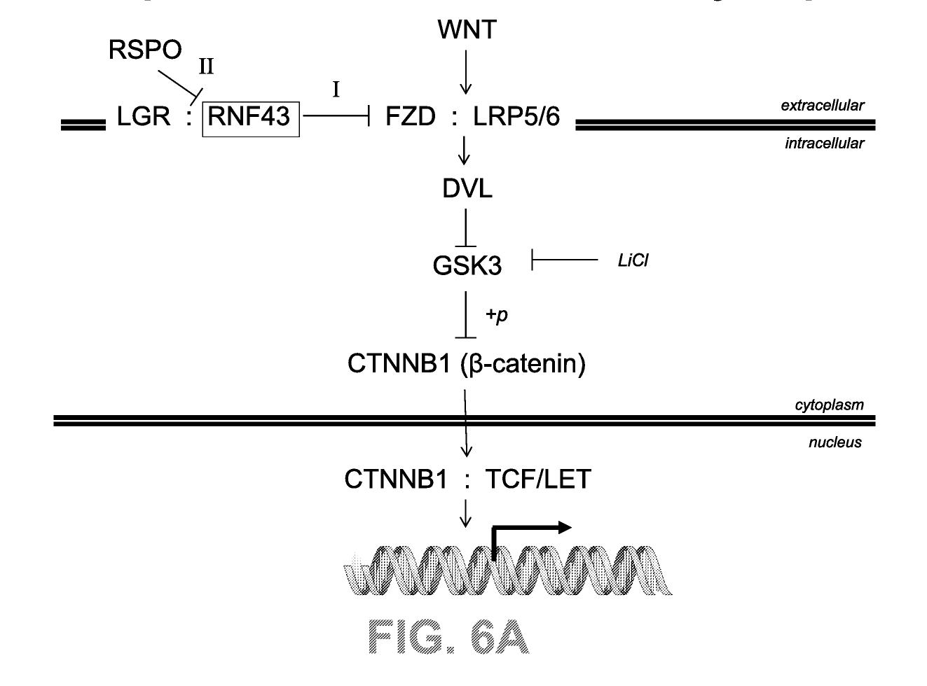

As used herein, relative terms such as “increases WNT signaling”, “decreases WNT signaling” or “does not affect WNT signaling” indicate the ability of various WNT modulators or WNT activators (e.g. the anti-RNF43 antibodies or antibody drug conjugates of the invention) alone or in combination to modulate the WNT signaling pathway relative to a control condition or to a reference compound. In one embodiment the ability of certain compounds (e.g. the anti-RNF43 antibodies or antibody drug conjugates of the invention) to modulate WNT signaling can be determined by exposing the 293.TCF WNT responsive reporter cell line to such compounds in the presence of a WNT ligand (e.g. WNT3A; see Example 8). Compounds that increase luciferase activity above that observed from exposure to WNT ligand alone are said to “increase WNT signaling” (e.g., the anti-RNF43 antibody SC37.231; see FIG. 5A); whereas compounds that do not change luciferase activity compared to that observed from exposure to WNT ligand alone are said to “not affect WNT signaling” (e.g., the anti-RNF43 antibody SC37.170; see FIG. 5A); and compounds that reduce luciferase activity below that observed from exposure to WNT ligand alone are said to “decrease WNT signaling” (e.g., the anti-RNF43 antibody SC37.231; see FIG. 5A). Changes in WNT signaling can be expressed as “fold TCF activity,” in which the TCF activity measured in the WNT reporter line in the test condition is divided by the TCF activity observed for the WNT reporter line in the control condition. An increase or a decrease in WNT signaling, compared to a control, is determined to be “significant” based on standard statistical techniques well known to the person of skill in the art (e.g., Student T-test; p value); hence measurable changes are considered statistically significant provided they are outside the range of normal biological variability for the experimental assay. For example, if the assay can reliably distinguish the statistically significant differences in test versus control populations where the mean of each population differs from the other population by a factor of 2 or more i.e., a 2-fold difference, a WNT modulator that “significantly increases WNT signaling” can be said to increase WNT signaling (e.g. TCF activity) by 2.0 fold or more (e.g. a ratio of the test and control population of 2.1, 2.2, 2.3, 2.4, 2.5, 0.2.6, 2.7, 2.8, 2.9, 3.0 or greater) Likewise, a WNT modulator that “significantly decreases WNT signaling” can be said to decrease WNT signaling (e.g. TCF activity) by 2 fold or more (e.g. a ratio of the test and control population of 0.5, 0.4, 0.3, 0.2, 0.1 or less). WNT modulators that have “no significant effect on WNT signaling” (e.g. neutral antibodies) can, for example, be said to change WNT signaling (either increase WNT signaling or decrease WNT signaling) by less than 2.0 fold.

3. Modulation of WNT Signaling by Anti-RNF43 Antibodies and Antibody Drug Conjugates

A simplified map of the canonical WNT signaling pathway is shown in FIG. 6A. The ability of the anti-RNF43 antibodies to modulate WNT signaling can be determined using various methods, some of which are described above in Section I.2. of the current specification. As described above, the activity of the antibodies as WNT modulators can be determined using WNT reporter assays, which provide direct readouts of WRE activated transcription and therefore directly measure WNT signaling. In the instant invention, the measurement of TCF activity in the presence of WNT3A ligand and anti-RNF43 antibodies in comparison to the TCF activity in the presence of WNT3A ligands without anti-RNF43 antibodies (FIG. 5A) is an example of a direct measurement of the effect of a WNT modulator upon WNT signaling.

Additionally, other art-recognized assays can be used to infer WNT modulation based upon a known understanding of the biology of the proteins in the WNT signaling pathway. For example, the expression levels of the WNT receptor, FZD5, can be measured at the cell surface, since it is known that changes in the amount of the FZD5 receptor present at the cell surface correlate directly with changes in WNT signaling (see Koo et al., supra).

RNF43 is known to be a WNT modulator that decreases WNT signaling via a mechanism that involves physical interactions between RNF43 and FZD receptors, resulting in decreased levels of FZD on the cell surface and leading to decreased WNT signaling (Koo et al., supra; Hao et al., supra). Antibodies that functionally block the interaction of RNF43 with FZD (labeled as Group I antibodies in FIG. 6A) would be expected to cause an increase in FZD receptor density at the cell surface, resulting in increased WNT signaling. In one embodiment the ability of the anti-RNF43 antibodies or ADCs of the invention to increase WNT signaling can be indirectly determined by the ability of such anti-RNF43 antibodies and ADCs to compete with RNF43 for binding to FZD thereby preventing degradation of FZD, leading to increased WNT signaling. Such competition experiments can be conducted using an ELISA assay or other competition assays as described in more detail in Section IV.5 of the instant specification, wherein the ability of the anti-RNF43 antibodies to block the binding of RNF43 to an isolated FZD protein, a FZD ectodomain, or cells overexpressing FZD, is determined.

Similarly, it is known by those skilled in the art that R-spondin (RSPO) functionally blocks the activity of RNF43. Surface expression of RNF43 is important for RNF43's ability to modulate FZD receptors; and RSPO blocks the activity of RNF43 by recruiting the LGR4 receptor into a ternary complex consisting of RSPO, RNF43, and LGR4, thereby sequestering RNF43 from FZD, resulting in increased levels of FZD (a WNT receptor) on the cell surface that leads to increased WNT signaling (Hao et al., supra; Zebisch et al., 2013, PMID 24225776; Xie et al. 2013, PMID: 24165923). Therefore antibodies which functionally block the interaction of RSPO with RNF43 (labeled as Group II antibodies in FIG. 6A) would be expected to cause a decrease in FZD receptor density at the cell surface, and result in decreased WNT signaling relative to control conditions with RSPO in the absence of antibody. In one embodiment WNT modulator activity by anti-RNF43 antibodies and ADCs can be indirectly determined by the ability of such anti-RNF43 antibodies and ADCs to compete with R-spondin for binding to RNF43 and therefore block binding of R-spondin to RNF43. Such competition experiments can be conducted using an ELISA assay, for example, essentially as follows: anti-RNF43 antibodies can be added to recombinant RNF43 extracellular domain protein, and the mixture added to an ELISA plate coated with R-spondin (See Example 8; FIG. 5A) to determine whether the antibodies block the interaction of R-spondin with RNF43. An anti-RNF43 antibody or ADC that blocks R-spondin interaction with RNF43 is held to mean an antibody which shows a reduction in the binding of R-spondin to RNF43 by, for example, 50%, 60%, 70%, 80%, 90% or above, compared to the binding of R-spondin to RNF43 in the absence of the antibody. In other embodiments, additional competition assays can be performed as described in more detail in Section IV.5 of the instant specification. The indirect measurements of WNT signaling as measured by the activity of WNT modulators (e.g. R-spondins and FZD) can be confirmed using the direct WNT signaling assays described in Section I.2. of the current specification.

Finally, it can be anticipated that there will exist anti-RNF43 antibodies and ADCs, which show neither of the properties of Group I or Group II antibodies, described above i.e. such antibodies or ADCs will not block R-spondin interaction with RNF43, nor will they block RNF43 interaction with FZD. This group of antibodies or ADCs, while still able to bind RNF43 specifically, can be termed “neutral antibodies”, due to the fact that they will have no effect on WNT signaling.

### II. CANCER STEM CELLS

According to the current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which make up 0.1-40% of a tumor's cell population, have the ability to form tumors. Tumorigenic cells encompass both tumor perpetuating cells (TPCs), referred to interchangeably as cancer stem cells (CSCs) and tumor progenitor cells (TProgs).

CSCs, like normal stem cells that support cellular hierarchies in normal tissue, are able to self-replicate indefinitely while maintaining the capacity for multilineage differentiation. CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice.

TProgs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because TProgs are typically only capable of a finite number of cell divisions as demonstrated by serial transplantation of low numbers of highly purified TProg into immunocompromised mice. TProgs may further be divided into early TProgs and late TProgs, which may be distinguished by phenotype (e.g., cell surface markers) and their different capacities to recapitulate tumor cell architecture. While neither can recapitulate a tumor to the same extent as CSCs, early TProgs have a greater capacity to recapitulate the parental tumor's characteristics than late TProgs. Notwithstanding the foregoing distinctions, it has been shown that some TProg populations can, on rare occasion, gain self-renewal capabilities normally attributed to CSCs and can themselves become CSCs.

CSCs exhibit higher tumorigenicity and are relatively more quiescent than: (i) TProgs (both early and late TProgs); and (ii) non-tumorigenic cells such as tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are more resistant to conventional therapies and regimens than the faster proliferating TProgs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. These properties in CSCs constitute a key reason for the failure of standard oncology treatment regimens to ensure long-term benefit for most patients with advanced stage neoplasia because standard chemotherapy does not target the CSCs that actually fuel continued tumor growth and recurrence.

It has surprisingly been discovered that RNF43 expression is associated with various tumorigenic cell subpopulations. The invention provides anti-RNF43 antibodies that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, or otherwise inhibit (collectively, “inhibit”) tumorigenic cells, thereby facilitating the treatment, management and/or prevention of proliferative disorders (e.g. cancer). Advantageously, the novel anti-RNF43 antibodies of the invention may be selected so they preferably reduce the frequency or tumorigenicity of tumorigenic cells upon administration to a subject regardless of the form of the RNF43 determinant (e.g., phenotypic or genotypic). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition of the tumorigenic cells, modification of their potential (for example, by induced differentiation or niche disruption) or otherwise interfering with the ability of tumorigenic cells to influence the tumor environment or other cells, allows for the more effective treatment of RNF43 associated disorders by inhibiting tumorigenesis, tumor maintenance and/or metastasis and recurrence.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

In vitro limiting dilution analysis may be performed by culturing fractionated or unfractionated tumor cells (e.g. from treated and untreated tumors, respectively) on solid medium that fosters colony formation and counting and characterizing the colonies that grow. Alternatively, the tumor cells can be serially diluted onto plates with wells containing liquid medium and each well can be scored as either positive or negative for colony formation at any time after inoculation but preferably more than 10 days after inoculation.

In vivo limiting dilution is performed by transplanting tumor cells, from either untreated controls or from tumors exposed to selected therapeutic agents, into immunocompromised mice in serial dilutions and subsequently scoring each mouse as either positive or negative for tumor formation. The scoring may occur at any time after the implanted tumors are detectable but is preferably done 60 or more days after the transplant. The analysis of the results of limiting dilution experiments to determine the frequency of tumorigenic cells is preferably done using Poisson distribution statistics or assessing the frequency of predefined definitive events such as the ability to generate tumors in vivo or not (Fazekas et al., 1982, PMID: 7040548).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

The antibodies of the invention may be useful for identifying, characterizing, monitoring, isolating, sectioning or enriching populations or subpopulations of tumorigenic cells through methods such as, for example, flow cytometry, magnetic activated cell sorting (MACS), laser mediated sectioning or FACS. FACS is a reliable method used to isolate cell subpopulations at more than 99.5% purity based on specific cell surface markers. Other compatible techniques for the characterization and manipulation of tumorigenic cells including CSCs can be seen, for example, in U.S. patent Ser. Nos. 12/686,359, 12/669,136 and 12/757,649.

Listed below are markers that have been associated with CSC populations and have been used to isolate or characterize CSCs: ABCA1, ABCA3, ABCG2, ADAM9, ADCY9, ADORA2A, AFP, AXIN1, B7H3, BCL9, Bmi-1, BMP-4, C20orf52, C4.4A, carboxypeptidase M, CAV1, CAV2, CD105, CD133, CD14, CD16, CD166, CD16a, CD16b, CD2, CD20, CD24, CD29, CD3, CD31, CD324, CD325, CD34, CD38, CD44, CD45, CD46, CD49b, CD49f, CD56, CD64, CD74, CD9, CD90, CEACAM6, CELSR1, CPD, CRIM1, CX3CL1, CXCR4, DAF, decorin, easyh1, easyh2, EDG3, eed, EGFR, ENPP1, EPCAM, EPHA1, EPHA2, F1110052, FLVCR, FZD1, FZD10, FZD2, FZD3, FZD4, FZD6, FZD7, FZD8, FZD9, GD2, GJA1, GLI1, GLI2, GPNMB, GPR54, GPRC5B, IL1R1, IL1RAP, JAM3, Lgr5, Lgr6, LRP3, LY6E, MCP, mf2, mllt3, MPZL1, MUC1, MUC16, MYC, N33, Nanog, NB84, nestin, NID2, NMA, NPC1, oncostatin M, OCT4, OPN3, PCDH7, PCDHA10, PCDHB2, PPAP2C, PTPN3, PTS, RARRES1, SEMA4B, SLC19A2, SLC1A1, SLC39A1, SLC4A11, SLC6A14, SLC7A8, smarcA3, smarcD3, smarcEl, smarcA5, Sox1, STAT3, STEAP, TCF4, TEM8, TGFBR3, TMEPAI, TMPRSS4, transferrin receptor, TrkA, WNT10B, WNT16, WNT2, WNT2B, WNT3, WNT5A, YY1 and β-catenin. See, for example, Schulenburg et al., 2010, PMID: 20185329, U.S. Pat. No. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes.

“Positive,” “low” and “negative” expression levels as they apply to markers or marker phenotypes are defined as follows. Cells with negative expression (i.e. “−”) are herein defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95th percentile of expression observed with an isotype control antibody using the FMO staining procedure described above are herein defined as “positive” (i.e. “+”). As defined herein there are various populations of cells broadly defined as “positive.” A cell is defined as positive if the mean observed expression of the antigen is above the 95th percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99th percentile may preferably be used as a demarcation point between negative and positive FMO staining and in particularly preferred embodiments the percentile may be greater than 99%.

The CD46^{hi}CD324⁺marker phenotype and those exemplified immediately above may be used in conjunction with standard flow cytometric analysis and cell sorting techniques to characterize, isolate, purify or enrich TIC and/or TPC cells or cell populations for further analysis.

The ability of the antibodies of the current invention to reduce the frequency of tumorigenic cells can therefore be determined using the techniques and markers described above. In some instances, the anti-RNF43 antibodies may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed compounds my reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. ANTIBODIES

1. Antibody Structure

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

As used herein, an “intact antibody” typically refers to a Y-shaped tetrameric protein comprising two heavy (H) and two light (L) polypeptide chains held together by covalent disulfide bonds and non-covalent interactions. Human light chains are classified as kappa or lambda light chains. Each light chain is composed of one variable domain (VL) and one constant domain (C_{L}). Each heavy chain comprises one variable domain (VH) and a constant region, which in the case of IgG, IgA, and IgD, comprises three domains termed C_{H}1, C_{H}2, and C_{H}3 (IgM and IgE have a fourth domain, C_{H}4). In IgG, IgA, and IgD classes the C_{H}1 and C_{H}2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. ScFv fragments (for single chain fragment variable), which can be obtained by genetic engineering, associates in a single polypeptide chain, the VH and the VL region of an antibody, separated by a peptide linker.

As used herein, the assignment of amino acids to each domain, framework region and CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. The amino acid residues which comprise CDRs as defined by Kabat, Chothia, MacCallum (also known as Contact) and AbM as obtained from the Abysis website database (infra.) are set out below.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

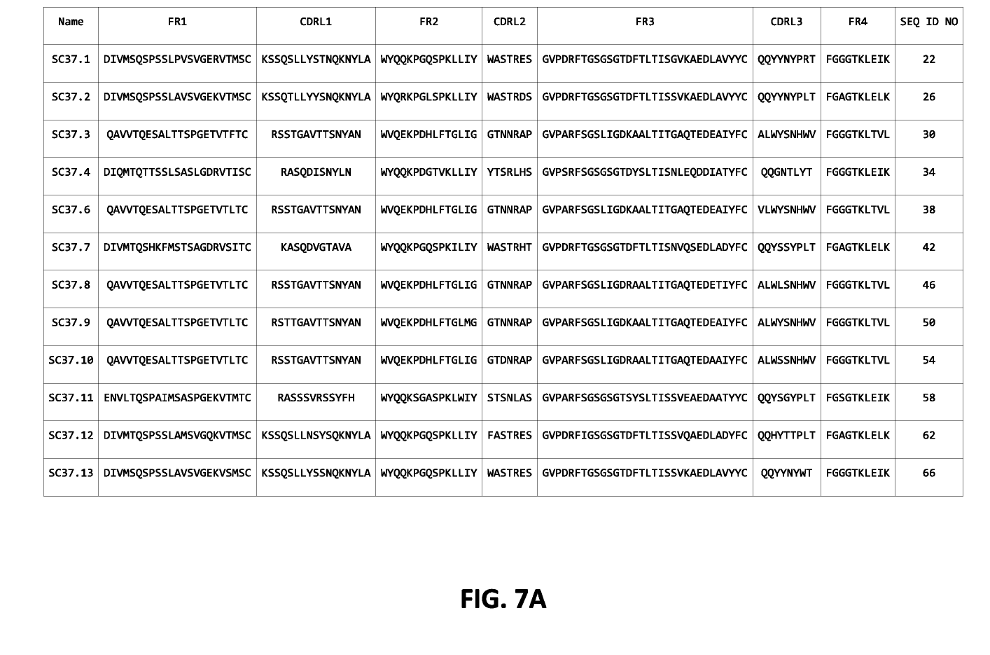

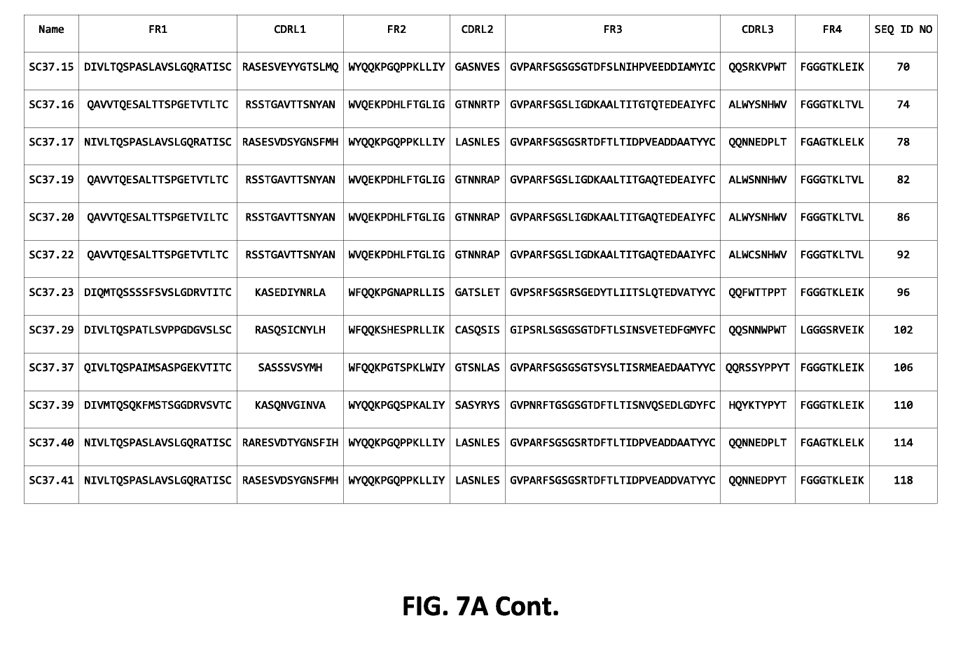

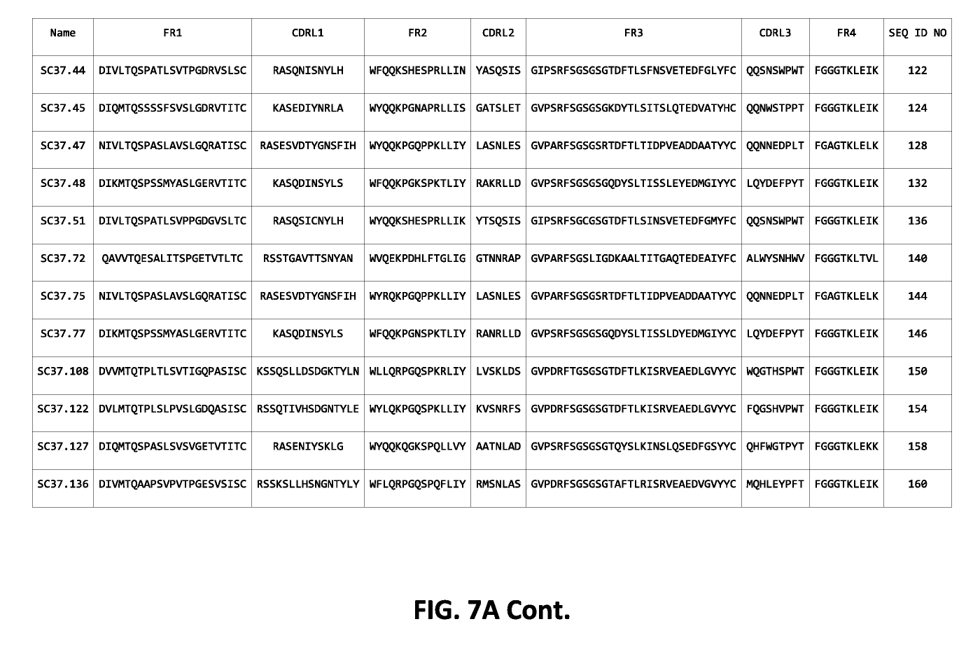

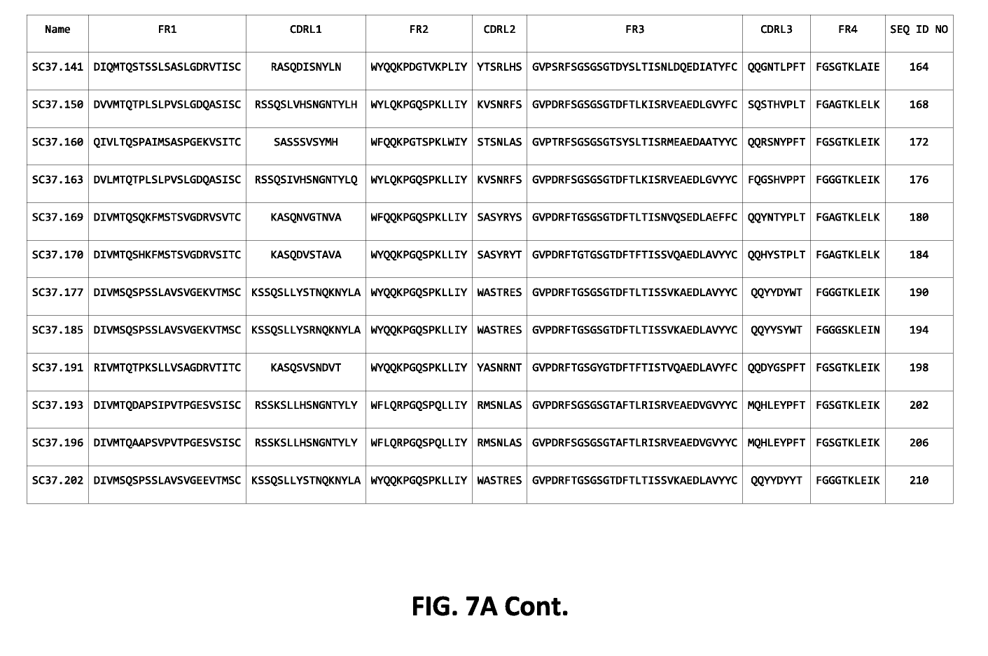

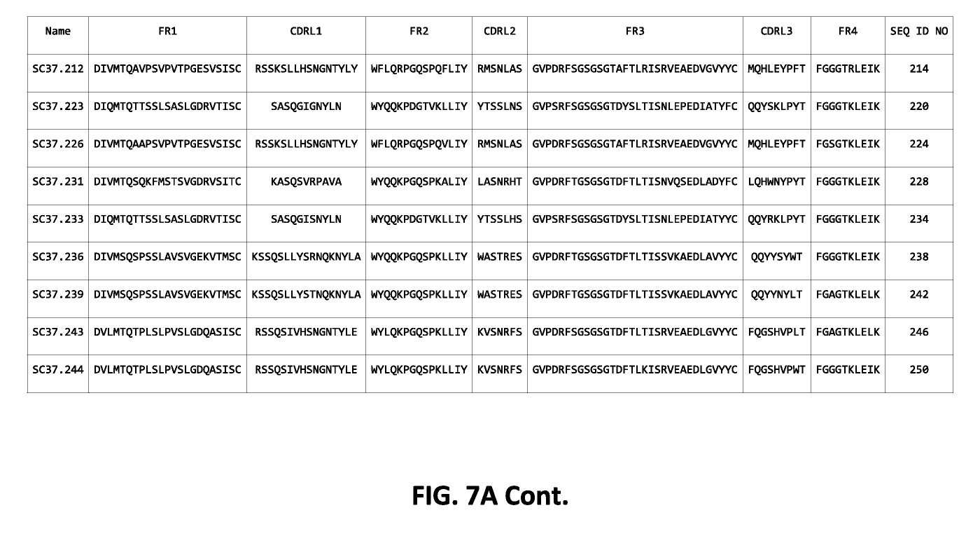

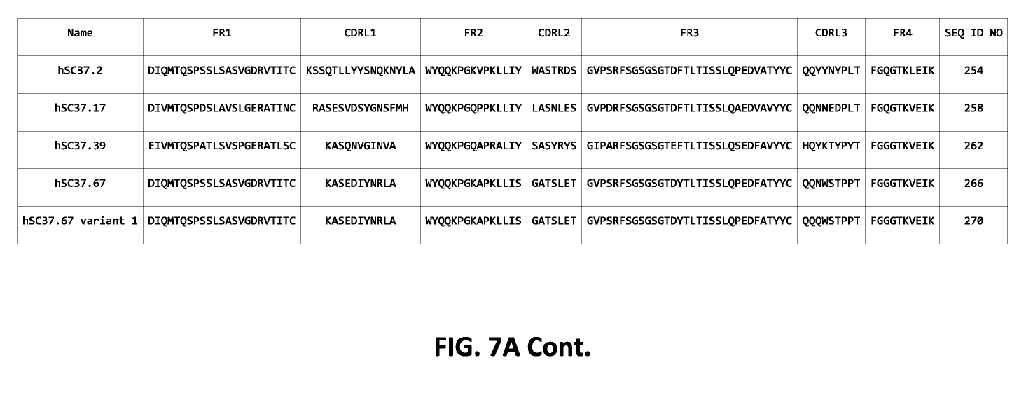

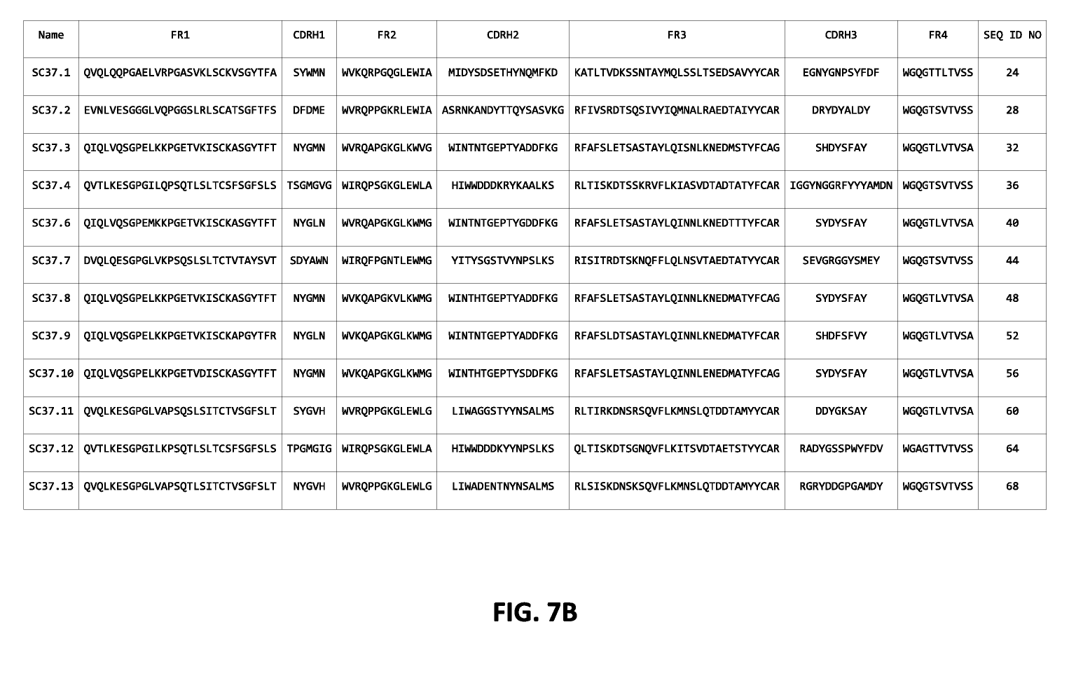

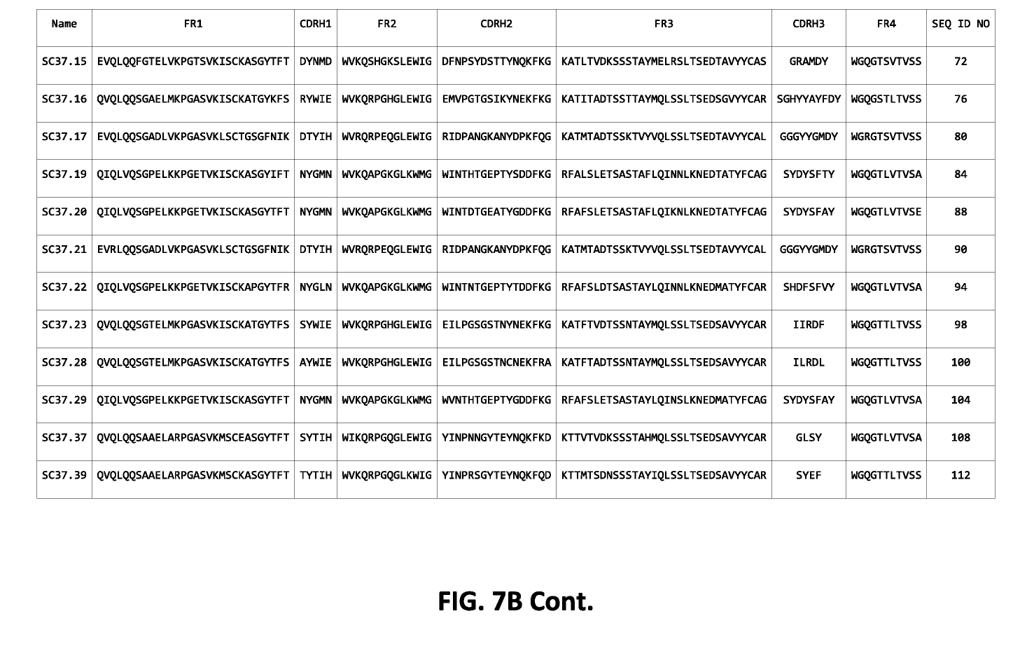

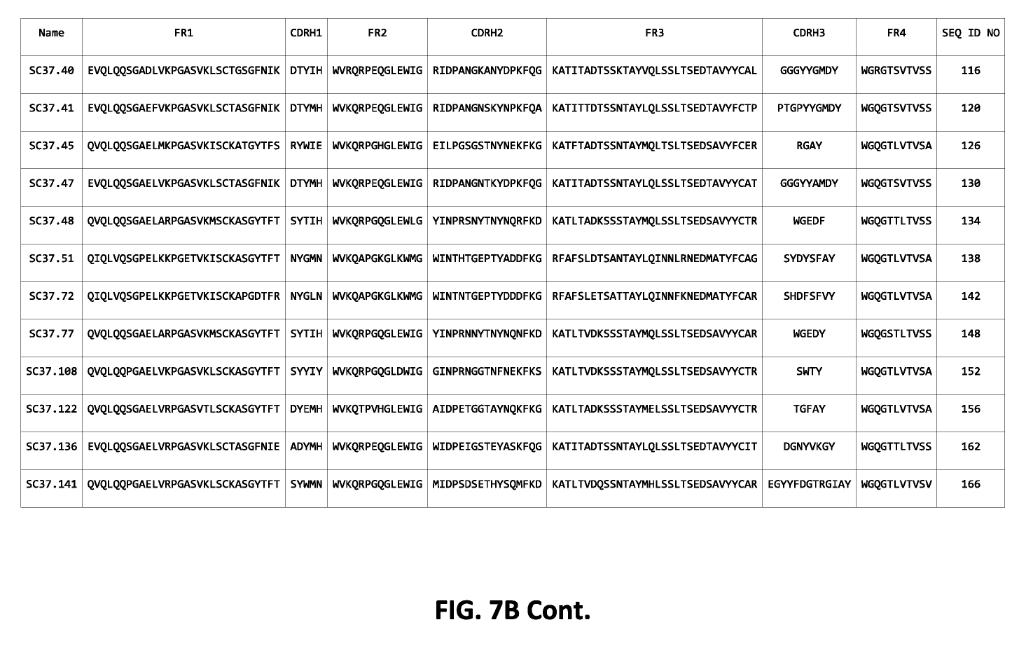

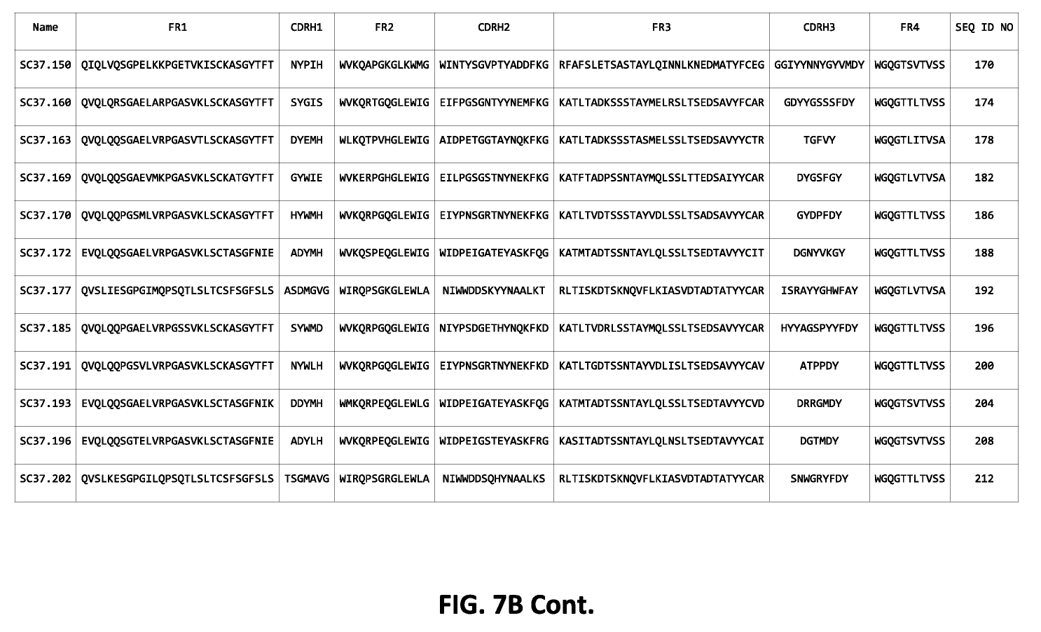

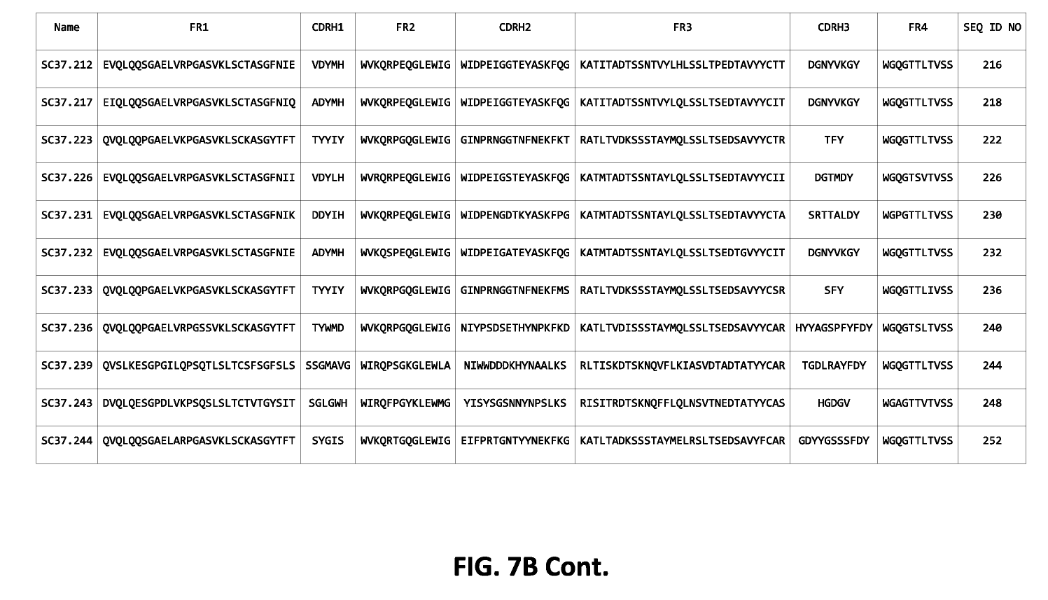

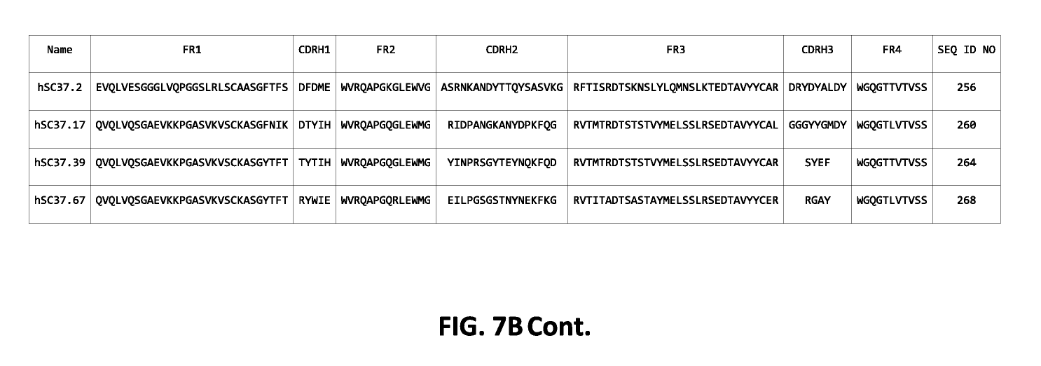

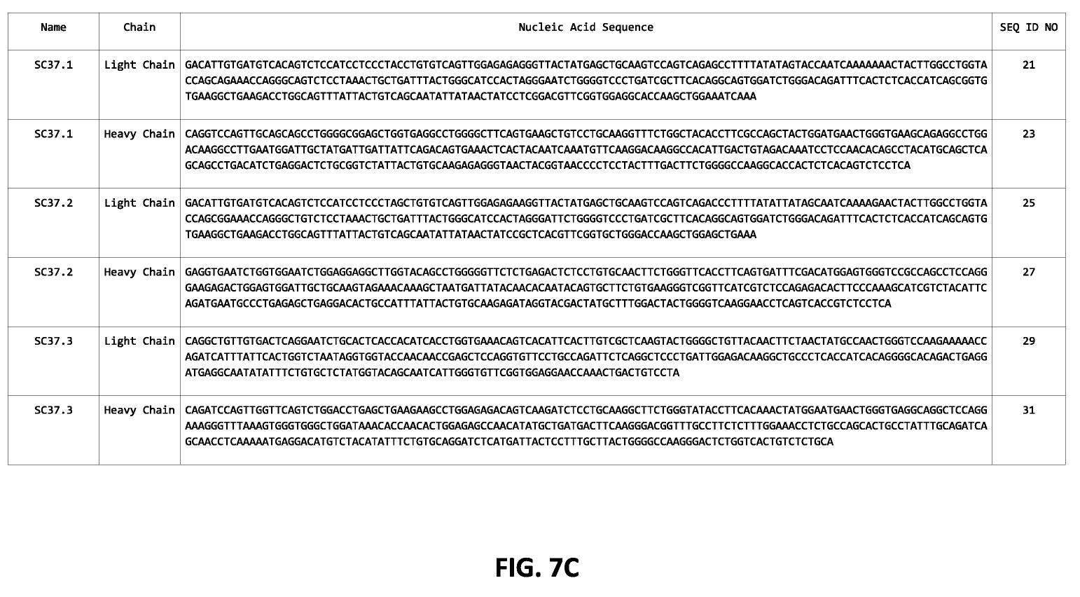

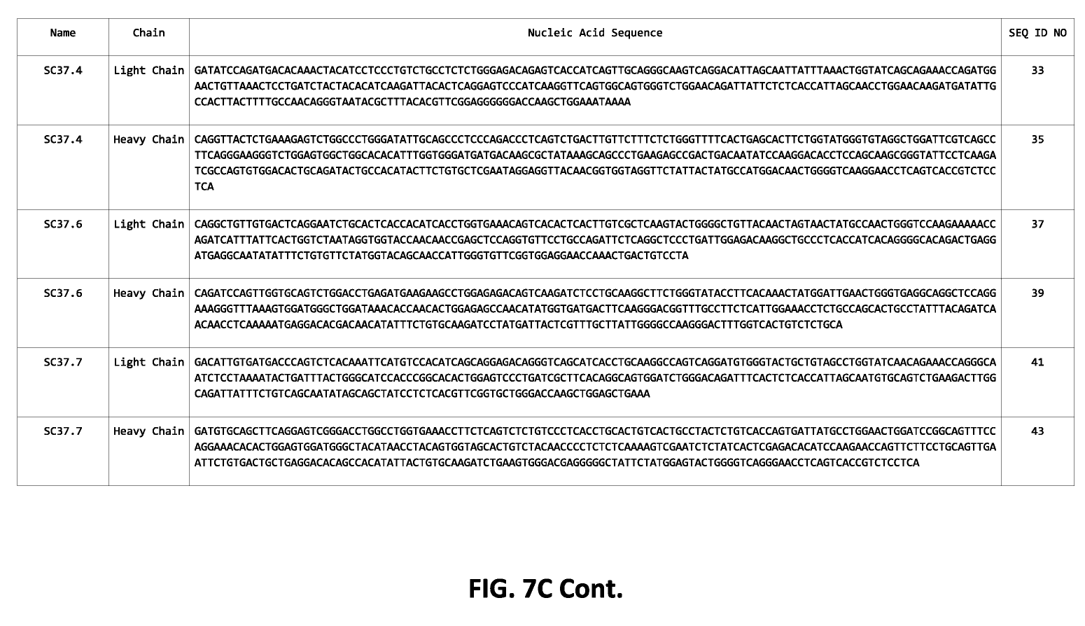

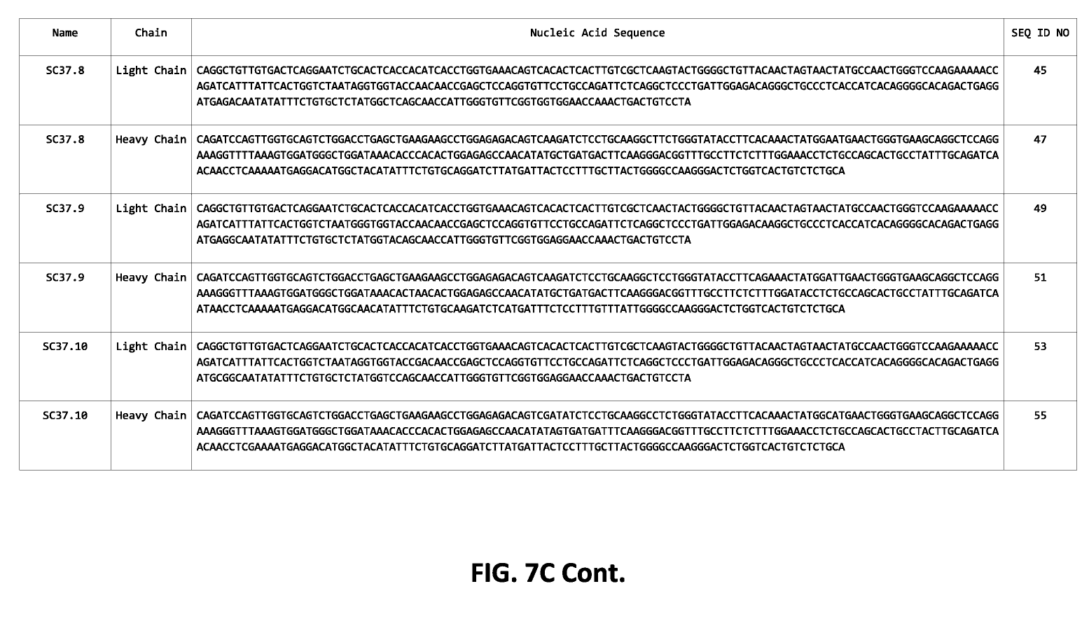

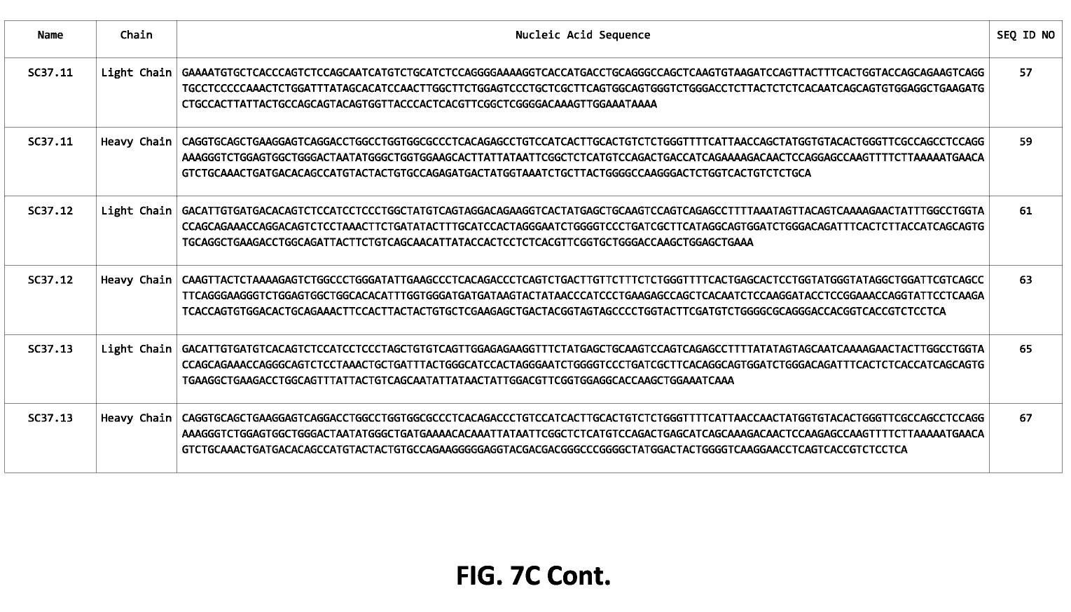

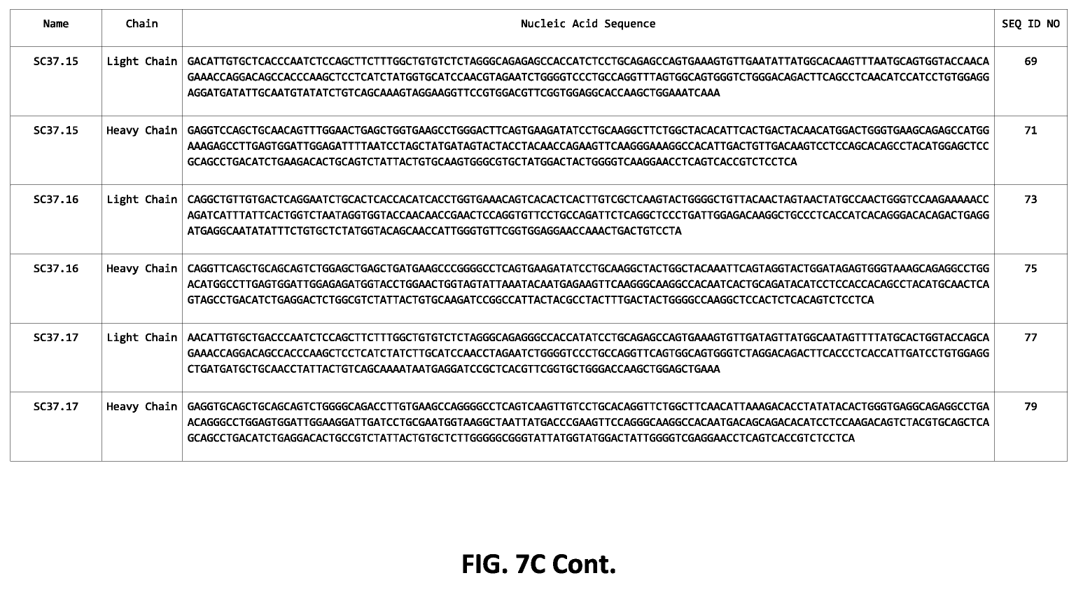

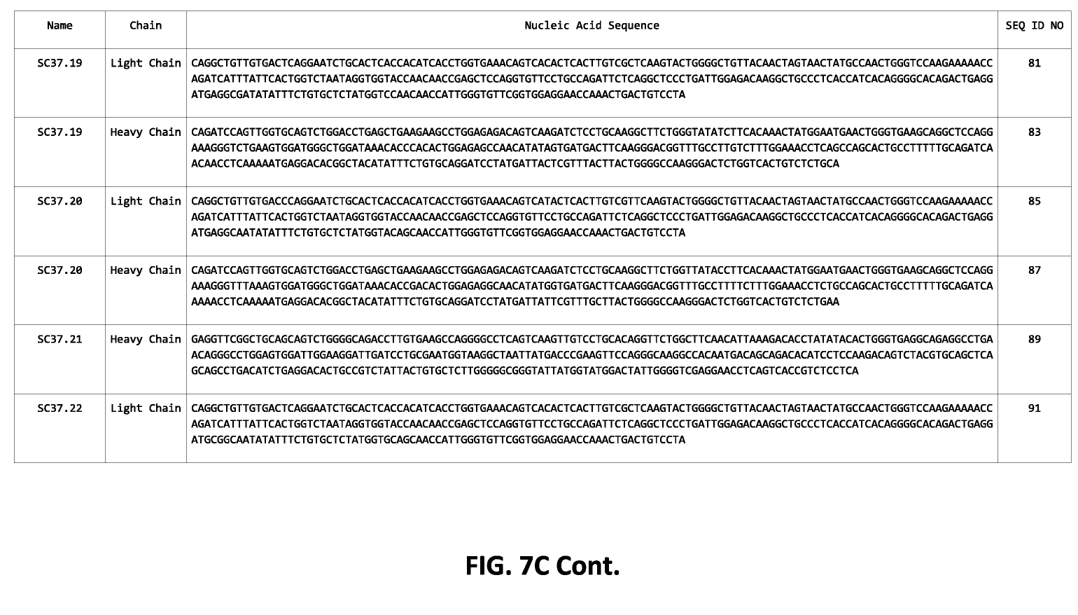

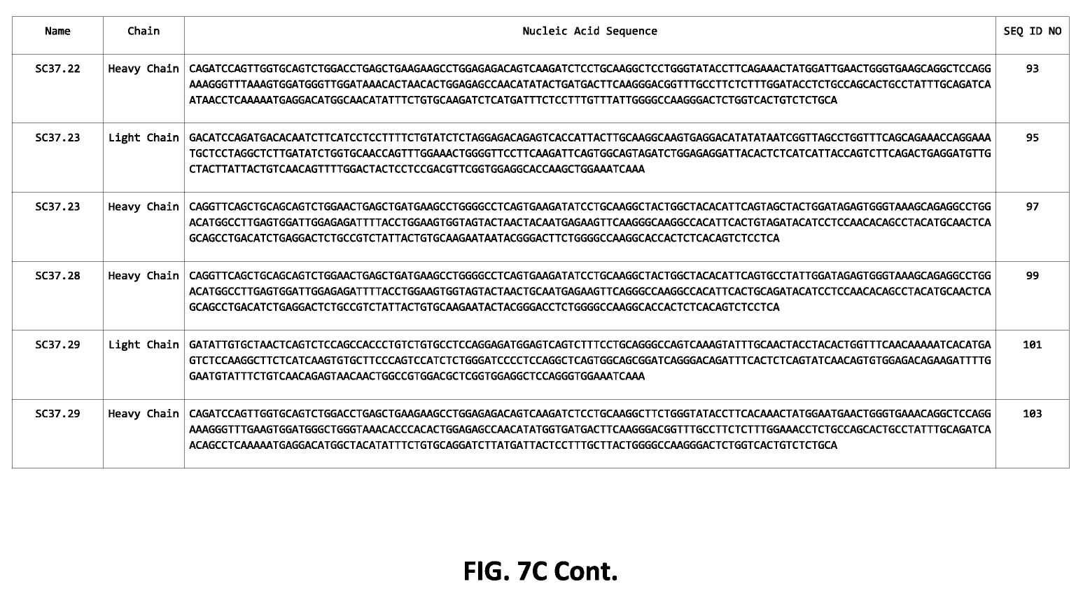

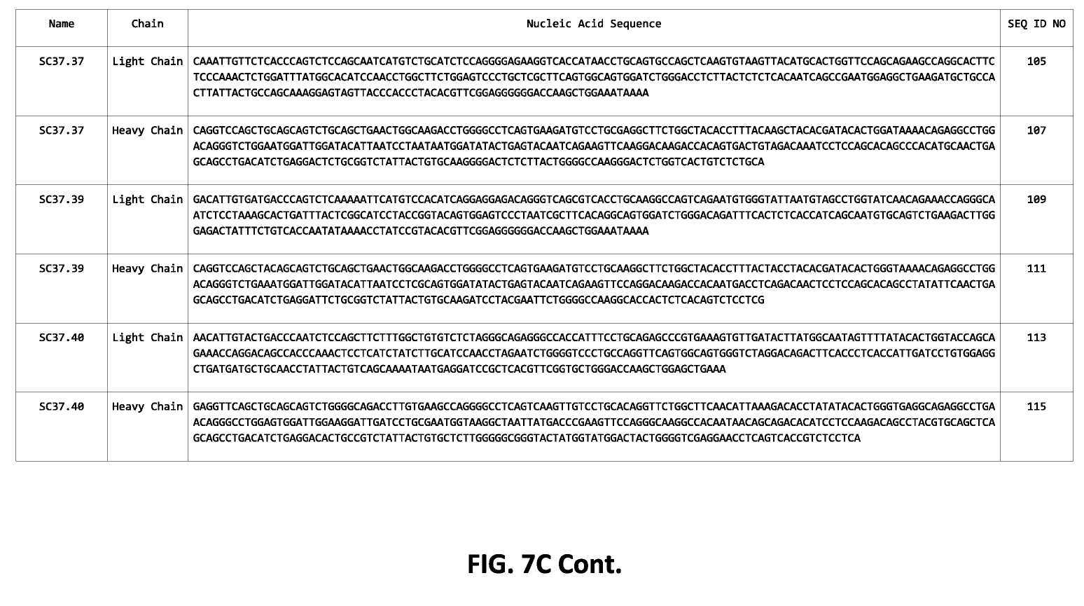

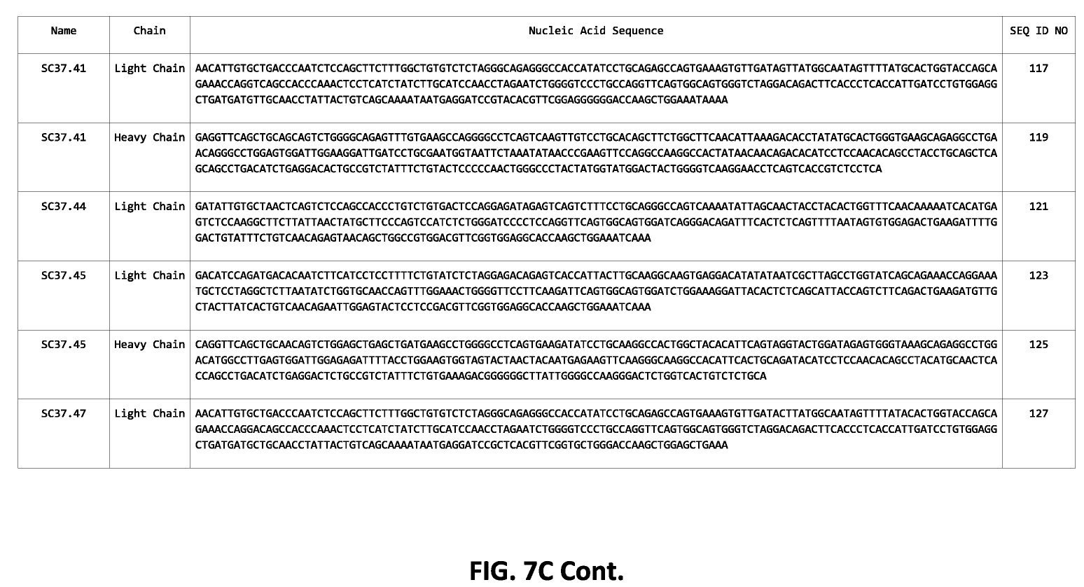

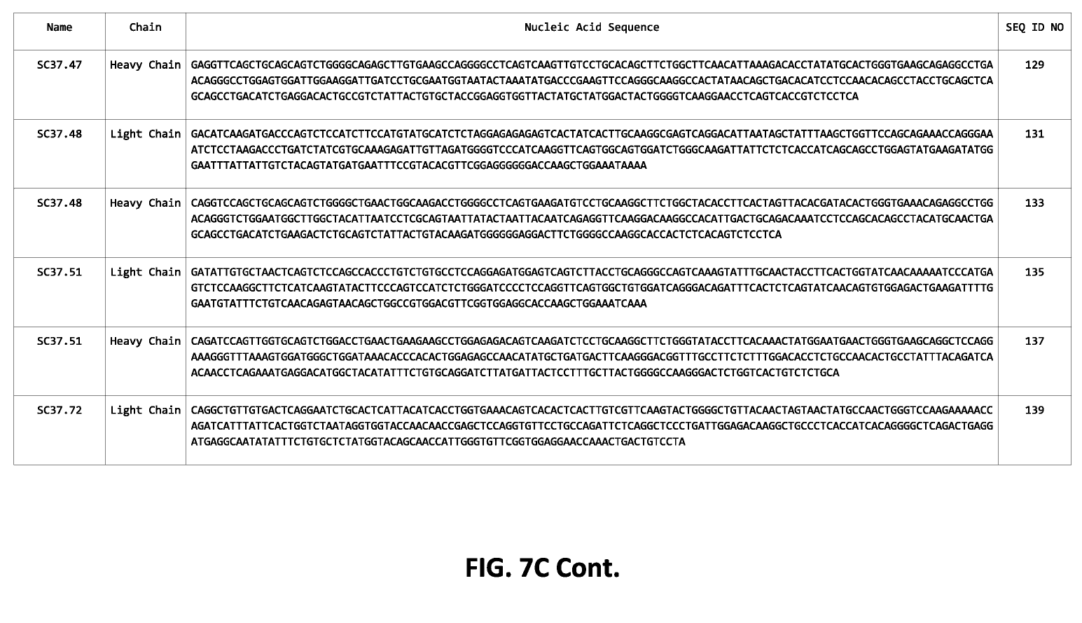

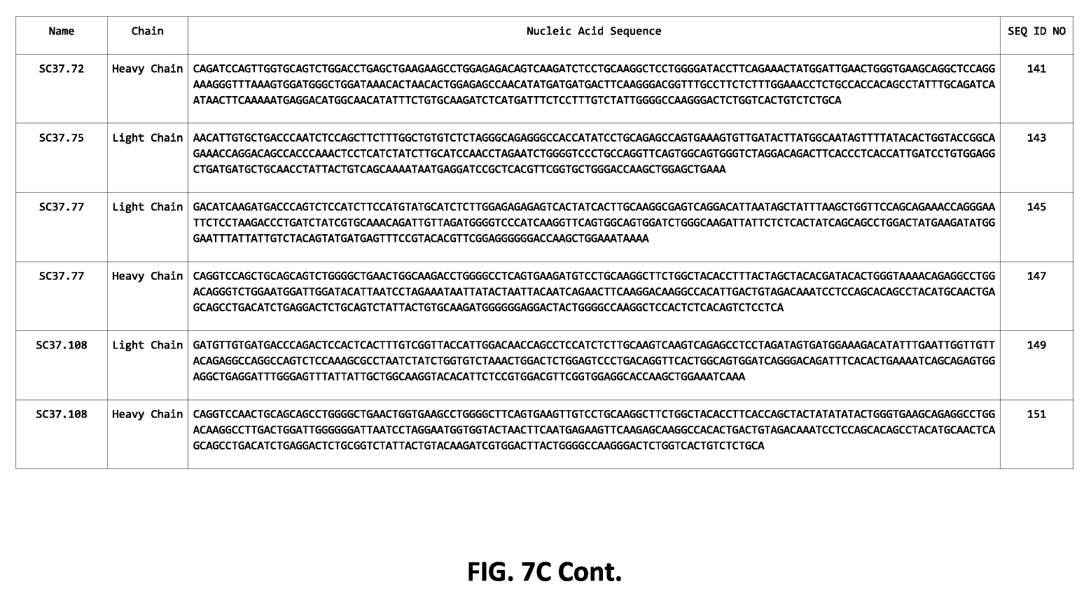

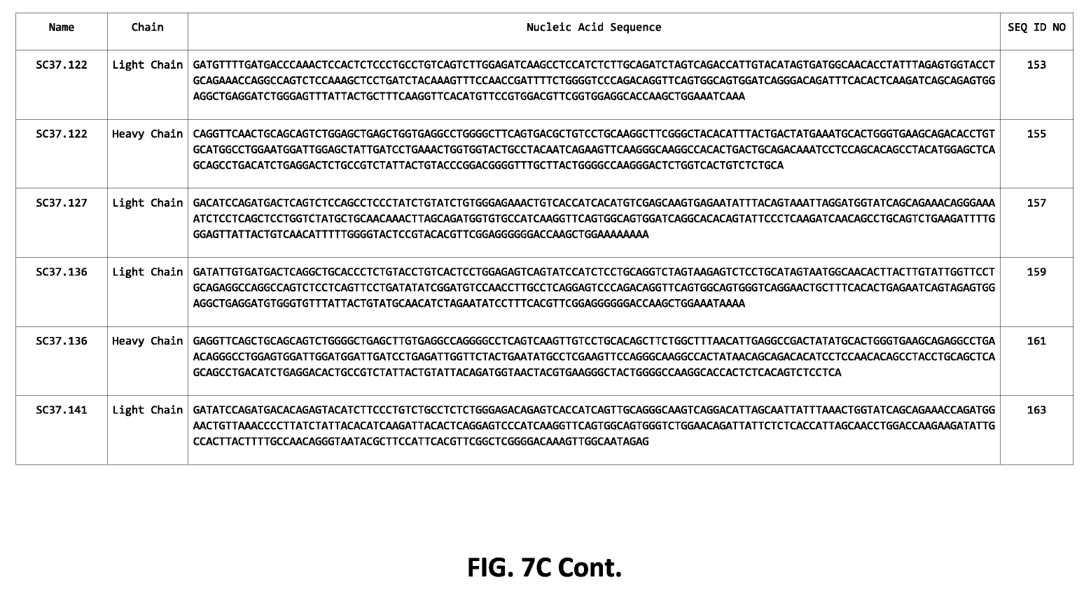

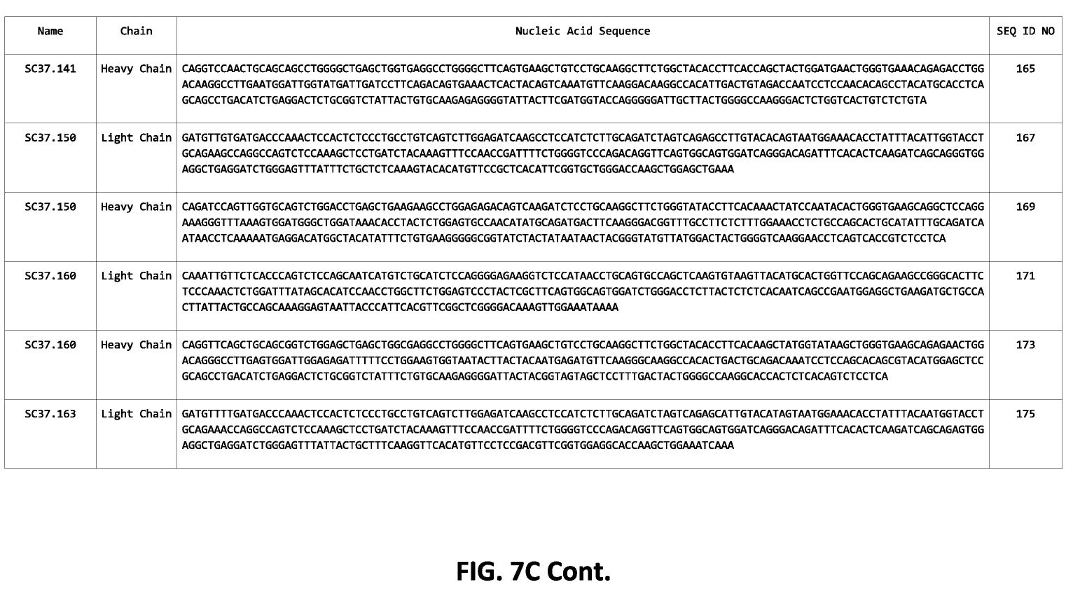

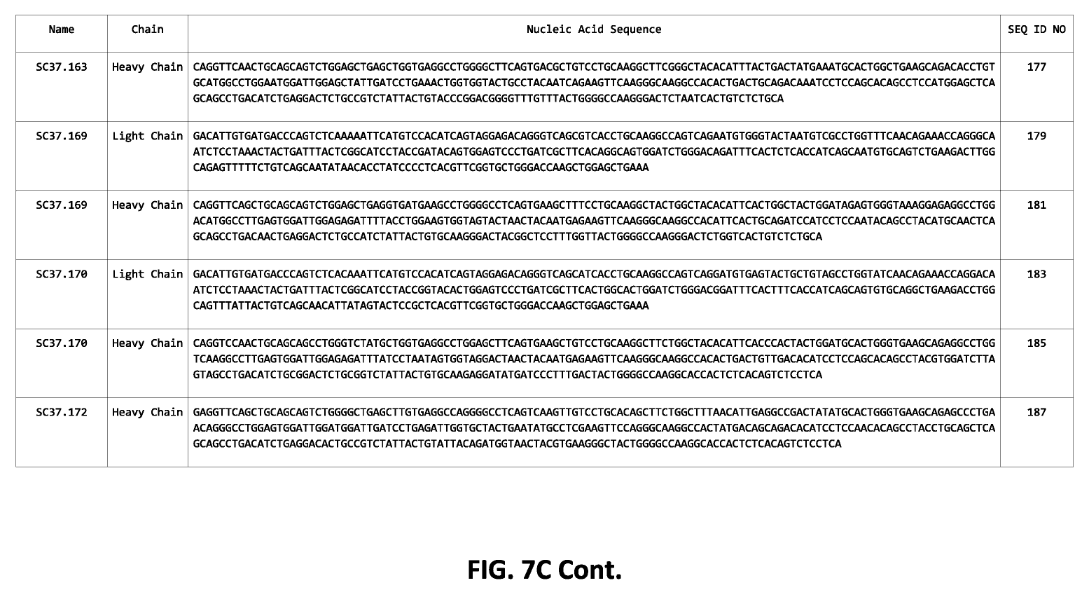

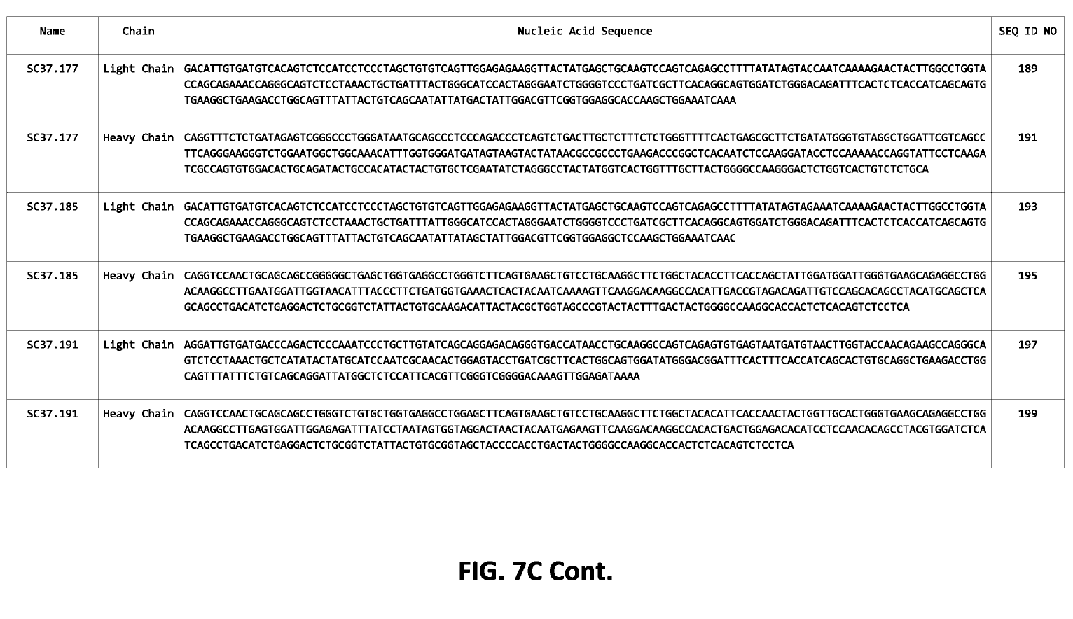

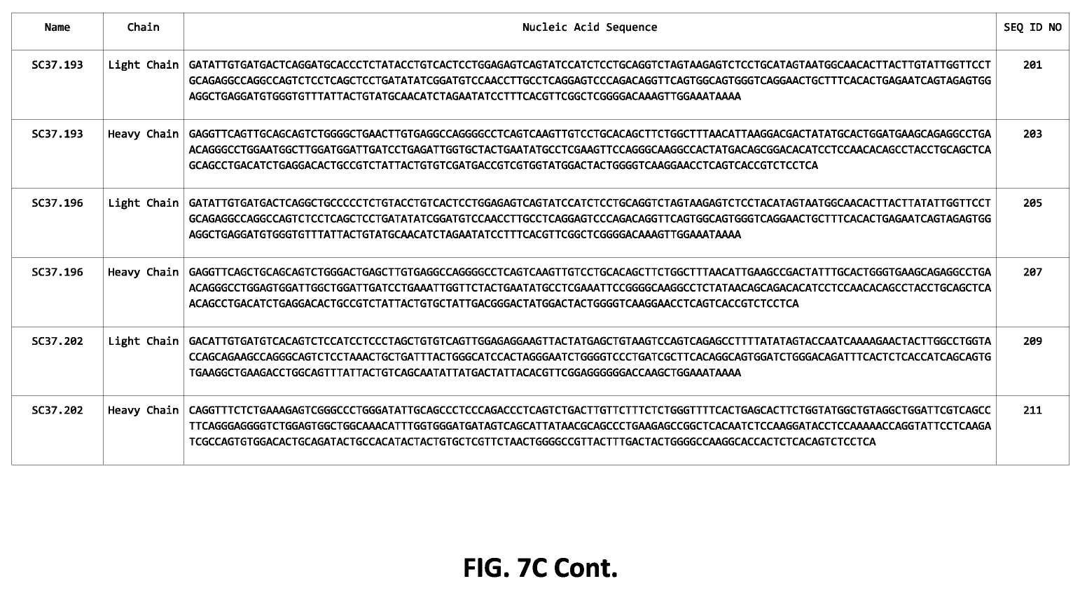

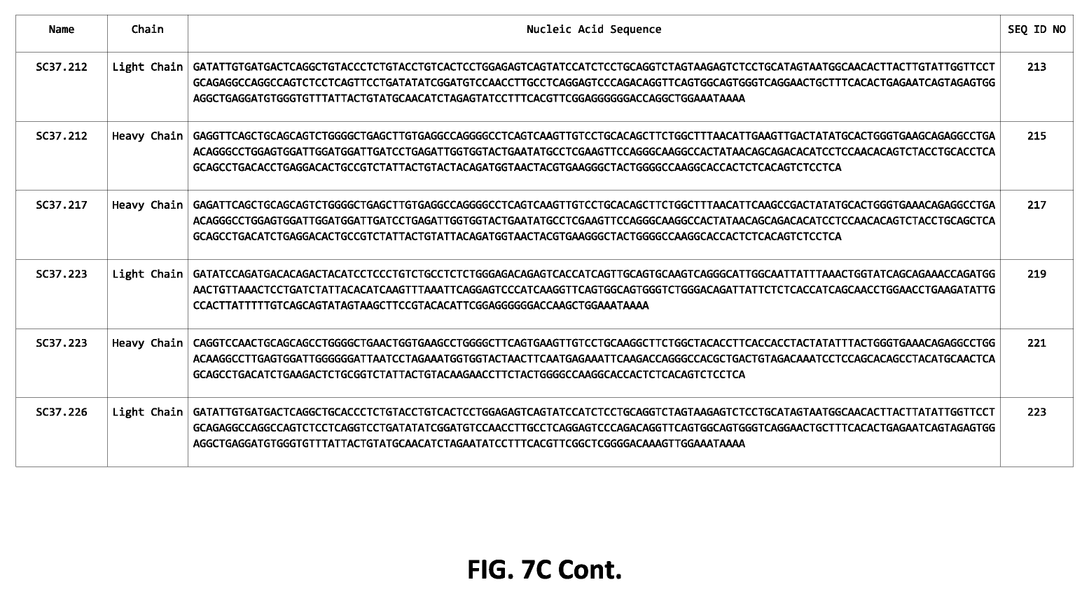

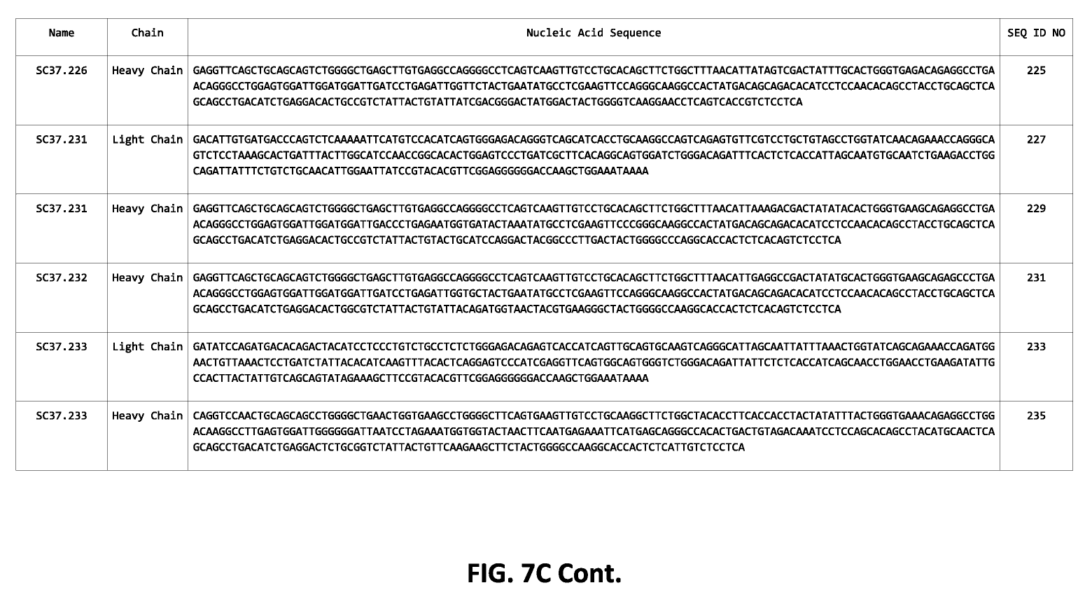

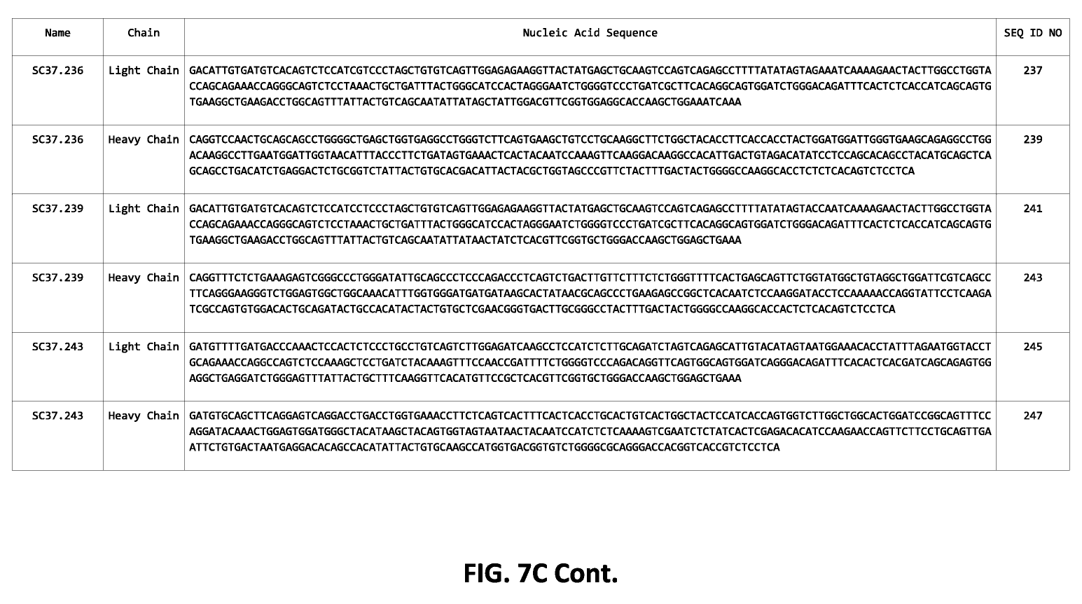

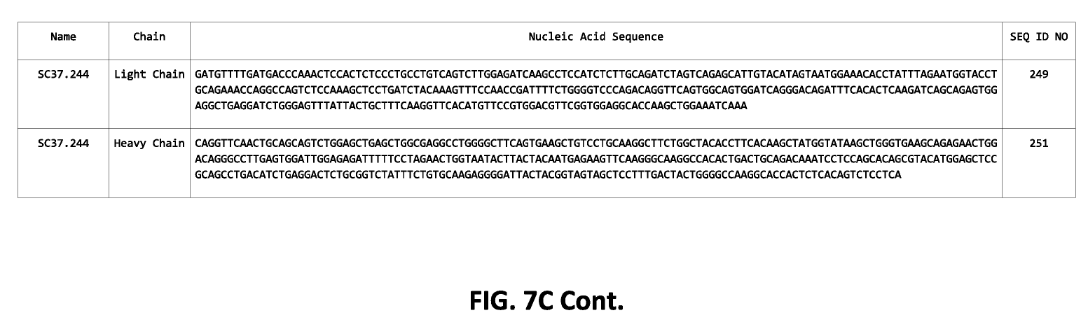

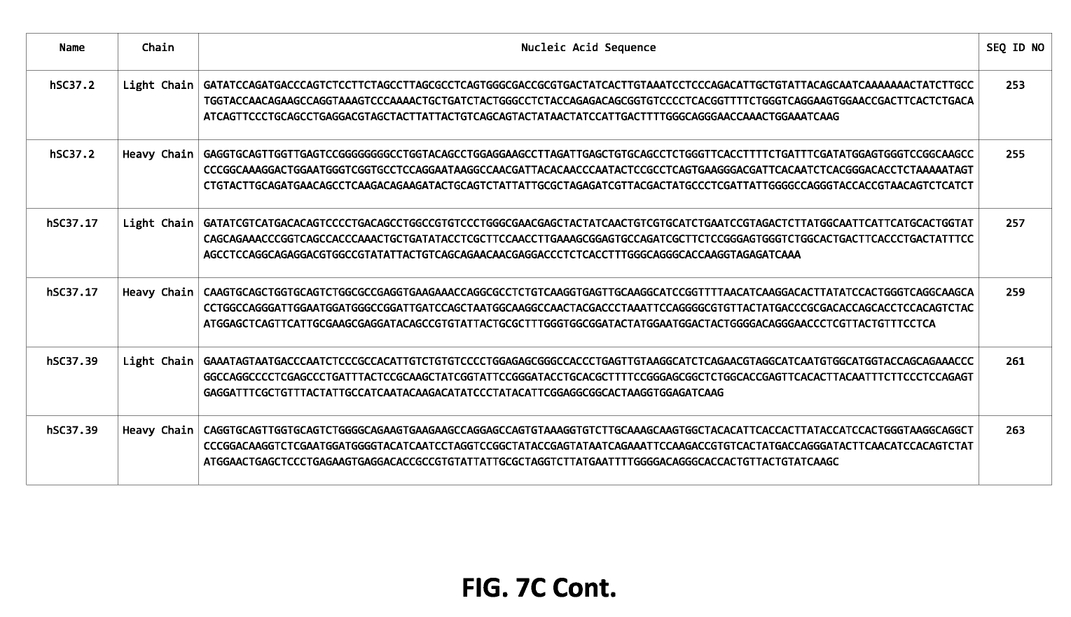

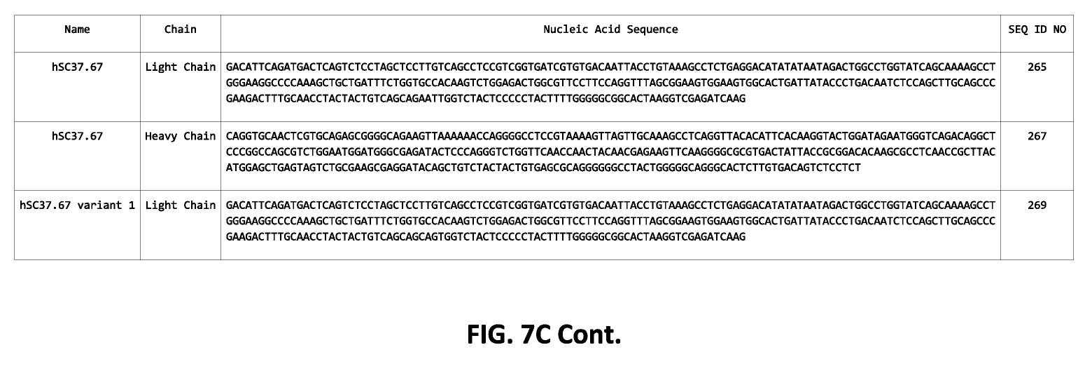

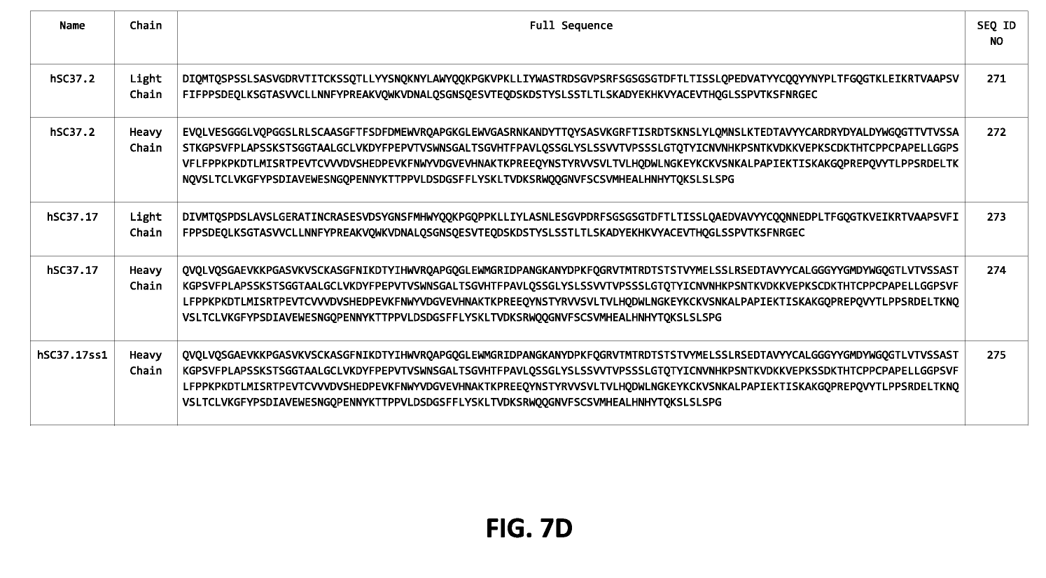

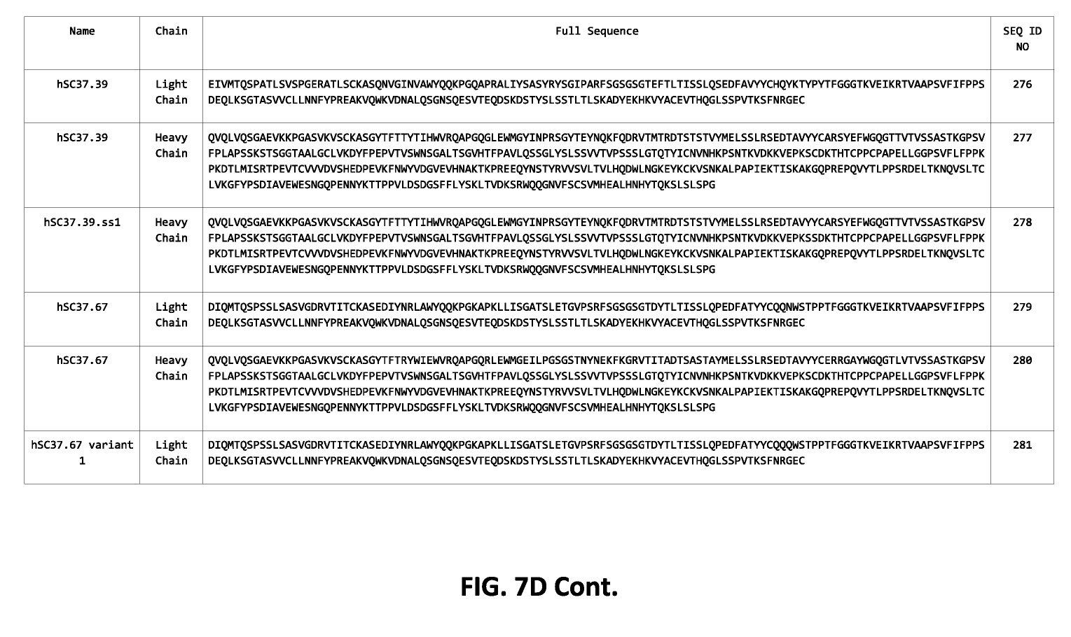

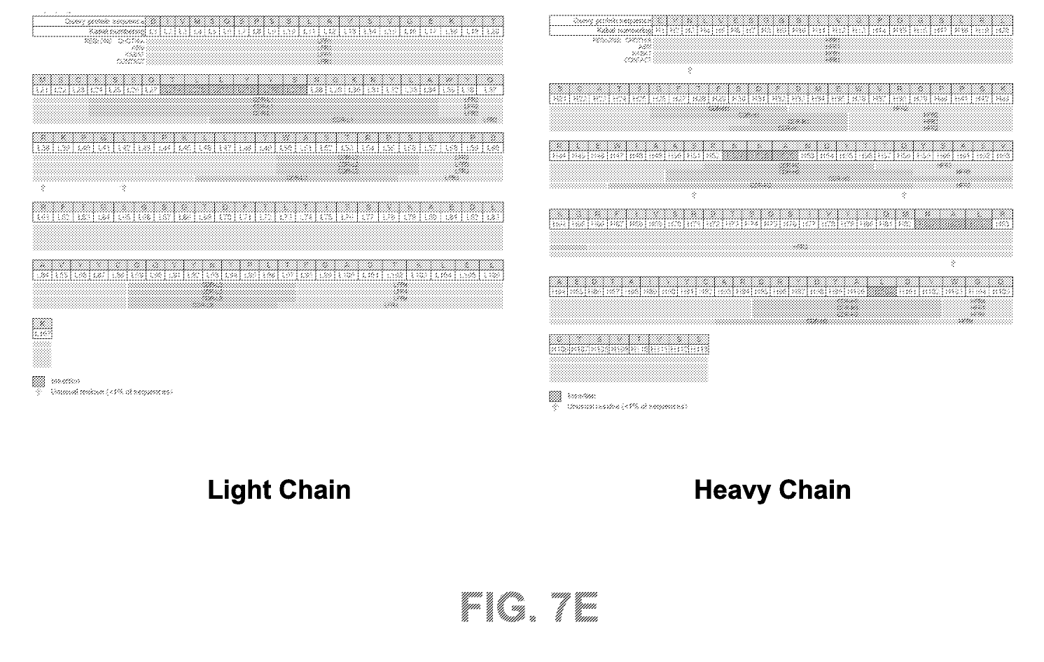

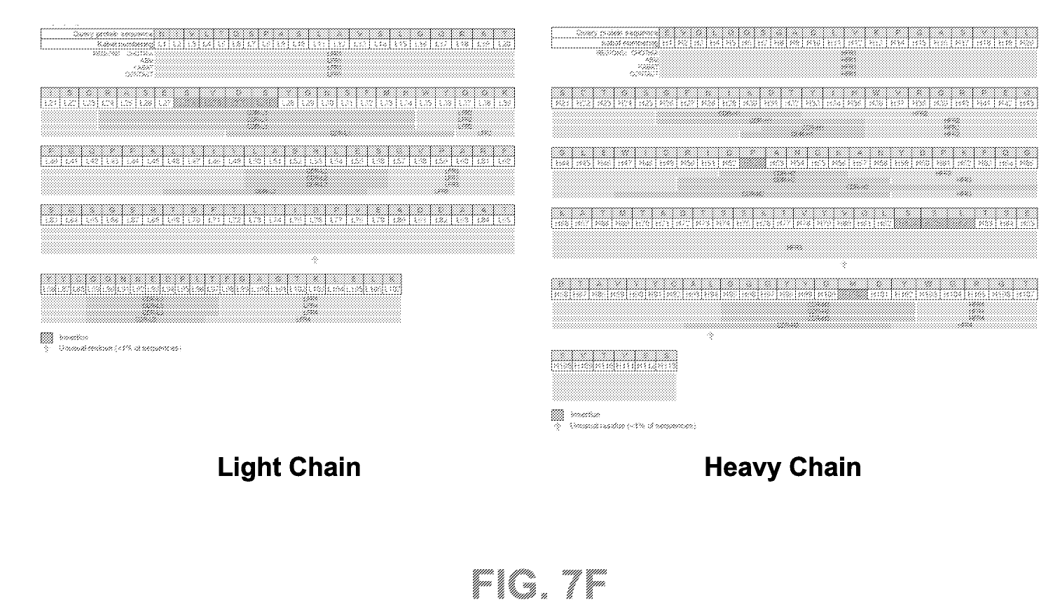

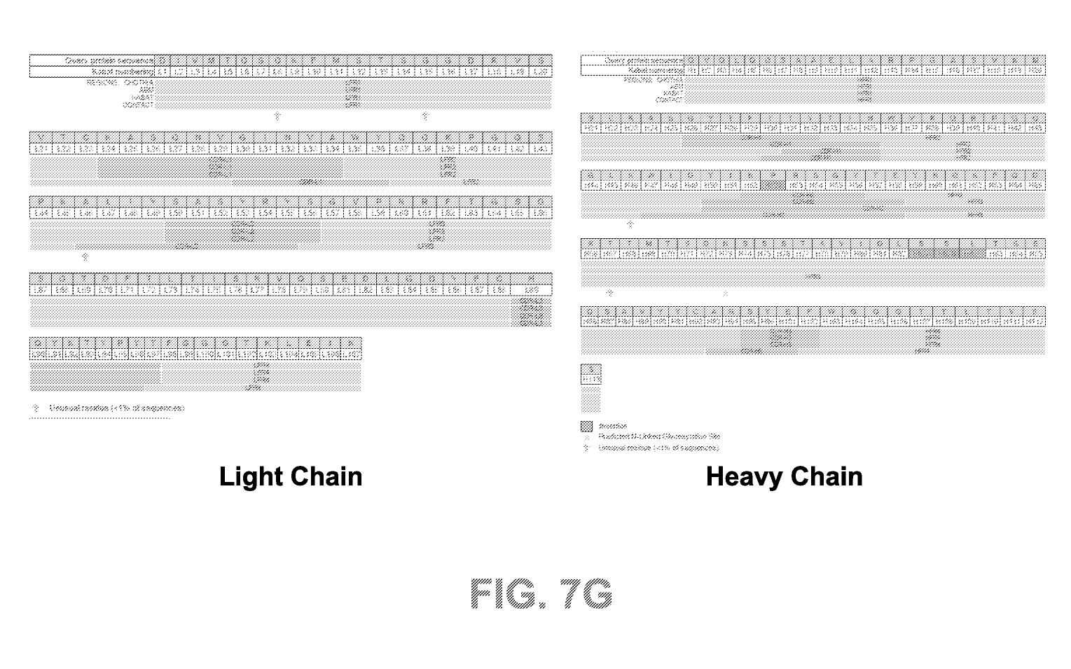

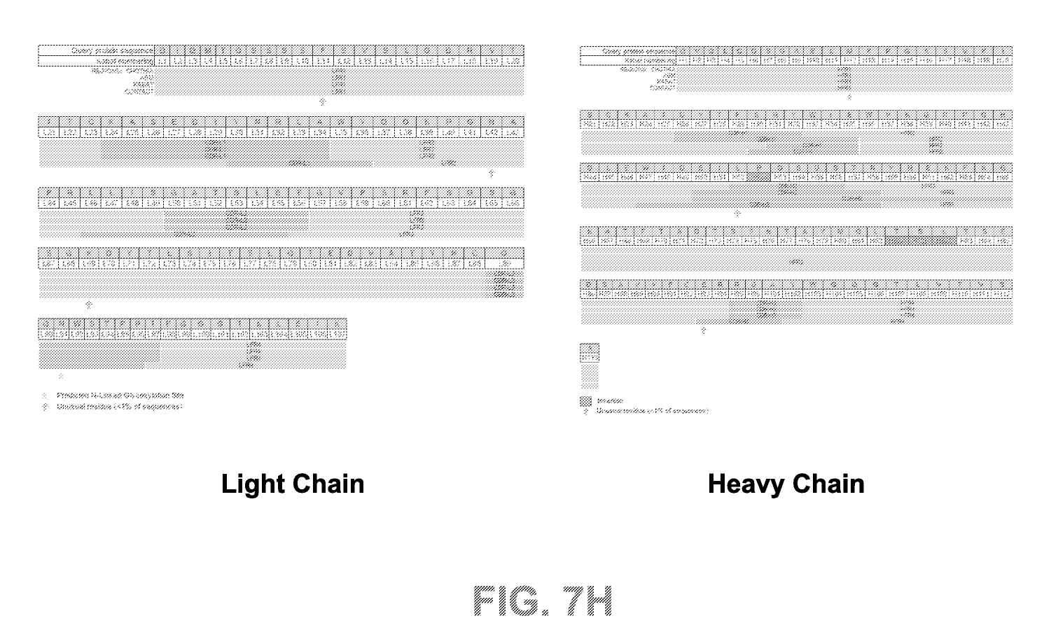

Variable regions and CDRs in an antibody sequence can be identified according to general rules that have been developed in the art (as set out above, such as, for example, the Kabat et al. numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably the sequences are analyzed using the Abysis database, which integrates sequence data from Kabat et al., IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. FIGS. 7E to 7H appended hereto show the results of such analysis in the annotation of several exemplary antibody heavy and light chain variable regions. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of myeloma protein Eu, which reportedly was the first human IgG1 sequenced. The EU index of Edelman is also set forth in Kabat et al., 1991 (supra.). Thus, the terms “EU index as set forth in Kabat” or “EU index of Kabat” or “EU index” in the context of the heavy chain refers to the residue numbering system based on the human IgG1 Eu antibody of Edelman et al. as set forth in Kabat et al., 1991 (supra.) The numbering system used for light chain constant region amino acid sequences is similarly set forth in Kabat et al., (supra.) An exemplary kappa light chain constant region amino acid sequence compatible with the present invention is set forth immediately below:

(SEQ ID NO: 1)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Similarly, an exemplary IgG1 heavy chain constant region amino acid sequence compatible with the present invention is set forth immediately below:

(SEQ ID NO: 2.)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

Those of skill in the art will appreciate that the disclosed constant region sequences may be joined with the disclosed heavy and light chain variable regions using standard molecular biology techniques to provide full-length antibodies that may be used as such or incorporated in the anti-RNF43 ADCs of the invention.

The antibodies or immunoglobulins of the invention may be generated from an antibody that specifically recognizes or associates with any relevant determinant (i.e., RNF43). As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In certain preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a RNF43 protein, or any of its splice variants, isoforms or family members, or specific domains, regions or epitopes thereof. An “antigen”, “immunogenic determinant”, “antigenic determinant” or “immunogen” means any protein or any fragment, region or domain thereof that can stimulate an immune response when introduced into an immunocompetent animal and is recognized by the antibodies produced from the immune response. The presence or absence of the determinants contemplated herein may be used to identify a cell, cell subpopulation or tissue (e.g., tumors, tumorigenic cells or CSCs).

There are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

2. Antibody Generation and Production

Antibodies of the invention can be produced using a variety of methods known in the art.

A. Generation of Polyclonal Antibodies in Host Animals

The generation of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

Any form of antigen, or cells or preparations containing the antigen, can be used to generate an antibody that is specific for a determinant. The term “antigen” is used in a broad sense and may comprise any immunogenic fragment or determinant of the selected target including a single epitope, multiple epitopes, single or multiple domains or the entire extracellular domain (ECD). The antigen may be an isolated full-length protein, a cell surface protein (e.g., immunizing with cells expressing at least a portion of the antigen on their surface), or a soluble protein (e.g., immunizing with only the ECD portion of the protein). The antigen may be produced in a genetically modified cell. Any of the aforementioned antigens may be used alone or in combination with one or more immunogenicity enhancing adjuvants known in the art. The DNA encoding the antigen may be genomic or non-genomic (e.g., cDNA) and may encode at least a portion of the protein, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

B. Monoclonal Antibodies

In selected embodiments, the invention contemplates use of monoclonal antibodies. The term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques including hybridoma techniques, recombinant techniques, phage display technologies, transgenic animals (e.g., a XenoMouse®) or some combination thereof. For example, in preferred embodiments monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following generation of a number of monoclonal antibodies that bind specifically to a determinant, particularly suitable antibodies may be selected through various screening processes, based on, for example, affinity for the determinant or rate of internalization. In particularly preferred embodiments monoclonal antibodies produced as described herein may be used as “source” antibodies and further modified to, for example, to improve affinity for the target, improve its production in cell culture, reduce immunogenicity in vivo, create multispecific constructs, etc. A more detailed description of monoclonal antibody production and screening is set out below and in the appended Examples.

C. Human Antibodies

The antibodies may comprise fully human antibodies. The term “human antibody” refers to an antibody (preferably a monoclonal antibody) which possesses an amino acid sequence that corresponds to that of an antibody produced by a human and/or has been made using any of the techniques for making human antibodies described below.

In one embodiment, recombinant human antibodies may be isolated by screening a recombinant combinatorial antibody library prepared using phage display. In one embodiment, the library is a scFv phage or yeast display library, generated using human VL and VH cDNAs prepared from mRNA isolated from B-cells.

Human antibodies can also be made by introducing human immunoglobulin loci into transgenic animals, e.g., mice in which the endogenous immunoglobulin genes have been partially or completely inactivated and human immunoglobulin genes have been introduced. Upon challenge antibody generation is observed which closely resembles that seen in humans in all respects, including gene rearrangement, assembly and fully human antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, 1995, PMID: 7494109). Alternatively, a human antibody may be prepared via immortalization of human B lymphocytes producing an antibody directed against a target antigen (such B lymphocytes may be recovered from an individual suffering from a neoplastic disorder or who may have been immunized in vitro). See, e.g., Cole et al., Monoclonal Antibodies and Cancer Therapy, Alan R. Liss, p. 77 (1985); Boerner et al., 1991, PMID: 2051030; and U.S. Pat. No. 5,750,373.

D. Derived Antibodies:

Once the source antibodies have been generated, selected and isolated as described above they may be further altered to provide anti-RNF43 antibodies having improved pharmaceutical characteristics. Preferably the source antibodies are modified or altered using known molecular engineering techniques to provide derived antibodies having the desired therapeutic properties.

E. Chimeric and Humanized Antibodies

Selected embodiments of the invention comprise murine antibodies that immunospecifically bind to RNF43 and, for the purposes of the instant disclosure, may be considered “source” antibodies. In selected embodiments, antibodies compatible with the invention can be derived from such “source” antibodies through optional modification of the constant region and/or the antigen binding amino acid sequences of the source antibody. In certain embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor antibody sequence to provide the derivative antibody (e.g. chimeric or humanized antibodies). These “derived” antibodies can be generated using standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the chimeric antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some preferred embodiments chimeric antibodies of the instant invention may comprise all or most of the selected murine heavy and light chain variable regions operably linked to human light and heavy chain constant regions. In other particularly preferred embodiments, anti-RNF43 antibodies may be “derived” from the mouse antibodies disclosed herein.

In other embodiments, the chimeric antibodies of the invention are “CDR grafted” antibodies, where the CDRs (as defined using Kabat, Chothia, McCallum, etc.) are derived from a particular species or belonging to a particular antibody class or subclass, while the remainder of the antibody is derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In particularly preferred embodiments the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention are provided as set forth in Example 10 below.

Various art recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638 each of which is incorporated herein in its entirety. The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The engineered sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

It will be appreciated that the annotated CDRs and framework sequences as provided in the appended Figures are defined as per Kabat et al. using a proprietary Abysis database. However, as discussed herein one skilled in the art could readily identify the CDRs in accordance with the numbering schemes provided by Chothia et al. or MacCallum et al.

F. Site-Specific Antibodies

The antibodies of the instant invention may be engineered to facilitate conjugation to a cytotoxin or other anti-cancer agent (as discussed in more detail below). It is advantageous for the antibody drug conjugate (ADC) preparation to comprise a homogenous population of ADC molecules in terms of the position of the cytotoxin on the antibody and the drug to antibody ratio (DAR). Based on the instant disclosure one skilled in the art could readily fabricate site-specific engineered constructs as described herein. As used herein a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one cytotoxin or other compound conjugated to the unpaired cysteine(s). In certain embodiments the unpaired cysteine residue will comprise an unpaired intrachain residue. In other preferred embodiments the free cysteine residue will comprise an unpaired interchain cysteine residue. The engineered antibody can be of various isotypes, for example, IgG, IgE, IgA or IgD; and within those classes the antibody can be of various subclasses, for example, IgG1, IgG2, IgG3 or IgG4. For IgG1 constructs the light chain of the antibody can comprise either a kappa or lambda isotype each incorporating a C214 that, in preferred embodiments, may be unpaired due to a lack of a C220 residue in the IgG1 heavy chain.

In one embodiment the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an “intrachain cysteine residue” is one naturally paired with another cysteine in the same heavy or light chain. In one embodiment the deleted or substituted interchain cysteine residue is involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In one particularly preferred embodiment an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one particularly preferred embodiment the cysteine at position 214 (C214) of the IgG light chain (kappa or lambda) is deleted or substituted. In another preferred embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-specific constructs provided in Example 17. A summary of these preferred constructs is shown in Table 2 immediately below where all numbering is according to the EU index as set forth in Kabat and WT stands for “wild-type” or native constant region sequences without alterations and delta (A) designates the deletion of an amino acid residue (e.g., C214A indicates that the cysteine at position 214 has been deleted).

TABLE 2

| | Antibody | |
|---|---|---|
| Designation | Component | Alteration |
| ss1 | Heavy Chain | C220S |
| | Light Chain | WT |
| ss2 | Heavy Chain | C220Δ |
| | Light Chain | WT |
| ss3 | Heavy Chain | WT |
| | Light Chain | C214Δ |
| ss4 | Heavy Chain | WT |
| | Light Chain | C214S |

The strategy for generating antibody-drug conjugates with defined sites and stoichiometries of drug loading, as disclosed herein, is broadly applicable to all anti-RNF43 antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention.

G. Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

H. Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

I. Multivalent Constructs

In other embodiments, the antibodies and conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While preferred embodiments only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

In yet other embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

J. Recombinant Production of Antibodies

Antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In a preferred embodiment, the nucleic acid is a cDNA molecule.

Nucleic acids of the invention can be obtained using standard molecular biology techniques. For antibodies expressed by hybridomas (e.g., hybridomas prepared as described further below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (C_{H}1, C_{H}2 and C_{H}3). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth in SEQ ID NO: 1.

Contemplated herein are certain polypeptides (e.g. antigens or antibodies) that exhibit “sequence identity”, sequence similarity” or “sequence homology” to the polypeptides of the invention. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in preferred embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any kind of cellular system which can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under certain conditions. The GS system is discussed in whole or part in connection with EP 0 216 846, EP 0 256 055, EP 0 323 997 and EP 0 338 841 and U.S. Pat. Nos. 5,591,639 and 5,879,936. Another preferred expression system for the development of stable cell lines is the Freedom™ CHO-S Kit (Life Technologies).

Once an antibody of the invention has been produced by recombinant expression or any other of the disclosed techniques, it may be purified or isolated by methods known in the art, meaning that it is identified and separated and/or recovered from its natural environment and separated from contaminants that would interfere with diagnostic or therapeutic uses for the antibody. Isolated antibodies include antibodies in situ within recombinant cells.

These isolated preparations may be purified using various art recognized techniques, such as, for example, ion exchange and size exclusion chromatography, dialysis, diafiltration, and affinity chromatography, particularly Protein A or Protein G affinity chromatography.

K. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### IV. CHARACTERISTICS OF ANTIBODIES

In selected embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted by selecting a particular antigen (e.g., a specific RNF43 isoform) or immunoreactive fragment of the target antigen for inoculation of the animal. In still other embodiments the selected antibodies may be engineered as described above to enhance or refine immunochemical characteristics such as affinity or pharmacokinetics.

1. Neutralizing Antibodies

In selected embodiments the antibodies of the invention may be “antagonists” or “neutralizing” antibodies, meaning that the antibody may associate with a determinant and block or inhibit the activities of said determinant either directly or by preventing association of the determinant with a binding partner such as a ligand or a receptor (e.g., RSPO) thereby interrupting the biological response that otherwise would result from the interaction of the molecules. A neutralizing or antagonist antibody will substantially inhibit binding of the determinant to its ligand or substrate when an excess of antibody reduces the quantity of binding partner bound to the determinant by at least about 20%, 30%, 40%, 50%, 60%, 70%, 80%, 85%, 90%, 95%, 97%, 99% or more as measured, for example, by target molecule activity or in an in vitro competitive binding assay. It will be appreciated that the modified activity may be measured directly using art recognized techniques or may be measured by the impact the altered activity has downstream (e.g., oncogenesis or cell survival).

2. Internalizing Antibodies

There is evidence that a substantial portion of expressed RNF43 protein remains associated with the tumorigenic cell surface, thereby allowing for localization and internalization of the disclosed antibodies or ADCs. In preferred embodiments such antibodies will be associated with, or conjugated to, one or more drugs that kill the cell upon internalization. In particularly preferred embodiments the ADCs of the instant invention will comprise an internalizing site-specific ADC.

As used herein, an antibody that “internalizes” is one that is taken up (along with any cytotoxin) by the cell upon binding to an associated antigen or receptor. For therapeutic applications, internalization will preferably occur in vivo in a subject in need thereof. The number of ADCs internalized may be sufficient to kill an antigen-expressing cell, especially an antigen-expressing cancer stem cell. Depending on the potency of the cytotoxin or ADC as a whole, in some instances, the uptake of a single antibody molecule into the cell is sufficient to kill the target cell to which the antibody binds. For example, certain drugs are so highly potent that the internalization of a few molecules of the toxin conjugated to the antibody is sufficient to kill the tumor cell. Whether an antibody internalizes upon binding to a mammalian cell can be determined by various art-recognized assays including those described in the Examples below. Methods of detecting whether an antibody internalizes into a cell are also described in U.S. Pat. No. 7,619,068.

3. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In preferred embodiments, the selected depleting antibodies will be conjugated to a cytotoxin. Preferably a depleting antibody will be able to kill at least 20%, 30%, 40%, 50%, 60%, 70%, 80%, 85%, 90%, 95%, 97%, or 99% of RNF43-expressing cells in a defined cell population. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

4. Binding Affinity

Disclosed herein are antibodies that have a high binding affinity for a specific determinant e.g. RNF43. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≦10⁻⁷ M. The antibody specifically binds antigen with high affinity when the K_{D} is ≦5×10⁻⁹ M, and with very high affinity when the K_{D} is ≦5×10⁻¹° M. In one embodiment of the invention, the antibody has a K_{D} of ≦10⁻⁹M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≦2×10⁻¹° M. Still other selected embodiments of the invention comprise antibodies that have a K_{D} (k_{off}/kₒₙ) of less than 10⁻⁶ M, less than 5×10⁻⁶M, less than 10⁻⁷M, less than 5×10⁻⁷M, less than 10⁻⁸M, less than 5×10⁻⁸M, less than 10⁻⁹ M, less than 5×10⁻⁹ M, less than 10⁻¹⁰ M, less than 5×10⁻¹⁰ M, less than 10⁻¹¹ M, less than 5×10⁻¹¹M, less than 10⁻¹² M, less than 5×10⁻¹²M, less than 10⁻¹³M, less than 5×10⁻¹³M, less than 10⁻¹⁴M, less than 5×10⁻¹⁴M, less than 10⁻¹⁵M or less than 5×10⁻¹⁵ M.

In certain embodiments, an antibody of the invention that immunospecifically binds to a determinant e.g. RNF43 may have an association rate constant or k_{off} (or k_{d}) rate (antibody+antigen (Ag)^{k}ₒₙ←antibody-Ag) of at least 10⁵ M⁻¹s⁻¹, at least 2×10⁵ M⁻¹S⁻¹, at least 5×10⁵ M⁻¹s⁻¹, at least 10⁶ M⁻s⁻¹, at least 5×10⁶M⁻¹s⁻¹, at least 10⁷ M⁻¹s⁻¹, at least 5×10⁷M⁻¹S⁻¹, or at least 10⁸M⁻¹.

In another embodiment, an antibody of the invention that immunospecifically binds to a determinant e.g. RNF43 may have a disassociation rate constant or k_{off} (or k_{d}) rate (antibody+antigen (Ag)^{k}_{off}←antibody-Ag) of less than 10⁻¹s⁻¹, less than 5×10⁴s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

5. Binning and Epitope Mapping

As used herein, the term “binning” refers to methods used to group antibodies into “bins” based on their antigen binding characteristics and whether they compete with each other. The initial determination of bins may be further refined and confirmed by epitope mapping and other techniques as described herein. However it will be appreciated that empirical assignment of antibodies to individual bins provides information that may be indicative of the therapeutic potential of the disclosed antibodies. As shown in FIG. 5A the disclosed RNF43 antibodies reside in at least six bins labeled A, B, C, D, E and F.

More specifically, one can determine whether a selected reference antibody (or fragment thereof) competes for binding with a second test antibody (i.e., is in the same bin) by using methods known in the art and set forth in the Examples herein. In one embodiment, a reference antibody is associated with RNF43 antigen under saturating conditions and then the ability of a secondary or test antibody to bind to RNF43 is determined using standard immunochemical techniques. If the test antibody is able to substantially bind to RNF43 at the same time as the reference anti-RNF43 antibody, then the secondary or test antibody binds to a different epitope than the primary or reference antibody. However, if the test antibody is not able to substantially bind to RNF43 at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment being tested inhibits specific binding of a reference antibody to a common antigen. Typically, such an assay involves the use of purified antigen (e.g., RNF43 or a domain or fragment thereof) bound to a solid surface or cells, an unlabeled test antibody and a labeled reference antibody. Competitive inhibition is measured by determining the amount of label bound to the solid surface or cells in the presence of the test antibody. Usually the test antibody is present in excess and/or allowed to bind first. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody (i.e., an anti-RNF43 antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In preferred embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

“Surface plasmon resonance,” refers to an optical phenomenon that allows for the analysis of real-time specific interactions by detection of alterations in protein concentrations within a biosensor matrix.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In preferred embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which the antibodies in a bin bind. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds. The term “epitope” is used in its common biochemical sense and refers to that portion of the target antigen capable of being recognized and specifically bound by a particular antibody. In certain embodiments, epitopes or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. In certain embodiments, an antibody is said to specifically bind an antigen when it preferentially recognizes its target antigen in a complex mixture of proteins and/or macromolecules.

When the antigen is a polypeptide such as RNF43, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. An antibody epitope typically includes at least 3, and more usually, at least 5 or 8-10 amino acids in a unique spatial conformation. Methods of epitope determination or “epitope mapping” are well known in the art and may be used in conjunction with the instant disclosure to identify epitopes on RNF43 bound by the disclosed antibodies.

Compatible epitope mapping techniques include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Other compatible methods comprise yeast display methods. In other embodiments Modification-Assisted Profiling (MAP), also known as Antigen Structure-based Antibody Profiling (ASAP) provides a method that categorizes large numbers of monoclonal antibodies directed against the same antigen according to the similarities of the binding profile of each antibody to chemically or enzymatically modified antigen surfaces (U.S.P.N. 2004/0101920). This technology allows rapid filtering of genetically identical antibodies, such that characterization can be focused on genetically distinct antibodies. It will be appreciated that MAP may be used to sort the anti-RNF43 antibodies of the invention into groups of antibodies binding different epitopes

Once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

### V. ANTIBODY CONJUGATES

In certain preferred embodiments the antibodies of the invention may be conjugated with pharmaceutically active or diagnostic moieties to form an “antibody drug conjugate” (ADC) or “antibody conjugate”. The term “conjugate” is used broadly and means the covalent or non-covalent association of any pharmaceutically active or diagnostic moiety with an antibody of the instant invention regardless of the method of association. In certain embodiments the association is effected through a lysine or cysteine residue of the antibody. In particularly preferred embodiments the pharmaceutically active or diagnostic moieties may be conjugated to the antibody via one or more site-specific free cysteine(s). The disclosed ADCs may be used for therapeutic and diagnostic purposes.

The ADCs of the instant invention may be used to deliver cytotoxins or other payloads to the target location (e.g., tumorigenic cells and/or cells expressing RNF43). As used herein the terms “drug” or “warhead” may be used interchangeably and will mean a biologically active or detectable molecule or compound, including anti-cancer agents as described below. A “payload” may comprise a drug or warhead in combination with an optional linker compound. The warhead on the conjugate may comprise peptides, proteins, or prodrugs which are metabolized to an active agent in vivo, polymers, nucleic acid molecules, small molecules, binding agents, mimetic agents, synthetic drugs, inorganic molecules, organic molecules and radioisotopes. In an advantageous embodiment, the disclosed ADCs will direct the bound payload to the target site in a relatively unreactive, non-toxic state before releasing and activating the payload. This targeted release of the payload is preferably achieved through stable conjugation of the payloads (e.g., via one or more cysteines on the antibody) and the relatively homogeneous composition of the ADC preparations which minimize over-conjugated toxic species. Coupled with drug linkers that are designed to largely release the payload once it has been delivered to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index.

It will be appreciated that, while preferred embodiments of the invention comprise payloads of therapeutic moieties (e.g., cytotoxins), other payloads such as diagnostic agents and biocompatible modifiers may benefit from the targeted release provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to, the antibody and exhibit various stoichiometric molar ratios depending, at least in part, on the method used to effect the conjugation. The conjugates of the instant invention may be represented by the formula:

Ab-[L-D]n or a pharmaceutically acceptable salt thereof wherein
a) Ab comprises an anti-RNF43 antibody;
b) L comprises an optional linker;
c) D comprises a drug; and
d) n is an integer from 1 to 20.

Those of skill in the art will appreciate that conjugates according to the aforementioned formula may be fabricated using a number of different linkers and drugs and that conjugation methodology will vary depending on the selection of components. As such, any drug or drug linker compound that associates with a reactive residue (e.g., cysteine or lysine) of the disclosed antibodies are compatible with the teachings herein. Similarly, any reaction conditions that allow for site-specific conjugation of the selected drug to an antibody are within the scope of the present invention. Notwithstanding the foregoing, particularly preferred embodiments of the instant invention comprise selective conjugation of the drug or drug linker to free cysteines using stabilization agents in combination with mild reducing agents as described herein. Such reaction conditions tend to provide more homogeneous preparations with less non-specific conjugation and contaminants and correspondingly less toxicity. Exemplary payloads compatible with the teachings herein are set forth below.

1. Therapeutic Agents

The antibodies of the invention may be conjugated, linked or fused to or otherwise associated with a pharmaceutically active moiety which is a therapeutic moiety or a drug such as an anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Preferred exemplary anti-cancer agents (including homologs and derivatives thereof) comprise 1-dehydrotestosterone, anthramycins, actinomycin D, bleomycin, calicheamicin, colchicin, cyclophosphamide, cytochalasin B, dactinomycin (formerly actinomycin), dihydroxy anthracin, dione, emetine, epirubicin, ethidium bromide, etoposide, glucocorticoids, gramicidin D, lidocaine, maytansinoids such as DM-1 and DM-4 (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In certain selected embodiments the disclosed antibodies will be conjugated to one or more calicheamicin(s). As used herein the term “calicheamicin” shall be held to mean any one of calicheamicin γ1I, calicheamicin β1Br, calicheamicin γ1Br, calicheamicin α2I, calicheamicin α3I, calicheamicin β1i and calicheamicin δ1 along with n-acetyl derivatives and sulfide analogs thereof. In certain embodiments the calicheamicin component of the disclosed antibody drug conjugates will comprise N-acetyl Calicheamicin γ1^{I}.

In one embodiment the antibodies of the instant invention may be associated with anti-CD3 binding molecules to recruit cytotoxic T-cells and have them target tumorigenic cells (BiTE technology; see e.g., Fuhrmann et. al. (2010) Annual Meeting of AACR Abstract No. 5625).

In further embodiments ADCs of the invention may comprise therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I) carbon (¹⁴C), copper (⁶²Cu, ⁶⁴Cu, ⁶⁷Cu), sulfur (³⁵S), tritium (³H), indium (¹¹⁵In, ¹¹³In, ¹¹²In, ¹¹¹In), bismuth (²¹²Bi, ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Sn, ²²⁵Ac, ⁷⁶Br, and ²¹¹At. Other radionuclides are also available as diagnostic and therapeutic agents, especially those in the energy range of 60 to 4,000 keV.

In certain preferred embodiments, the ADCs of the invention may comprise PBDs and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157, and PCT filings WO2011/130613, WO2011/128650, WO2011/130616 and WO2014/057074.

Antibodies of the present invention may also be conjugated to biological response modifiers. For example, in particularly preferred embodiments the drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-β or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other preferred embodiments, the antibodies of the invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis, analysis and/or detection can be accomplished by coupling the antibody to detectable substances including, but not limited to, various enzymes comprising for example horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I), to carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabeled or conjugated to specific radioisotopes. In such embodiments appropriate detection methodology is well known in the art and readily available from numerous commercial sources.

In other embodiments the antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In preferred embodiments, the marker comprises a histidine tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments the antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

4. Linker Compounds

Numerous linker compounds can be used to conjugate the antibodies of the invention to the relevant warhead. The linkers merely need to covalently bind with the reactive residue on the antibody (preferably a cysteine or lysine) and the selected drug compound. Accordingly, any linker that reacts with the selected antibody residue and may be used to provide the relatively stable conjugates (site-specific or otherwise) of the instant invention is compatible with the teachings herein.

Numerous compatible linkers can advantageously bind to reduced cysteines and lysines, which are nucleophilic. Conjugation reactions involving reduced cysteines and lysines include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in preferred embodiments the use of selective reduction and site-specific antibodies as set forth herein in Example 13 may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

In preferred embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the antibody remains linked to the drug moiety. While the linkers are stable outside the target cell they are designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety (including, in some cases, any bystander effects). The stability of the ADC may be measured by standard analytical techniques such as HPLC/UPLC, mass spectroscopy, HPLC, and the separation/analysis techniques LC/MS and LC/MS/MS. As set forth above covalent attachment of the antibody and the drug moiety requires the linker to have two reactive functional groups, i.e. bivalency in a reactive sense. Bivalent linker reagents which are useful to attach two or more functional or biologically active moieties, such as MMAE and site-specific antibodies are known, and methods have been described to provide their resulting conjugates.

Linkers compatible with the present invention may broadly be classified as cleavable and non-cleavable linkers. Cleavable linkers, which may include acid-labile linkers, protease cleavable linkers and disulfide linkers, are preferably internalized into the target cell and are cleaved in the endosomal-lysosomal pathway inside the cell. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethyleneglycol or alkyl spacers liberate toxic payloads during lysosomal degradation of the ADC within the target cell. In some respects the selection of linker will depend on the particular drug used in the conjugate, the particular indication and the antibody target.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells. Exemplary peptidyl linkers that are cleavable by the thiol-dependent protease Cathepsin-B are peptides comprising Phe-Leu since cathepsin-B has been found to be highly expressed in cancerous tissue. Other examples of such linkers are described, for example, in U.S. Pat. No. 6,214,345. In a specific preferred embodiment, the peptidyl linker cleavable by an intracellular protease is a Val-Cit linker, a Val-Ala linker or a Phe-Lys linker such as is described in U.S. Pat. No. 6,214,345. One advantage of using intracellular proteolytic release of the therapeutic agent is that the agent is typically attenuated when conjugated and the serum stabilities of the conjugates are typically high.

In other embodiments, the cleavable linker is pH-sensitive. Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable at below pH 5.5 or 5.0, the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In particularly preferred embodiments (set forth in U.S.P.N. 2011/0256157) compatible peptidyl linkers will comprise:

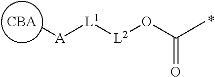

where the asterisk indicates the point of attachment to the drug, CBA is the anti-RNF43 antibody, L¹ is a linker, A is a connecting group (optionally comprising a spacer) connecting L¹ to a reactive residue on the antibody, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidizing conditions may also find use in the present invention.

L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from: -Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala-.

In one embodiment, L² is present and together with —C(═O)O— forms a self-immolative linker. In one embodiment, L² is a substrate for enzymatic activity, thereby allowing release of the drug.

In one embodiment, where L¹ is cleavable by the action of an enzyme and L² is present, the enzyme cleaves the bond between L¹ and L².

L¹ and L², where present, may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

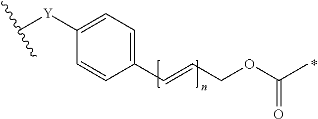

where the asterisk indicates the point of attachment to the drug or cytotoxic agent (optionally through a spacer) position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents as described herein.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In another particularly preferred embodiments the linker may include a self-immolative linker and the dipeptide together form the group —NH-Val-Ala-CO—NH-PABC-, which is illustrated below:

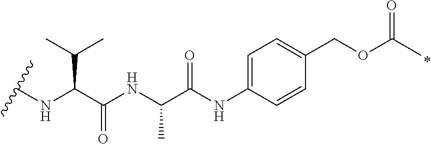 [SMILES: *N[C@H](C(=O)N[C@@H](C)C(=O)Nc1ccc(COC(C)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide the self-immolative linker will allow for clean release of the protected compound (i.e., the cytotoxin) when a remote site is activated, proceeding along the lines shown below:

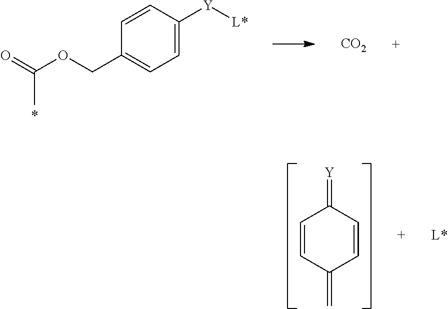

where L* is the activated form of the remaining portion of the linker comprising the now cleaved peptidyl unit. The clean release of the drug ensures they will maintain the desired toxic activity. In another preferred embodiment the linker will comprise —NH-Val-Cit-CO—NH-PABC-.

In one embodiment, A is a covalent bond. Thus, L¹ and the antibody are directly connected. For example, where L¹ comprises a contiguous amino acid sequence, the N-terminus of the sequence may connect directly to the antibody residue.

In another embodiment, A is a spacer group. Thus, L¹ and the antibody are indirectly connected.

L¹ and A may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below the drug linkers of the instant invention will preferably be linked to reactive thiol nucleophiles on cysteines, including free cysteines. To this end the cysteines of the antibodies may be made reactive for conjugation with linker reagents by treatment with various reducing agent such as DTT or TCEP or mild reducing agents as set forth herein. In other embodiments the drug linkers of the instant invention will preferably be linked to a lysine.

Preferably, the linker contains an electrophilic functional group for reaction with a nucleophilic functional group on the antibody. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones, carboxyl, and, some of which are exemplified as follows:

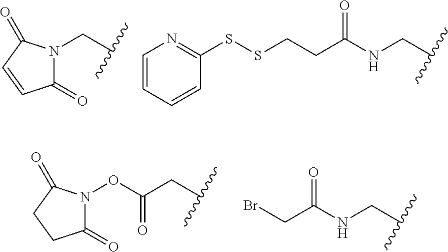

In particularly preferred embodiments the connection between a site-specific antibody and the drug-linker moiety is through a thiol residue of a free cysteine of the site specific antibody and a terminal maleimide group of present on the linker. In such embodiments, the connection between the antibody and the drug-linker is:

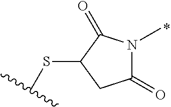 [SMILES: *SC1CC(=O)N(C)C1=O]

where the asterisk indicates the point of attachment to the remaining portion of drug-linker and the wavy line indicates the point of attachment to the remaining portion of the antibody. In this embodiment, the S atom is preferably derived from a site-specific free cysteine. With regard to other compatible linkers the binding moiety comprises a terminal iodoacetamide that may be reacted with activated residues to provide the desired conjugate. In any event one skilled in the art could readily conjugate each of the disclosed drug-linker compounds with a compatible anti-RNF43 antibody (e.g., a site specific antibody) in view of the instant disclosure.

5. Conjugation

It will be appreciated that a number of art recognized different reactions may be used to attach the drug moiety and/or linker to the selected antibody. For example, various reactions exploiting sulfhydryl groups of cysteines may be employed to conjugate the desired moiety. Particularly preferred embodiments will comprise conjugation of antibodies comprising one or more free cysteines as discussed in detail below. In other embodiments ADCs of the instant invention may be generated through conjugation of drugs to solvent-exposed amino groups of lysine residues present in the selected antibody. Still other embodiments comprise activation of the N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles, such as maleimides and iodoacetamides. Generally reagents for such conjugations may react directly with a cysteine thiol of a cysteine to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug-linker intermediate, via a covalent bond, followed by reaction with a cysteine group of a protein of the invention. As will be apparent to the skilled artisan from the foregoing, bifunctional linkers are useful in the present invention. For example, the bifunctional linker may comprise a thiol modification group for covalent linkage to the cysteine residue(s) and at least one attachment moiety (e.g., a second thiol modification moiety) for covalent or non-covalent linkage to the compound.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut's reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Preferred conjugation reagents include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In particularly preferred embodiments reactive thiol groups may be introduced into the selected antibody (or fragment thereof) by introducing one, two, three, four, or more free cysteine residues (e.g., preparing antibodies comprising one or more free non-native cysteine amino acid residues). Such site-specific antibodies or engineered antibodies, allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and direction of the drug-linker to the same. The conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

The site-specific constructs present free cysteine(s), which when reduced comprise thiol groups that are nucleophilic and capable of reacting to form covalent bonds with electrophilic groups on linker moieties such as those disclosed above. Preferred antibodies of the instant invention will have reducible unpaired interchain or intrachain cysteines, i.e. cysteines providing such nucleophilic groups. Thus, in certain embodiments the reaction of free sulfhydryl groups of the reduced unpaired cysteines and the terminal maleimido or haloacetamide groups of the disclosed drug-linkers will provide the desired conjugation. In such cases the free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are compatible it will be appreciated that conjugation of the site-specific antibodies may be effected using various reactions, conditions and reagents known to those skilled in the art.

In addition it has been found that the free cysteines of engineered antibodies, whether introduced or derived from a native interchain or intrachain disulfide bond, may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this may be affected by certain reducing agents. In other preferred embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced with a cytotoxin as described herein. In this respect the use of such stabilizing agents in combination with selected reducing agents can markedly improve the efficiency of site-specific conjugation as determined by extent of conjugation on the heavy and light antibody chains and DAR distribution of the preparation.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site. Such agents (e.g., certain amino acids) are known to form salt bridges (via hydrogen bonding and electrostatic interactions) and may modulate protein-protein interactions in such a way as to impart a stabilizing effect that may cause favorable conformation changes and/or may reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to this considerably reduces the levels of non-specific conjugation and corresponding impurities in conjugate preparations fabricated as set forth herein.

In selected embodiments stabilizing agents compatible with the present invention will generally comprise compounds with at least one moiety having a basic pKa. In certain embodiments the moiety will comprise a primary amine while in other preferred embodiments the amine moiety will comprise a secondary amine. In still other preferred embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In particularly preferred embodiments, compatible stabilizing agents may comprise arginine, lysine, proline and cysteine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other preferred embodiments will comprise stabilizing agents where the amine moiety will have a pKa of greater than about 9.5 while certain other embodiments will comprise stabilizing agents exhibiting at least one amine moiety having a pKa of greater than about 10.0. In still other preferred embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the engineered antibody native disulfide bonds. Under such conditions, provided by the combination of selected stabilizing and reducing agents, the activated drug linker is largely limited to binding to the desired free site-specific cysteine site. Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In preferred embodiments mild reducing agents may comprise compounds having one or more free thiols while in particularly preferred embodiments mild reducing agents will comprise compounds having a single free thiol. Non-limiting examples of reducing agents compatible with the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site (in this invention the free cysteine on the c-terminus of the light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

6. DAR Distribution and Purification

One of the advantages of conjugation with site specific antibodies of the present invention is the ability to generate relatively homogeneous ADC preparations comprising a narrow DAR distribution. In this regard the disclosed constructs and/or selective conjugation provides for homogeneity of the ADC species within a sample in terms of the stoichiometric ratio between the drug and the engineered antibody. As briefly discussed above the term “drug to antibody ratio” or “DAR” refers to the molar ratio of drug to antibody. In some embodiments a conjugate preparation may be substantially homogeneous with respect to its DAR distribution, meaning that within the preparation is a predominant species of site-specific ADC with a particular DAR (e.g., a DAR of 2 or 4) that is also uniform with respect to the site of loading (i.e., on the free cysteines). In certain embodiments of the invention it is possible to achieve the desired homogeneity through the use of site-specific antibodies and/or selective reduction and conjugation. In other preferred embodiments the desired homogeneity may be achieved through the use of site-specific constructs in combination with selective reduction. In yet other particularly preferred embodiments the preparations may be further purified using analytical or preparative chromatography techniques. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

The disclosed ADCs and preparations thereof may comprise drug and antibody moieties in various stoichiometric molar ratios depending on the configuration of the antibody and, at least in part, on the method used to effect conjugation. In certain embodiments the drug loading per ADC may comprise from 1-20 warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In certain preferred embodiments the ADCs will comprise from 1 to 8 warheads.

While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >6, may cause aggregation, insolubility, toxicity, or loss of cellular permeability of certain antibody-drug conjugates. In view of such concerns practical drug loading provided by the instant invention preferably ranges from 1 to 8 drugs per conjugate, i.e. where 1, 2, 3, 4, 5, 6, 7, or 8 drugs are covalently attached to each antibody (e.g., for IgG1, other antibodies may have different loading capacity depending the number of disulfide bonds). Preferably the DAR of compositions of the instant invention will be approximately 2, 4 or 6 and in particularly preferred embodiments the DAR will comprise approximately 2.

Despite the relatively high level of homogeneity provided by the instant invention the disclosed compositions actually comprise a mixture of conjugates with a range of drugs compounds, from 1 to 8 (in the case of a IgG1). As such, the disclosed ADC compositions include mixtures of conjugates where most of the constituent antibodies are covalently linked to one or more drug moieties and (despite the conjugate specificity of selective reduction) where the drug moieties may be attached to the antibody by various thiol groups. That is, following conjugation ADC compositions of the invention will comprise a mixture of conjugates with different drug loads (e.g., from 1 to 8 drugs per IgG1 antibody) at various concentrations (along with certain reaction contaminants primarily caused by free cysteine cross reactivity). Using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting. Thus, in certain preferred embodiments the present invention will comprise compositions having an average DAR of 1, 2, 3, 4, 5, 6, 7 or 8 each +/−0.5. In other preferred embodiments the present invention will comprise an average DAR of 2, 4, 6 or 8+/−0.5. Finally, in selected preferred embodiments the present invention will comprise an average DAR of 2+/−0.5. It will be appreciated that the range or deviation may be less than 0.4 in certain preferred embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other preferred embodiments the ADC composition will comprise an average DAR of 2, 4, 6 or 8 each +/−0.4 with relatively low levels (<30%) of non-predominant ADC species. In particularly preferred embodiments the ADC composition will comprise an average DAR of 2+/−0.4 with relatively low levels (<30%) of non-predominant ADC species. In yet other embodiments the predominant ADC species (e.g., DAR of 2) will be present at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against other DAR species.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### VI. DIAGNOSTICS AND SCREENING

1. Diagnostics

The invention provides in vitro or in vivo methods for detecting, diagnosing or monitoring proliferative disorders and methods of screening cells from a patient to identify tumor cells including tumorigenic cells (e.g. CSCs). Such methods include identifying an individual having cancer for treatment or monitoring progression of a cancer comprising contacting the patient or a sample obtained from a patient (i.e. either in vivo or in vitro) with an antibody as described herein and detecting presence or absence, or level of association, of the antibody to bound or free target molecules in the sample. In some embodiments the antibody will comprise a detectable label or reporter molecule as described herein.

In some embodiments, the association of the antibody with particular cells in the sample can denote that the sample expresses the protein that is the target of the antibodies of the invention (e.g. RNF43), thereby indicating that the individual having cancer may be effectively treated with an antibody or antibody drug conjugate as described herein. Samples can be analyzed using numerous assays, for example radioimmunoassays, enzyme immunoassays (e.g. ELISA), competitive-binding assays, fluorescent immunoassays, immunoblot assays, Western Blot analysis and flow cytometry assays. Compatible in vivo theragnostic or diagnostic assays can comprise art recognized imaging or monitoring techniques, for example, magnetic resonance imaging, computerized tomography (e.g. CAT scan), positron tomography (e.g., PET scan), radiography, ultrasound, etc., as would be known by those skilled in the art.

In another embodiment, the invention provides a method of analyzing cancer progression and/or pathogenesis in vivo. In another embodiment, analysis of cancer progression and/or pathogenesis in vivo comprises determining the extent of tumor progression. In a further embodiment, analysis comprises the identification of the tumor. In another embodiment, analysis of tumor progression is performed on the primary tumor. In another embodiment, analysis is performed over time depending on the type of cancer as known to one skilled in the art. In another embodiment, further analysis of secondary tumors originating from metastasizing cells of the primary tumor is analyzed in-vivo. In another embodiment, the size and shape of secondary tumors are analyzed. In some embodiments, further ex vivo analysis is performed.

In another embodiment, the invention provides a method of analyzing cancer progression and/or pathogenesis in vivo including determining cell metastasis or detecting and quantifying the level of circulating tumor cells. In yet another embodiment, analysis of cell metastasis comprises determination of progressive growth of cells at a site that is discontinuous from the primary tumor. In another embodiment, the site of cell metastasis analysis comprises the route of neoplastic spread. In some embodiment, cells can disperse via blood vasculature, lymphatics, within body cavities or combinations thereof. In another embodiment, cell metastasis analysis is performed in view of cell migration, dissemination, extravasation, proliferation or combinations thereof.

Accordingly, in one embodiment the antibodies of the instant invention may be used to detect and quantify RNF43 levels in a patient sample (e.g., plasma or blood) which may, in turn, be used to detect, diagnose or monitor RNF43 associated disorders including proliferative disorders. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In certain embodiments of the invention, the tumorigenic cells in a subject or a sample from a subject may be assessed or characterized using the disclosed antibodies prior to therapy or regimen to establish a baseline. In other examples, the tumorigenic cells can be assessed from a sample that is derived from a subject that was treated. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the tumorigenic cells are assessed or characterized after a certain number of doses (e.g., after 1, 2, 3, 4, 5, 10, 20, 30 or more doses of a therapy). In other examples, the tumorigenic cells are characterized or assessed after 1 week, 2 weeks, 3 weeks, 1 month, 6 weeks, 2 months, 1 year, 2 years, 3 years, 4 years or more after receiving one or more therapies.

In another aspect, and as discussed in more detail below, the present invention provides kits for detecting, monitoring or diagnosing a hyperproliferative disorder, identifying individual having such a disorder for possible treatment or monitoring progression (or regression) of the disorder in a patient, wherein the kit comprises an antibody as described herein, and reagents for detecting the impact of the antibody on a sample.

Yet another aspect of the instant invention comprises the use of labeled anti-RNF43 antibodies for use in immunohistochemistry (IHC). In this respect IHC may be used as a diagnostic tool to aid in the diagnosis of various proliferative disorders and to monitor the potential response to treatments including anti-RNF43 antibody therapy. Compatible diagnostic assays may be performed on tissues that have been chemically fixed (including but not limited to: formaldehyde, gluteraldehyde, osmium tetroxide, potassium dichromate, acetic acid, alcohols, zinc salts, mercuric chloride, chromium tetroxide and picric acid) and embedded (including but not limited to: glycol methacrylate, paraffin and resins) or preserved via freezing. Such assays can be used to guide treatment decisions and determine dosing regimens and timing.

2. Screening

In certain embodiments, the antibodies can be used to screen samples in order to identify compounds or agents (e.g., antibodies or ADCs) that alter a function or activity of tumor cells by interacting with a determinant. In one embodiment, tumor cells are put in contact with an antibody or ADC and the antibody or ADC can be used to screen the tumor for cells expressing a certain target (e.g. RNF43) in order to identify such cells for purposes, including but not limited to, diagnostic purposes, to monitor such cells to determine treatment efficacy or to enrich a cell population for such target-expressing cells.

In yet another embodiment, a method includes contacting, directly or indirectly, tumor cells with a test agent or compound and determining if the test agent or compound modulates an activity or function of the determinant-associated tumor cells for example, changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Screening methods include high throughput screening, which can include arrays of cells (e.g., microarrays) positioned or placed, optionally at pre-determined locations, for example, on a culture dish, tube, flask, roller bottle or plate. High-throughput robotic or manual handling methods can probe chemical interactions and determine levels of expression of many genes in a short period of time. Techniques have been developed that utilize molecular signals, for example via fluorophores or microarrays (Mocellin and Rossi, 2007, PMID: 17265713) and automated analyses that process information at a very rapid rate (see, e.g., Pinhasov et al., 2004, PMID: 15032660). Libraries that can be screened include, for example, small molecule libraries, phage display libraries, fully human antibody yeast display libraries (Adimab), siRNA libraries, and adenoviral transfection vectors.

### VII. PHARMACEUTICAL PREPARATIONS AND THERAPEUTIC USES

1. Formulations and Routes of Administration

The antibodies or ADCs of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers comprise substances that are relatively inert and can facilitate administration of the antibody or can aid processing of the active compounds into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Antibodies for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include Sodium Chloride Injection, Ringer's Solution, or Lactated Ringer's Injection.

Compatible formulations for parenteral administration (e.g., intravenous injection) may comprise ADC or antibody concentrations of from about 10 μg/mL to about 100 mg/mL. In certain selected embodiments antibody or ADC concentrations will comprise 20 μg/mL, 40 μg/mL, 60 μg/mL, 80 μg/mL, 100 μg/mL, 200 μg/mL, 300, μg/mL, 400 μg/mL, 500 μg/mL, 600 μg/mL, 700 μg/mL, 800 μg/mL, 900 μg/mL or 1 mg/mL. In other preferred embodiments ADC concentrations will comprise 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

The compounds and compositions of the invention may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

2. Dosages

The particular dosage regimen, i.e., dose, timing and repetition, will depend on the particular individual, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSA for prostate cancer) or an antigen identified according to the methods described herein; reduction in the number of proliferative or tumorigenic cells, maintenance of the reduction of such neoplastic cells; reduction of the proliferation of neoplastic cells; or delay in the development of metastasis.

The RNF43 antibodies or ADCs of the invention may be administered in various ranges.

These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the RNF43 antibodies or ADCs will be administered (preferably intravenously) at approximately 10, 20, 30, 40, 50, 60, 70, 80, 90 or 100 μg/kg body weight per dose. Other embodiments may comprise the administration of ADCs at about 200, 300, 400, 500, 600, 700, 800, 900, 1000, 1100, 1200, 1300, 1400, 1500, 1600, 1700, 1800, 1900 or 2000 μg/kg body weight per dose. In other preferred embodiments the disclosed conjugates will be administered at 2.5, 3, 3.5, 4, 4.5, 5, 5.5, 6, 6.5, 7, 7.58, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for various RNF43 antibodies or ADCs based on preclinical animal studies, clinical observations and standard medical and biochemical techniques and measurements.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

Anti-RNF43 antibodies or ADCs may be administered on a specific schedule. Generally, an effective dose of the RNF43 conjugate is administered to a subject one or more times. More particularly, an effective dose of the ADC is administered to the subject once a month, more than once a month, or less than once a month. In certain embodiments, the effective dose of the RNF43 antibody or ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed antibodies or ADCs.

In certain preferred embodiments the course of treatment involving conjugated antibodies will comprise multiple doses of the selected drug product over a period of weeks or months. More specifically, antibodies or ADCs of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

3. Combination Therapies

Combination therapies may be useful in preventing or treating cancer and in preventing metastasis or recurrence of cancer. “Combination therapy”, as used herein, means the administration of a combination comprising at least one anti-RNF43 antibody or ADC and at least one therapeutic moiety (e.g., anti-cancer agent) wherein the combination preferably has therapeutic synergy or improves the measurable therapeutic effects in the treatment of cancer over (i) the anti-RNF43 antibody or ADC used alone, or (ii) the therapeutic moiety used alone, or (iii) the use of the therapeutic moiety in combination with another therapeutic moiety without the addition of an anti-RNF43 antibody or ADC. The term “therapeutic synergy”, as used herein, means the combination of an anti-RNF43 antibody or ADC and one or more therapeutic moiety(ies) having a therapeutic effect greater than the additive effect of the combination of the anti-RNF43 antibody or ADC and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the anti-RNF43 antibodies or ADCs described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable.)

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or anti-RNF43 antibody or ADC, or the sum of the therapeutic effects elicited by the anti-RNF43 antibody or ADC or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or anti-RNF43 antibody or ADC, or the sum of the therapeutic effects elicited by the anti-RNF43 antibody or ADC or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the anti-RNF43 antibody or ADC and therapeutic moiety(ies) may be administered to the subject simultaneously, either in a single composition, or as two or more distinct compositions using the same or different administration routes. Alternatively, treatment with the anti-RNF43 antibody or ADC may precede or follow the therapeutic moiety treatment by, e.g., intervals ranging from minutes to weeks. In one embodiment, both the therapeutic moiety and the antibody or ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The antibody and therapeutic moiety(ies) may be administered on alternate days or weeks; or a sequence of anti-RNF43 antibody or ADC treatments may be given, followed by one or more treatments with the additional therapeutic moiety. In one embodiment an anti-RNF43 antibody or ADC is administered in combination with one or more therapeutic moiety(ies) for short treatment cycles. In other embodiments the combination treatment is administered for long treatment cycles. The combination therapy can be administered via any route.

The invention also provides for the combination of anti-RNF43 antibodies or ADCs with radiotherapy. The term “radiotherapy”, as used herein, means, any mechanism for inducing DNA damage locally within tumor cells such as gamma-irradiation, X-rays, UV-irradiation, microwaves, electronic emissions and the like. Combination therapy using the directed delivery of radioisotopes to tumor cells is also contemplated, and may be used in combination or as a conjugate of the anti-RNF43 antibodies disclosed herein. Typically, radiation therapy is administered in pulses over a period of time from about 1 to about 2 weeks. Optionally, the radiation therapy may be administered as a single dose or as multiple, sequential doses.

In other embodiments an anti-RNF43 antibody or ADC may be used in combination with one or more of the chemotherapeutic agents described below.

4. Anti-Cancer Agents

The term “anti-cancer agent” or “chemotherapeutic agent” as used herein is one subset of “therapeutic moieties”, which in turn is a subset of the agents described as “pharmaceutically active moieties”. More particularly “anti-cancer agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. It will be appreciated that in selected embodiments as discussed above, such anti-cancer agents may comprise conjugates and may be associated with antibodies prior to administration. In certain embodiments the disclosed anti-cancer agent will be linked to an antibody to provide an ADC as disclosed herein.

The term “cytotoxic agent”, which can also be an anticancer agent means a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

An anti-cancer agent can include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. Such agents are often administered, and are often most effective, in combination, e.g., in the formulation CHOP. Again, in selected embodiments such anti-cancer agents may be conjugated to the disclosed antibodies.

Examples of anti-cancer agents that may be used in combination with (or conjugated to) the antibodies of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Additional anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

“Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the antibodies or ADCs of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lambrolizumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MDX-1105 and MEDI4736 and combinations thereof.

Other particularly preferred embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

5. Radiotherapy

The present invention also provides for the combination of antibodies or ADCs with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies or ADCs may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VIII. INDICATIONS

The invention provides for the use of antibodies and ADCs of the invention for the diagnosis, theragnosis, treatment and/or prophylaxis of various disorders including neoplastic, inflammatory, angiogenic and immunologic disorders and disorders caused by pathogens. Particularly, key targets for treatment are neoplastic conditions comprising solid tumors, although hematologic malignancies are within the scope of the invention. In certain embodiments the antibodies of the invention will be used to treat tumors or tumorigenic cells expressing a particular determinant (e.g. RNF43). Preferably the “subject” or “patient” to be treated will be human although, as used herein, the terms are expressly held to comprise any mammalian species.

Neoplastic conditions subject to treatment in accordance with the instant invention may be benign or malignant; solid tumors or other blood neoplasia; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In other preferred embodiments, the disclosed antibodies and ADCs are especially effective at treating lung cancer, including the following subtypes: small cell lung cancer and non-small cell lung cancer (e.g. squamous cell non-small cell lung cancer or squamous cell small cell lung cancer). In selected embodiments the antibodies and ADCs can be administered to patients exhibiting limited stage disease or extensive stage disease. In other preferred embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel).

In other particularly preferred embodiments the ADCs of the instant invention may be used to treat colorectal cancer. As used herein, the term “colorectal cancer” is meant to include the well-accepted medical definition that defines colorectal cancer as a medical condition characterized by cancer of cells of the intestinal tract below the small intestine (i.e. the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum). The definition of colorectal cancer used herein is more expansive than the common medical definition but is provided as such since the cells of the duodenum and small intestine may also be amenable to the methods of the present invention. Moreover the compounds of the invention may be used to treat stage I colorectal cancer, stage II colorectal cancer, stage III colorectal cancer or stage IV colorectal cancer.

The invention also provides for a preventative or prophylactic treatment of subjects who present with benign or precancerous tumors. No particular type of tumor or proliferative disorder is excluded from treatment using the antibodies of the invention.

### IX. ARTICLES OF MANUFACTURE

The invention includes pharmaceutical packs and kits comprising one or more containers, wherein a container can comprise one or more doses of an antibody or ADC of the invention. In certain embodiments, the pack or kit contains a unit dosage, meaning a predetermined amount of a composition comprising, for example, an antibody or ADC of the invention, with or without one or more additional agents and optionally, one or more anti-cancer agents.

The kit of the invention will generally contain in a suitable container a pharmaceutically acceptable formulation of the antibody or ADC of the invention and, optionally, one or more anti-cancer agents in the same or different containers. The kits may also contain other pharmaceutically acceptable formulations or devices, either for diagnosis or combination therapy. Examples of diagnostic devices or instruments include those that can be used to detect, monitor, quantify or profile cells or markers associated with proliferative disorders (for a full list of such markers, see above). In particularly preferred embodiments the devices may be used to detect, monitor and/or quantify circulating tumor cells either in vivo or in vitro (see, for example, WO 2012/0128801). In still other preferred embodiments the circulating tumor cells may comprise tumorigenic cells. The kits contemplated by the invention can also contain appropriate reagents to combine the antibody or ADC of the invention with an anti-cancer agent or diagnostic agent (e.g., see U.S. Pat. No. 7,422,739).

When the components of the kit are provided in one or more liquid solutions, the liquid solution can be non-aqueous, however, an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the antibody or ADC of the invention in combination with additional therapeutics or agents, the solution may be pre-mixed, either in a molar equivalent combination, or with one component in excess of the other. Alternatively, the antibody or ADC of the invention and any optional anti-cancer agent or other agent can be maintained separately within distinct containers prior to administration to a patient.

The kit can comprise one or multiple containers and a label or package insert in, on or associated with the container(s), indicating that the enclosed composition is used for diagnosing or treating the disease condition of choice. Suitable containers include, for example, bottles, vials, syringes, etc. The containers can be formed from a variety of materials such as glass or plastic. The container(s) can comprise a sterile access port, for example, the container may be an intravenous solution bag or a vial having a stopper that can be pierced by a hypodermic injection needle.

In some embodiments the kit can contain a means by which to administer the antibody and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### X. MISCELLANEOUS

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XI. REFERENCES

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### XII. XV. SEQUENCE LISTING SUMMARY

Appended to the instant application is a sequence listing comprising a number of nucleic acid and amino acid sequences. The following Table 3 provides a summary of the included sequences.

TABLE 3

| SEQ ID NO. | Description |
|---|---|
| 1 | Kappa light chain constant region protein |
| 2 | IgG1 heavy chain constant region protein |
| 3 | Amino acid sequence of the ECD of RNF43 |
| 4 | Amino acid sequence of the ECD of ZNRF3 |
| 5 | Amino acid sequence of full length RNF43 |
| 6 | Amino acid sequence of full length ZNRF3 |
| 7-20 | Save |
| 21 | SC37.1 VL DNA |
| 22 | SC37.1 VL protein |
| 23 | SC37.1 VH DNA |
| 24 | SC37.1 VH protein |
| 25-252 | Additional murine clones |
| 253-270 | Humanized clones |
| 271-281 | Full length protein sequences of humanized clones |
| 282, 283, 284 | hSC37.2 CDRL1, CDRL2, CDRL3 |
| 285, 286, 287 | hSC37.2 CDRH1, CDRH2, CDRH3 |
| 288, 289, 290 | hSC37.17 CDRL1, CDRL2, CDRL3 |
| 291, 292, 293 | hSC37.17 CDRH1, CDRH2, CDRH3 |
| 294, 295, 296 | hSC37.39 CDRL1, CDRL2, CDRL3 |
| 297, 298, 299 | hSC37.39 CDRH1, CDRH2, CDRH3 |
| 300, 301, 302 | hSC37.67 CDRL1, CDRL2, CDRL3 |
| 303, 304, 305 | hSC37.67 CDRH1, CDRH2, CDRH3 |
| 306 | hSC37.67v1 CDRL3 |

Note that hSC37.67v1, a variant which is derived from the humanized antibody hSC37.67, only differs from hSC37.67 by a single amino acid in CDRL3 of its light chain variable region. The heavy chains of hSC37.67 and hSC37.67v1 are identical. Example 10 below describes the generation of these antibodies in more detail.

### XIII. EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 4 as follows:

TABLE 4

| Tumor Type | Abbreviation | Tumor subtype | Abbreviation |
|---|---|---|---|
| Breast | BR | | |
| | | estrogen receptor positive and/or | BR-ERPR |
| | | progesterone receptor positive | |
| | | ERBB2/Neu positive | BR-ERBB2/Neu |
| | | HER2 positive | BR-HER2 |
| | | triple-negative | TNBC |
| | | luminal A | BR-lumA |
| | | claudin subtype of triple-negative | TNBC-CL |
| Colorectal | CR | | |
| endometrial | EM | | |
| Gastric | GA | | |
| | | diffuse adenocarcinoma | GA-Ad-Dif/Muc |
| | | intestinal adenocarcinoma | GA-Ad-Int |
| | | stromal tumors | GA-GIST |
| glioblastoma | GB | | |
| head and neck | HN | | |
| Kidney | KDY | | |
| | | clear renal cell carcinoma | KDY-CC |
| | | papillary renal cell carcinoma | KDY-PAP |
| | | transitional cell or urothelial | KDY-URO |
| | | carcinoma | |
| | | unknown | KDY-UNK |
| Liver | LIV | | |
| | | hepatocellular carcinoma | LIV-HCC |
| | | cholangiocarcinoma | LIV-CHOL |
| Lymphoma | LN | | |
| Lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| Ovarian | OV | | |
| | | clear cell | OV-CC |
| | | endometroid | OV-END |
| | | mixed subtype | OV-MIX |
| | | malignant mixed mesodermal | OV-MMMT |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| Pancreatic | PA | | |
| | | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine type | PA-PACe |
| | | ductal adenocarcinoma | PA-PDAC |
| | | ampullary adenocarcinoma | PA-AAC |
| Prostate | PR | | |
| Skin | SK | | |
| | | melanoma | MEL |
| | | squamous cell carcinomas | SK-SCC |
| | | uveal melanoma | UVM |

### Example 1

### Identification of RNF43 Expression Using Whole Transcriptome Sequencing

To characterize the cellular heterogeneity of solid tumors as they exist in cancer patients and identify clinically relevant therapeutic targets, a large PDX tumor bank was developed and maintained using art recognized techniques. The PDX tumor bank, comprising a large number of discrete tumor cell lines, was propagated in immunocompromised mice through multiple passages of tumor cells originally obtained from cancer patients afflicted by a variety of solid tumor malignancies. Low passage PDX tumors are representative of tumors in their native environments, providing clinically relevant insight into underlying mechanisms driving tumor growth and resistance to current therapies.

Tumor cells can be divided broadly into two types of cell subpopulations: non-tumorigenic cells (NTG) and tumor initiating cells (TICs). TICs have the ability to form tumors when implanted into immunocompromised mice. Cancer stem cells (CSCs) are a subset of tumor initiating cells and are able to self-replicate indefinitely while maintaining the capacity for multilineage differentiation. Tumor progenitor cells (TProgs) are also a subset of TICs, and like CSCs, have the ability to fuel tumor growth in a primary transplant. However, unlike CSCs, they are not able to recapitulate the cellular heterogeneity of the parental tumor and are less efficient at reinitiating tumorigenesis in subsequent transplants because TProgs are typically only capable of a finite number of cell divisions. In order to perform whole transcriptome analysis, PDX tumors from the tumor bank were resected from mice after they reached 800-2,000 mm³. Resected PDX tumors were dissociated into single cell suspensions using art-recognized enzymatic digestion techniques (see, for example, U.S.P.N. 2007/0292414). Dissociated bulk tumor cells were incubated with 4′,6-diamidino-2-phenylindole (DAPI) to detect dead cells, anti-mouse CD45 and H-2K^{d} antibodies to identify mouse cells and anti-human EPCAM antibody to identify human cells. In addition, the tumor cells were incubated with fluorescently-conjugated anti-human CD46 and/or CD324 antibodies, and in some cases CD66c, to identify CD46⁺CD324⁺CSC and CD46⁻CD324⁻ NTG and were then sorted using a FACSAria cell sorter (BD Biosciences) (see U.S.P.N.s 2013/0260385, 2013/0061340 and 2013/0061342). In the case of CR4, the TProg population was identified as CD46⁺/CD324⁺/CD66c⁺ whereas the CSC population was identified as CD46⁺/CD324⁺/CD66c⁻.

RNA was extracted from tumor cells or normal tissue by lysing the cells in RLTplus RNA lysis buffer (Qiagen) supplemented with 1% 2-mercaptoethanol, freezing the lysates at −80° C. and then thawing the lysates for RNA extraction using an RNeasy isolation kit (Qiagen). RNA was quantified using a Nanodrop spectrophotometer (Thermo Scientific) and/or a Bioanalyzer 2100 (Agilent Technologies). The resulting total RNA preparations were assessed by genetic sequencing and gene expression analyses.

Whole transcriptome sequencing of high quality RNA was performed using two different systems. Some samples were analyzed using an Applied Biosystems (ABI) Sequencing by Oligo Ligation/Detection (SOLiD) 4.5 or SOLiD 5500x1 next generation sequencing system (Life Technologies). Other samples were analyzed using Illumina HiSeq 2000 or 2500 next generation sequencing system (Illumina).

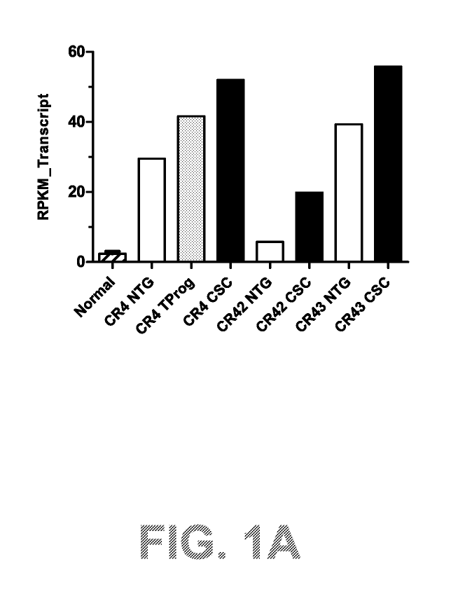

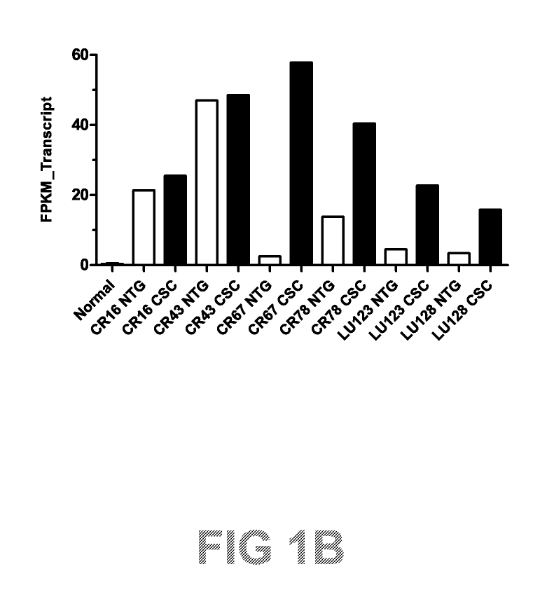

SOLiD whole transcriptome analysis was performed with cDNA, generated from 1 ng RNA from bulk tumor samples using either a modified whole transcriptome protocol from ABI designed for low input total RNA or the Ovation RNA-Seq System V2™ (NuGEN Technologies). The resulting cDNA library was fragmented, and barcode adapters were added to allow pooling of fragment libraries from different samples during sequencing runs. Data generated by the SOLiD platform mapped to 34,609 genes as annotated by RefSeq version 47 using NCBI version hg19.2 of the published human genome and provided verifiable measurements of RNA levels in most samples. Sequencing data from the SOLiD platform is nominally represented as a transcript expression value using the metrics RPM (reads per million) or RPKM (read per kilobase per million) mapped to exon regions of genes, enabling basic gene expression analysis to be normalized and enumerated as RPM_Transcript or RPKM_Transcript. Compared to the average of all normal cells tested, RNF43 mRNA was elevated in the following CR PDX tumor cell lines: CR4, CR42 and CR43 (FIG. 1A). The normal tissues that were tested were colon, heart, liver, lung, kidney, pancreas and ovary. There was also higher expression of RNF43 mRNA in CSCs compared to NTG cells in the same CR PDX lines. In addition the CR4 PDX line comprised a subpopulation of TProg cells that also expressed RNF43 mRNA (FIG. 1A), which was observed to be intermediate to levels observed in CSC and NTG cells.

Illumina whole transcriptome analysis was performed with cDNA that was generated using 5 ng total RNA extracted from CR and LU PDX tumor cells. The tumor cells were sorted for CSC and NTG cell populations and RNA was extracted as described for SOLiD whole transcriptome analysis. The library was created using the TruSeq RNA Sample Preparation Kit v2 (Illumina). The resulting cDNA library was fragmented and barcoded. Sequencing data from the Illumina platform is nominally represented as a fragment expression value using the metrics FPM (fragment per million) or FPKM (fragment per kilobase per million) mapped to exon regions of genes, enabling basic gene expression analysis to be normalized and enumerated as FPM_Transcript or FPKM_Transcript. Compared to the NTG population, expression of RNF43 mRNA was elevated in the CSC tumor cell subpopulation of LU-SCC (LU128), LU-Ad (LU123), and CR PDX tumor cell lines (CR16, CR43, CR67, CR78) (FIG. 1B). RNF43 mRNA expression was also higher in CSCs compared to the relevant normal tissue in the following organs: esophagus, trachea, stomach, spleen, skin, pancreas, lung, liver, kidney, heart and colon (FIG. 1B).

The identification of elevated RNF43 mRNA expression in CR, LU-Ad and LU-SCC tumors indicated that RNF43 merited further evaluation as a potential diagnostic and/or immunotherapeutic target. Furthermore, increased expression of RNF43 in CSC compared to NTG in CR, LU-Ad and LU-SCC tumors indicates that RNF43 is a good marker of tumorigenic cells in these tumor types.

### Example 2

### Expression of RNF43 mRNA in Tumors Using qRT-PCR

To confirm mRNA expression of RNF43 in tumor cells, qRT-PCR was performed on various PDX cell lines using the Fluidigm BioMark™ HD System according to industry standard protocols. RNA was extracted from bulk and sorted PDX tumor cells as described in Example 1. 1 ng of RNA was converted to cDNA using the High Capacity cDNA Archive kit (Life Technologies) according to the manufacturer's instructions. cDNA material, pre-amplified using a RNF43-specific Taqman assay, was then used for subsequent qRT-PCR experiments.

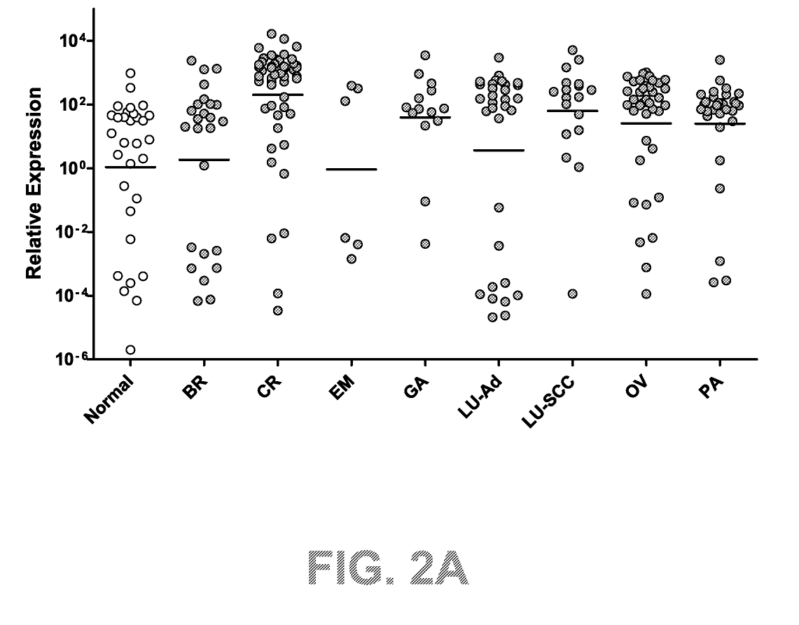

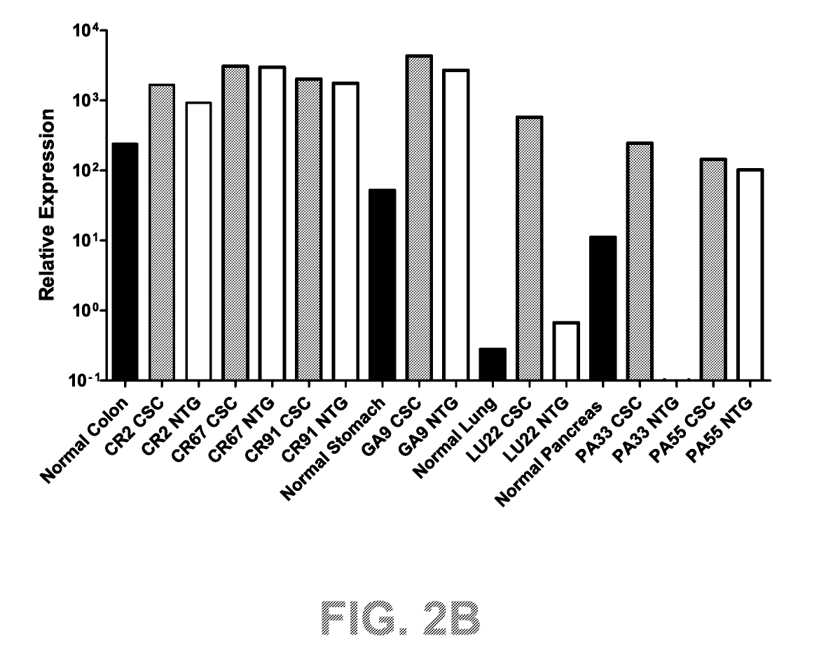

Expression of RNF43 in normal tissues (adipose, brain, melanocytes, PBMC and sorted B, monocytes, NK and T cells, normal bone marrow, salivary gland, testes, thymus, thyroid, adrenal, artery, vein, colon, dorsal root ganglion, esophagus, heart, kidney, liver, lung, pancreas, skeletal muscle, skin, small intestine, spleen, stomach, trachea, and vascular smooth muscle cells) was lower compared to expression in subsets of the following PDX tumor cell lines: BR, CR, EM, GA, LU-Ad, LU-SCC, PA and OV (FIG. 2A). In addition, qRT-PCR analysis was carried out, as described above, on CR, GA, LU and PA PDX tumor cell lines that had been sorted for CSCs and NTG cells as described in Example 1. The CR (CR91, CR67, CR2), GA (GA9), LU-SCC (LU22) and PA (PA33, PASS) PDX lines that were tested showed increased mRNA expression in CSC compared to NTG, confirming the findings in Example 1, namely that RNF43 is a good biomarker of tumorigenic cancer cells (FIG. 2B). In sum, these data demonstrate that RNF43 is expressed in a number of tumors and may be a good target for the development of an antibody-based therapeutic in these indications.

### Example 3

### Determination of Expression of RNF43 mRNA in Tumors Using Microarray

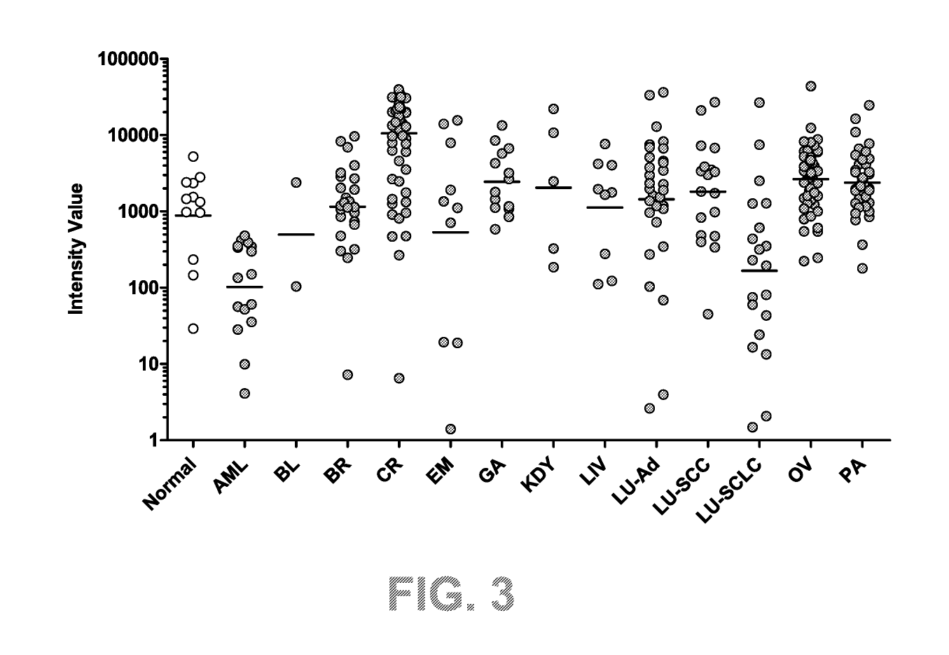

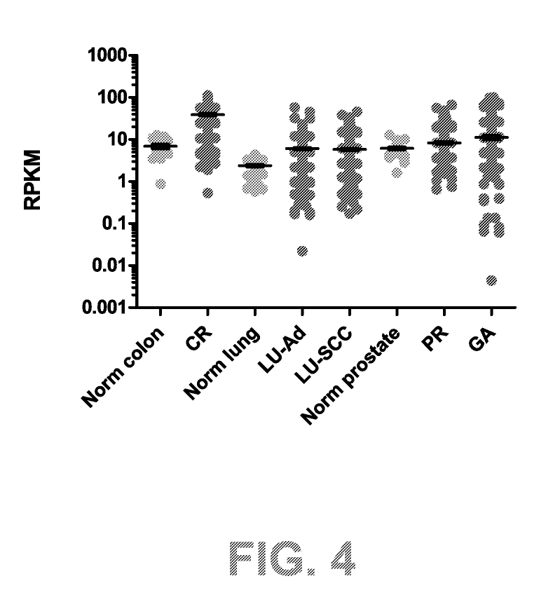

RNF43 expression was determined using microarray analyses to confirm the results obtained through qRT-PCR and whole transcriptome analysis. 1-2 μg of whole tumor total RNA was derived, substantially as described in Example 1, from PDX cell lines comprising a variety of cancer types. The samples were analyzed using the Agilent SurePrint GE Human 8x60 v2 microarray platform, which contains 50,599 biological probes designed against 27,958 genes and 7,419 lncRNAs in the human genome. Standard industry practices were used to normalize and transform the intensity values to quantify gene expression for each sample. The normalized intensity of RNF43 expression in each sample is plotted in FIG. 3 and the geometric mean derived for each tumor type is indicated by the horizontal bar.

FIG. 3 shows that RNF43 mRNA expression is elevated compared to normal tissues (stomach, spleen, skin, PBMC, pancreas, ovary, lung, liver, kidney, heart, colon and breast) in CR, as well as subsets of EM, GA, KDY, LU-Ad, LU-SCC, PA, and OV. The observation of elevated RNF43 expression in a variety of PDX tumor cell lines confirms the results of Examples 1 and 2. Such findings further support the observed association between RNF43 expression levels and tumor cells, particularly in the CR, and subsets of GA, LU-Ad, LU-SCC, OV and PA tumors

### Example 4

### RNF43 Expression in Tumors Using the Cancer Genome Atlas

Overexpression of RNF43 mRNA in various tumors was confirmed using a large, publicly available dataset of primary tumors and normal samples known as The Cancer Genome Atlas (TCGA). RNF43 expression data from the IlluminaHiSeq_RNASeqV2 platform and the IlluminaHiSeq_RNASeq platform was downloaded from the TCGA Data Portal (https://tcga-data.nci.nih.gov/tcga/tcgaDownload.jsp) and parsed to aggregate the reads from the individual exons of each gene to generate a single value read per kilobase of exon per million mapped reads (RPKM). FIG. 4 shows that RNF43 expression is elevated in primary LU-Ad, LU-SCC, CR, GA as well as PR tumors, relative to normal tissues found in the TCGA database. These data confirm the results in Examples 1-3, implying there is a good therapeutic index above normal tissues and therefore anti-RNF43 antibodies and ADCs may be useful therapeutics for the treatment of these tumors.

### Example 5

### Cloning and Expression of Recombinant RNF43 Proteins and Engineering of Cell Lines Overexpressing Cell Surface RNF43 Proteins

DNA Fragments Encoding Human RNF43 Proteins.

To generate all cellular materials pertaining to the human RNF43 (hRNF43) protein (GenBank accession NP_060233), a cDNA clone encoding the full length hRNF43 open reading frame was purchased (RC214013; Origene). This cDNA clone was used for all subsequent engineering of constructs expressing the mature hRNF43 protein or fragments thereof.

To create a lentiviral vector plasmid encoding the hRNF43 protein, the hRNF43 open reading frame was amplified from the above template using PCR, and the resultant PCR product was subcloned into the multiple cloning site (MCS) of a lentiviral expression vector pCDH-EF1-MCS-T2A-GFP (System Biosciences), which had been previously modified to introduce nucleotide sequences encoding an I_{kκ} signal peptide followed by a DDDK epitope tag upstream of the MCS. The T2A sequence downstream of the MCS promotes ribosomal skipping of a peptide bond condensation, resulting in expression of two independent proteins: high level expression of DDDK-tagged cell surface proteins encoded upstream of the T2A peptide, with co-expression of the GFP marker protein encoded downstream of the T2A peptide. This cloning step yielded the lentiviral vector plasmid pL120-hRNF43-NFlag.

DNA Fragments Encoding Rat and Cynomolgus RNF43 Proteins.

To generate all molecular and cellular materials required in the present invention pertaining to the rat RNF43 protein (rRNF43), synthetic cDNA clone encoding a protein identical to the rat RefSeq RNF43 protein (GenBank accession NP_001129393) was designed and purchased from GeneWiz. To generate all molecular and cellular materials required in the present invention pertaining to the cynomolgus monkey (Macaca fascicularis) RNF43 protein (cRNF43), the cRNF43 open reading frame sequence was first deduced by BLASTing the DNA sequence encoding the hRNF43 protein versus the cynomolgus whole genome shotgun contigs sequence database at the NCBI, observing that exon/intron boundaries were conserved between the human and cynomolgus genes, and assembling a putative cynomolgus open reading frame encoding cRNF43. Analysis of the results indicated that the hRNF43 and cRNF43 proteins were 96.2% identical. A synthetic DNA clone encoding this predicted cRNF43 protein was designed and purchased from GeneWiz.

The rat and cynomolgus DNA clones were used as templates for various PCR reactions to generate chimeric fusion genes for either the rRNF43 or cRNF43 ECD and a Histidine tag or human IgG2 Fc tag. Briefly, the DNA encoding the predicted ECD domains for rRNF43 or cRNF43, as deduced by database annotation or by sequence alignment with the hRNF43 protein, were amplified by PCR. These PCR products were subcloned into a CMV driven expression vector in-frame and downstream of an I_{gκ} signal peptide sequence and upstream of either a Histidine tag or a human IgG2 Fc cDNA, using standard molecular techniques. These CMV-driven expression vectors permit high level transient expression in HEK293T cells. Suspension or adherent cultures of HEK293T cells were transfected with these expression constructs, using polyethylenimine polymer as the transfecting reagent. Three to five days after transfection, the recombinant His-tagged or Fc-tagged proteins were purified from clarified cell-supernatants using an AKTA explorer and either Nickel-EDTA (Qiagen) or MabSelect SuRe™ Protein A (GE Healthcare Life Sciences) columns, respectively.

Cell Line Engineering

Engineered cell lines overexpressing the hRNF43 protein were constructed using the pL120-hRNF43-NFlag lentiviral vector, described above, to transduce HEK293T cell lines using standard lentiviral transduction techniques well known to those skilled in the art. hRNF43-expressing cells were selected using FACS of high-expressing HEK293T subclones (e.g., cells that were strongly positive for both GFP and the DDDK epitope tag).

### Example 6

### Generation of Anti-RNF43 Antibodies

Anti-RNF43 murine antibodies were produced in two different immunizations as follows. In the first immunization, one female Balb/c mouse was inoculated via footpad with 10 μg of recombinant human RNF43-Fc protein (rhRNF43-Fc, R&D Systems; #7964-RN) emulsified in TiterMax® and CpG adjuvant. Following the initial inoculation the mouse was injected seven times (twice per week) with 5 μg rhRNF43-Fc protein emulsified with Alum, PBS and CpG. The final inoculation comprised 5 μg rhRNF43-Fc protein in PBS. In the second immunization, six mice (two each of the following strains: BALB/c, CD-1, FVB) were immunized with 10 μg hRNF43-His protein (Sino) twice per week for 4 weeks followed by a final inoculation two weeks later.

The mice were sacrificed and draining lymph nodes (popliteal, inguinal, and medial iliac) were dissected and used as a source for antibody producing cells. A single cell suspension of B cells (60×10⁶ cells) were fused with non-secreting P3x63Ag8.653 myeloma cells (ATCC # CRL-1580) at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM condimed, 1 mM nonessential amino acids, 1 mM HEPES, 100 IU penicillin-streptomycin, and 50 μM 2-mercaptoethanol, and were cultured in a T225 flask in 100 mL selection medium. The flask was placed in a humidified 37° C. incubator containing 5% CO₂ and 95% air for 6 days.

On day 6 after the fusion the hybridoma library cells were collected from the flask and the library was stored in liquid nitrogen. Frozen vials were thawed into T75 flasks and on the following day the hybridoma cells were plated at one cell per well (using the FACSAria I cell sorter) in 90 μL of supplemented hybridoma selection medium (as described above) into 12 Falcon 384-well plates.

The hybridomas were cultured for 10 days and the supernatants were screened for antibodies specific to hRNF43 using flow cytometry performed as follows. 1×10⁵ per well of HEK293T cells stably transduced with hRNF43 were incubated for 30 mins. with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS for 15 mins. Cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

### Example 7

### Characteristics of Anti-RNF43 Antibodies

Anti-RNF43 antibodies generated in Example 6 were characterized in terms of (i) isotype, (ii) affinity for RNF43; and (iii) cross reactivity with ZNRF3. In addition, the antibodies were grouped in bins on the basis of whether they competed with each other for binding to human RNF43 protein.

The isotype of a representative number of antibodies was determined using the Milliplex mouse immunoglobulin isotyping kit (Millipore) according to the manufacturer's protocols. Those antibodies for which no clear signal could be obtained were assigned an isotype based on sequence analysis following protocols standard in the art. Results of the isotyping analyses for the unique RNF43-specific antibodies can be seen in FIG. 5A.

The affinity of select antibodies for hRNF43 protein was determined using surface plasmon resonance using a BIAcore 2000 (GE Healthcare) machine. An anti-mouse antibody capture kit was used to immobilize mouse anti-RNF43 antibodies on a CM5 biosensor chip. Prior to each antigen injection cycle, murine antibodies at a concentration of 0.05-1 μg/mL were captured on the surface with a contact time of 1 min. and a flow rate of 5 μL/min. The captured antibody loading from baseline was 80-140 response units. Following antibody capture and 1 min. baseline, monomeric hRNF43-His antigen generated in Example 5 was flowed over the surface at concentrations ranging from 10-200 nM for a 1.5 min. association phase followed by a 5 min. dissociation phase at a flow rate of 10 μL/min. A similar protocol was used for measuring binding affinity of humanized antibodies except that an anti-human antibody capture kit was used. The data was processed by subtracting a control non-binding antibody surface response from the specific antibody surface response and data was truncated to the association and dissociation phase. The resulting response curves were used to fit a 1:1 Langmuir binding model and to generate an apparent affinity using the calculated kₒₙ and k_{off} kinetics constants using BiaEvaluation Software 3.1 (GE Healthcare). The selected antibodies exhibited affinities for hRNF43 in the nanomolar range (FIG. 5A).

An ELISA assay using the Meso Scale Discovery platform was performed to test the ability of selected anti-RNF43 antibodies generated in Example 6 to bind ZNRF3, a functional homolog of RNF43. While ZNRF3 is functionally homologous to RNF43, there is only 20% sequence homology overall (FIG. 5B), thus cross reactivity of the anti-RNF43 antibodies with ZNRF3 was not expected. Plates were coated with human ZNRF3 or RNF43 (both, R&D Systems) at 0.5 μg/mL in PBS and incubated overnight at 4° C. After the plates were washed with PBS, 0.05% tween20 (PBST), they were blocked with 35 μl of 3% (w/v) BSA in PBS for 60 mins. at room temperature. The plates were washed in PBST and 10 μl of titrated anti-RNF43 antibodies (500 ng/ml-0.032 ng/ml) diluted in PST, 0.05% tween, 1% BSA(w/v) (PBSTA) was added to the plates and incubated for 60 mins. After washing with PBST, 10 μL/well sulfo tag-labeled goat anti-mouse IgG (Meso Scale Discovery, # R32AC-5) at 0.5 μg/ml in PBSTA was added for 30 mins. at room temperature. MSD SULFO-TAG NHS-Ester is an amine reactive, N-hydroxysuccinimide ester which readily couples to primary amine groups of proteins under mildly basic conditions to form a stable amide bond. Plates were washed in PBST and MSD Read Buffer T with surfactant was diluted to 1× in water and 35 μL was added to each well. Plates were read on an MSD Sector Imager 2400. All of the antibodies that were tested (e.g. SC37.2, SC37.4, SC37.8, SC37.10, SC37.17, SC37.28, SC37.39, SC37.226 and SC37.236) bound RNF43 but displayed no binding to ZNRF3. In addition, the negative control, mouse IgG1, did not bind either protein. In contrast, RNF43 or ZNRF3 protein that was fused to a human FC or fused to a murine anti-human FC antibody, exhibited binding to both proteins (positive control).

Antibodies were grouped into bins using a multiplexed competition immunoassay (Luminex). 100 μl of each unique anti-RNF43 antibody (capture mAb) at a concentration of 10 μg/mL was incubated for 1 hour with magnetic beads (Luminex) that had been conjugated to an anti-mouse kappa antibody (Miller et al., 2011, PMID: 21223970). The capture mAb/conjugated bead complexes were washed with PBSTA buffer (1% BSA in PBS with 0.05% Tween20) and then pooled. Following removal of residual wash buffer the beads were incubated for 1 hour with 2 μg/mL hRNF43-His protein, washed and then resuspended in PBSTA. The pooled bead mixture was distributed into a 96 well plate, each well containing a unique anti-RNF43 antibody (detector mAb) and incubated for 1 hour with shaking. Following a wash step, anti-mouse kappa antibody (the same as that used above), conjugated to PE, was added at a concentration of 5 μg/ml to the wells and incubated for 1 hour. Beads were washed again and resuspended in PBSTA. Mean fluorescence intensity (MFI) values were measured with a Luminex MAGPIX instrument. Antibody pairing was visualized as a dendogram of a distance matrix computed from the Pearson correlation coefficients of the antibody pairs. Binning was determined on the basis of the dendogram and analysis of the MFI values of antibody pairs. The results shown in FIG. 5A demonstrate that the exemplary antibodies that were screened can be grouped into at least six unique bins (A-F), wherein the members of each bin compete with each other for binding to hRNF43 protein.

### Example 8

### Effect of Anti-RNF43 Antibodies on WNT Signaling

The WNT pathway is a critical developmental and stem cell-associated signaling pathway regulating cell growth and differentiation. In the canonical WNT/β-catenin signaling pathway (FIG. 6A), WNT ligands bind a complex of a Frizzled (FZD) receptor and a LRP5/6 co-receptor, initiating the signaling cascade resulting in the inhibition of the protein GSK3, one result of which is the stabilization of the normally labile β-catenin protein found in the cytoplasm. Stabilized β-catenin is then able to accumulate, enter the nucleus, and form complexes with TCF/LEF transcription factors to activate genes containing binding sites for these transcriptional activators. The canonical WNT pathway is regulated extensively at the receptor-ligand level, with multiple activating and inhibitory feedback loops comprised of various soluble decoy receptors (e.g., SFRPs and FRZB), factors that bind WNT itself or modulate its bioactivity (e.g., WIF and NOTUM), or factors that modulate FZD receptor turnover (e.g., RNF43, ZNRF3), and still more elaborate loops comprised of proteins that modulate the modulators (e.g., LGRs and RSPOs). Together these agonist, antagonist and anti-antagonist networks enable a fine control over the strength and duration of a powerful and pleiotropic signaling pathway. Specifically relevant to the present invention are two antagonist and anti-antagonist interactions: the first, an antagonistic interaction in which RNF43, by means of its ability to promote the endocytosis of FZD receptors, down-modulates the WNT-mediated activation of genes containing TCF/LEF binding sites i.e. decreases WNT signaling; and the second, an anti-antagonistic interaction in which the interaction of R-spondins with RNF43 leads to the membrane clearance of RNF43, which promotes increased FZD residence at the cell surface, thereby up-modulating or increasing WNT signaling.

An ELISA assay was used to test the ability of the anti-RNF43 antibodies generated in Example 6 to block binding of RNF43 to human R-spondin (RSPO). Antibodies that functionally block R-spondin interactions with RNF43 are denoted as being in Group II in FIG. 6A. Plates were coated with human RSPO3 (R&D Systems, #3500 RS/CF), which is a representative member of the RSPO protein family, at 0.25 μg/mL in PBS and incubated overnight at 4° C. After the plates were washed with PBS, 0.05% tween20 (PBST), they were blocked with 3% (w/v) BSA in PBS for 90 mins. at 37° C. During the blocking process, 5 ng/ml rhRNF43Fc (R&D Systems; #7964-RN) was incubated with or without 10 μg/mL anti-RNF43 antibody for 60 mins. in 1% (w/v) BSA in PBS+0.05% tween 20 (PBSA). The plates were washed in PBST and 100 μl of the antibody/protein mixture was added to the plates and incubated for 90 mins. After washing with PBST, 50 μL/well HRP-labeled goat anti-human IgG diluted 1:2,000 in PBSA was added for 1 hour at room temperature. The plates were washed and developed by the addition of 100 μL/well of the TMB substrate solution (Thermo Scientific) for 5 mins. at room temperature. An equal volume of 1 M H₂SO₄ was added to stop substrate development. The samples were then analyzed by spectrophotometer at OD 450. The results for exemplary RNF43-specific antibodies can be seen in tabular form in FIG. 5A. It can be seen that a wide range of blocking activities can be observed by the antibodies in this assay.

To determine whether the anti-RNF43 antibodies of the invention modulate the canonical WNT signaling pathway, a stable population of cells containing a reporter for the activation of the canonical WNT signaling pathway were used in various studies of antibody function. These cells, termed 293.TCF cells, were generated by transducing HEK293T cells with a lentiviral vector, pGreenFire1-TCF (System Biosciences), which encodes a bifunctional GFP and luciferase reporter cassette under the control of a minimal CMV reporter linked to four tandem repeats of the transcriptional response elements for the TCF family of transcription factors (e.g., WREs). Thus, activation of the canonical WNT signaling pathway in these cells will result in the stabilization of β-catenin in complexes with the TCF/LEF transcription factors, leading to the activation of the luciferase reporter gene with consequent production of luminescence upon addition of appropriate luciferase substrate and cofactors. The 293.TCF cells were used in a WNT3A canonical signaling assay as follows: 2.5×10⁴ 293. TCF cells were plated per well of a 96-well tissue culture plate in 50 μL of serum-free DMEM medium. After 24 hours of serum starvation, 25 μL of various dilutions of conditioned medium (CM) from L/WNT3A cells (ATCC CRL-2647; Willert, 2003) or undiluted CM from parental L-cells (ATCC CRL-2648) along with 25 μL of DMEM+0.2% FBS were added to each well. Eighteen hours after addition of CM, 100 μL of One-Glo solution (ProMega Corp) was added to each well. The contents of each well were then mixed thoroughly to lyse the cells, 100 μL of lysate was transferred to black 96-well plates, and the luminescence in each well was read after 5 mins. using a Wallac Victor3 Multilabel Counter (Perkin-Elmer Corp). The cells exposed to differing concentrations of CM containing WNT3A typically showed between 2 and 6-fold induction of luciferase signal relative to cells exposed to L-cell control CM (Representative data shown in FIG. 6B). Furthermore, the 293.TCF cells responded as expected following (1) dilution of WNT3A+CM media from 25% to 3%, which resulted in a decrease in WNT reporter activation and a decrease in luminescence, or (2) treatment with 20 mm LiCl, a chemical known to very efficiently inhibit GSK3 and therefore activate canonical WNT signaling response genes, which was indicated by a 12-fold increase in luminescence over treatment with L-cell control CM (data not shown).

The 293.TCF cells were further modified to create 293.TCF.37 lines by transduction of 293.TCF cells using the pL120-hRNF43-NFlag lentiviral vector, described in Example 5 above. The 293.TCF.37 cell lines overexpress RNF43. Bulk populations of 293.TCF.37 cells were then treated with WNT3A+CM or control CM. As expected for the biological function of RNF43, WNT3A-activated luciferase reporter expression was blocked relative to the parental 293.TCF cells (FIG. 6B). Treatment of 293.TCF.37 cells with 20 mM LiCl was able to stimulate luciferase reporter expression above that observed for control CM (data not shown).

The ability of various anti-RNF43 antibodies to modulate WNT signaling activity was determined as follows. 2.5×10⁴ 293. TCF.37 cells, in 50 μL of serum-free DMEM medium, were plated in each well of a 96-well tissue culture plate. After 24 hours of serum starvation, 5 μg/ml of anti-RNF43 antibody in WNT3A+CM were added to the cells. FIG. 5A shows in tabular form, the fold increase in the luciferase reporter activity following antibody treatment relative to WNT3A+CM alone. Some antibodies of the invention resulted in an elevation of the WNT3A-mediated luciferase signal (e.g. SC37.231, which resulted in a 3-fold increase in the luciferase signal) whereas others reduced the WNT3A-mediated luciferase signal (e.g., SC37.77, which resulted in about a 2-fold decrease in the luciferase signal), and still other antibodies did not change the WNT3A-mediated luciferase signal (e.g., SC37.170). Together, these data indicate a variety of anti-RNF43 antibodies were obtained with differing functional effects.

### Example 9

### Sequencing of Anti-RNF43 Antibodies

Anti-RNF43 antibodies were generated as described above and then sequenced. Total RNA was purified from selected hybridoma cells using the RNeasy Miniprep Kit (Qiagen) according to the manufacturer's instructions. Between 10⁴ and 10⁵ cells were used per sample. The quality of the RNA preparations was determined by fractionating 3 μL in a 1% agarose gel before being stored at −80° C. until used.

The variable region of the Ig heavy chain of each hybridoma was amplified using a 5′ primer mix comprising thirty two mouse specific leader sequence primers designed to target the complete mouse VH repertoire in combination with a 3′ mouse Cγ primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of eight RT-PCR reactions were run for each hybridoma, four for the Vκ light chain and four for the Vγ heavy chain. For antibodies containing a lambda light chain, amplification was performed using three 5′ primers designed to prime on the V_{λ} leader sequences in combination with one reverse primer specific to the mouse lambda constant region. PCR reaction mixtures included 3 μL of RNA, 0.5 μL of 100 μM of either heavy chain or light chain primers (custom synthesized by IDT), 5 μL of 5×RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 50° C. for 30 mins., 95° C. for 15 mins. followed by 30 cycles of (95° C. for 30 seconds, 48° C. for 30 seconds, 72° C. for 1 min). There was then a final incubation at 72° C. for 10 mins.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands. Nucleotide sequences were analyzed using the IMGT sequence analysis tool (http://www.imgt.org/IMGTmedical/sequence_analysis.html) to identify germline V, D and J gene members with the highest sequence homology. The derived sequences were compared to known germline DNA sequences of the Ig V- and J-regions by alignment of VH and VL genes to the mouse germline database using a proprietary antibody sequence database.

FIG. 7A depicts the contiguous amino acid sequences of numerous novel murine light chain variable regions from anti-RNF43 antibodies and exemplary humanized light chain variable regions derived from the variable light chains of representative murine anti-RNF43 antibodies. FIG. 7B depicts the contiguous amino acid sequences of novel murine heavy chain variable regions from the same anti-RNF43 antibodies and humanized heavy chain variable regions derived from the same murine antibodies providing the humanized light chains. Unique murine light and heavy chain variable region amino acid sequences are provided in SEQ ID NOS: 22-252 even, numbers while humanized light and heavy chain variable region amino acid sequences are provided in SEQ ID NOS: 254-270, even numbers.

Taken together FIGS. 7A and 7B provide the annotated sequences of numerous unique murine anti-RNF43 antibodies. However a number of duplicate antibodies were generated, having the same variable region light chain and variable region heavy chain as the unique antibodies listed in FIGS. 7A and 7B and are listed in parenthesis after the relevant unique antibody. The antibodies were termed: SC37.1, SC37.2, SC37.3, SC37.4, SC37.6, SC37.7, SC37.8, SC37.9 (identical to SC37.59 and SC37.69), SC37.10, SC37.11, SC37.12, SC37.13, SC37.15, SC37.16, SC37.17, SC37.19 (identical to SC37.33, SC37.35, SC37.52, SC37.55, SC37.58 and SC37.71), SC37.20 (identical to SC37.30, SC37.34, SC37.36, SC37.38, SC37.50, SC37.60 and SC37.66), SC37.21 (identical to SC37.53, SC37.54 and SC37.68), SC37.22, SC37.23, SC37.28 (identical to SC37.32), SC37.29, SC37.37 (identical to and SC37.78), SC37.39, SC37.40, SC37.41, SC37.44 (identical to SC37.46), SC37.45 (identical to SC37.67), SC37.47 (identical to SC37.57), SC37.48, SC37.51, SC37.72, SC37.75, SC37.77, SC37.108, SC37.122, SC37.127, SC37.136 (identical to SC37.208), SC37.141, SC37.150, SC37.160, SC37.163, SC37.169, SC37.170, SC37.177, SC37.185, SC37.191, SC37.193, SC37.196, SC37.202, SC37.212, SC37.223, SC37.226, SC37.231, SC37.233, SC37.236, SC37.239, SC37.243 and SC37.244; and humanized antibodies, termed hSC37.2, hSC37.17, hSC37.39, hSC37.67, and hSC37.67v1.

In addition to the antibodies having identical light and heavy variable regions as denoted in parenthesis following the relevant antibody in the preceding paragraph, there are a number of antibodies that share an identical light chain variable region as follows: SC37.17 (identical light chain to SC37.21, SC37.53, SC37.54, SC37.68), SC37.23 (identical light chain to SC37.28 and SC37.32), SC37.208 (identical light chain to SC37.217, SC37.232, SC37.136 and SC37.172), SC37.122 (identical light chain to SC37.198). In addition there are some antibodies that share an identical heavy chain variable region as follows: SC37.20 (identical heavy chain to SC37.30, SC37.34, SC37.36, SC37.38, SC37.50, SC37.60, SC37.66 and SC37.74), SC37.23 (identical heavy chain to SC37.36), SC37.47 (identical heavy chain to SC37.57 and SC37.75), SC37.122 (identical heavy chain to SC37.127), SC37.160 (identical heavy chain to SC37.198). Notably, many of the above unique murine antibodies comprise lambda light chains. Only unique sequences are presented in FIGS. 7A and 7B i.e. FIGS. 7A and 7B do not contain duplicate sequences.

The amino acid sequences are annotated to identify the framework regions (i.e. FR1-FR4_) and the complementarity determining regions (i.e., CDRL1-CDRL3 in FIG. 7A or CDRH1-CDRH3 in FIG. 7B) defined as per Kabat. The variable region sequences were analyzed using a proprietary version of the Abysis database to provide the CDR and FR designations. Though the CDRs are defined as per Kabat those skilled in art will appreciate that the same database could be used to provide CDR and FR designations as per Chothia or McCallum. FIG. 7C is a table showing nucleic acid sequences encoding the amino acid sequences of the heavy and light chain variable regions set forth in FIGS. 7A and 7B.

### Example 10

### Generation of Chimeric and Humanized Anti-RNF43 Antibodies

Chimeric anti-RNF43 antibodies were generated using art-recognized techniques as follows. Total RNA was extracted from the hybridomas as described in Example 1 and PCR amplified. Data regarding V, D and J gene segments of the VH and VL chains of the following murine antibodies: SC37.2, SC37.17, SC37.39 and SC37.67 were obtained from an analysis of the subject nucleic acid sequences (see FIG. 7C for nucleic acid sequences). Primer sets specific to the framework sequence of the VH and VL chain of the antibodies were designed using the following restriction sites: AgeI and XhoI for the VH fragments, and XmaI and DraIII for the VL fragments. PCR products were purified with a Qiaquick PCR purification kit (Qiagen), followed by digestion with restriction enzymes AgeI and XhoI for the VH fragments and XmaI and DraIII for the VL fragments. The VH and VL digested PCR products were purified and ligated into IgH or I_{D}(expression vectors, respectively. Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the VH fragment was cloned into the AgeI-XhoI restriction sites of the pEE6.4 expression vector (Lonza) comprising HuIgG1 and the VL fragment was cloned into the XmaI-DraIII restriction sites of the pEE12.4 expression vector (Lonza) comprising Hu-Kappa light constant region.

Chimeric antibodies comprising the entire murine heavy and light chain variable regions and human constant regions were expressed by co-transfection of HEK293T cells with pEE6.4HuIgG1 and pEE12.4Hu-Kappa expression vectors. Prior to transfection the HEK293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HuIgG1 and pEE12.4Hu-Kappa vector DNA were added to 50 μL HEK293T transfection reagent in 1.5 mL Opti-MEM. The mix was incubated for 30 mins. at room temperature and plated. Supernatants were harvested three to six days after transfection. Culture supernatants containing recombinant chimeric antibodies were cleared from cell debris by centrifugation at 800×g for 10 mins. and stored at 4° C. Recombinant chimeric antibodies were purified with Protein A beads.

Murine anti-RNF43 antibodies were CDR grafted or humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences, and the framework sequences and CDRs of the relevant mouse antibodies. For the purpose of the analysis the assignment of amino acids to each of the CDR domains was done in accordance with Kabat et al. numbering. In this regard FIGS. 7E to 7H show heavy and light CDRs derived using various analytical schemes for the murine antibodies SC37.2, SC37.17, SC17.39 and SC37.67. Once the variable regions comprising murine Kabat CDRs and the selected human frameworks were designed, they were generated from synthetic gene segments (Integrated DNA Technologies). Humanized antibodies were then cloned and expressed using the molecular methods described above for chimeric antibodies.

The VL and VH amino acid sequences of the humanized antibodies hSC37.2, hSC37.17, hSC17.39, hSC37.67 and hSC37.67v1 (FIGS. 7A and 7B; SEQ ID NOS: 254-270, even numbers) are derived from the VL and VH sequences of the corresponding murine antibodies (e.g. hSC37.2 is derived from SC37.2). The corresponding nucleic acid sequences of the VL and VH are set forth in FIG. 7C (SEQ ID NOS: 253-271, odd numbers). Table 5 shows that very few framework changes were necessary to maintain the favorable properties of the selected antibodies.

For one humanized clone conservative mutations were introduced into the CDRs to address stability concerns. In this regard, a single amino acid change in CDRL3 (N91Q) of the light chain of hSC37.67 led to hSC37.67 variant 1 (hSC37.67v1). For each of the humanized constructs the binding affinities of the antibodies were checked to ensure that they were equivalent to either the corresponding chimeric or murine antibody.

TABLE 5

| | | | | | VH | | | | VK |
|---|---|---|---|---|---|---|---|---|---|
| | | | human | VH FR | CDR | human | human | VK FR | CDR |
| mAb | Isotype | human VH | JH | changes | changes | VK | JK | changes | changes |
| hSC37.2 | IgG1/κ | IGHV3-72*01 | JH6 | D73T | None | IGKV1- | JK2 | None | None |
| | | | | | | 27*01 | | | |
| hSC37.17 | IgG1/κ | IGHV1-46*01 | JH1 | Y27F, | None | IGKV4- | JK1 | None | None |
| | | | | T28N, | | 1*01 | | | |
| | | | | F29I, T30K | | | | | |
| | | | | R94L | | | | | |
| hSC37.17 | IgG1 | IGHV1-46*01 | JH1 | Y27F, | None | IGKV4- | JK1 | None | None |
| ss1 | C220S/κ | | | T28N, | | 1*01 | | | |
| | | | | F29I, T30K | | | | | |
| | | | | R94L | | | | | |
| hSC37.39 | IgG1/κ | IGHV1-46*01 | JH6 | None | None | IGKV3- | JK4 | L46A | None |
| | | | | | | 15*01 | | | |
| hSC37.39 | IgG1 | IGHV1-46*01 | JH6 | None | None | IGKV3- | JK4 | L46A | None |
| ss1 | C220S/κ | | | | | 15*01 | | | |
| hSC37.67 | IgG1/κ | IGHV1-3*01 | JH1 | R71A | None | IGKV1- | JK4 | Y49S | None |
| | | | | A93E | | 39*01 | | F71Y | |
| hSC37.67v1 | IgG1/κ | IGHV1-3*01 | JH1 | R71A | None | IGKV1- | JK4 | Y49S | N91Q |
| | | | | A93E | | 39*01 | | F71Y | |

Following humanization of the above selected antibodies, the resulting VH and VL chain amino acid sequences were analyzed to determine their homology with regard to the murine donor and human acceptor light and heavy chain variable regions. The results, shown in Table 6 below, reveal that the humanized constructs consistently exhibited a higher homology with respect to the human acceptor sequences than with the murine donor sequences. The murine heavy and light chain variable regions show a similar overall percentage homology to a closest match of human germline genes 82%-91% compared with the homology of the humanized antibodies and the donor hybridoma protein sequences 77%-88%.

TABLE 6

| | Homology to Human (CDR | Homology to Murine Parent |
|---|---|---|
| mAb | acceptor) | (CDR donor) |
| hSC37.2 HC | 91% | 85% |
| hSC37.2 LC | 86% | 82% |
| hSC37.17 HC | 83% | 82% |
| hSC37.17 LC | 82% | 85% |
| hSC37.39 HC | 90% | 81% |
| hSC37.39 LC | 85% | 77% |
| hSC37.67 HC | 85% | 80% |
| hSC37.67 LC | 84% | 88% |

### Example 11

### Generation of Site-Specific Anti-RNF43Antibodies

Engineered human IgG1/kappa anti-RNF43 site-specific antibodies were constructed comprising a native light chain (LC) constant region and heavy chain (HC) constant region, wherein cysteine 220 (C220) in the upper hinge region of the HC, which forms an interchain disulfide bond with cysteine 214 (C214) in the LC, was substituted with serine (C220S). When assembled the HCs and LCs form an antibody comprising two free cysteines (at position 214 on the light chain) that are suitable for conjugation to a therapeutic agent. Unless otherwise noted, all numbering of constant region residues is in accordance with the numbering scheme as set forth in Kabat et al.

The engineered antibodies were generated as follows. An expression vector encoding the humanized anti-RNF43 antibody hSC37.17 HC (SEQ ID NO: 274) or hSC37.39 HC (SEQ ID NO: 277), was used as a template for PCR amplification and site directed mutagenesis. Site directed mutagenesis was performed using the Quick-Change® system (Agilent Technologies) according to the manufacturer's instructions.

The vector encoding the mutant C220S HC of hSC37.17 (SEQ ID NO: 275) or hSC37.39 (SEQ ID NO: 278) was co-transfected, respectively, with the native IgG1 kappa LC of hSC37.17 (SEQ ID NO: 273) or the kappa LC of hSC37.39 (SEQ ID NO: 276), in CHO-S cells and expressed using a mammalian transient expression system. The engineered anti-RNF43 site-specific antibodies containing the C220S mutant were termed hSC37.17ss1 and hSC37.39ss1. Amino acid sequences of the full length LC and HC of the hSC37.17ss1 (SEQ ID NOS: 273 and 275) and hSC37.39ss1 (SEQ ID NOS: 276 and 278) site specific antibodies are shown in FIG. 7D. The engineered anti-RNF43 antibodies were characterized by SDS-PAGE to confirm that the correct mutants had been generated. SDS-PAGE was conducted on a pre-cast 10% Tris-Glycine mini gel from life technologies in the presence and absence of a reducing agent such as DTT (dithiothreitol). Following electrophoresis, the gels were stained with a colloidal coomassie solution. Under reducing conditions, two bands corresponding to the free LCs and free HCs, were observed. This pattern is typical of IgG molecules in reducing conditions. Under non-reducing conditions, the band patterns were different from native IgG molecules, indicative of the absence of a disulfide bond between the HC and LC. A band around 98 kD corresponding to the HC-HC dimer was observed. In addition, a faint band corresponding to the free LC and a predominant band around 48 kD that corresponded to a LC-LC dimer was observed. The formation of some amount of LC-LC species is expected due to the free cysteines on the C-terminus of each LC.

### Example 12

### Preparation of Anti-RNF43 Antibody-Drug Conjugates

Anti-RNF43 antibody drug conjugates (ADCs) are prepared having the Ab-[L-D] structure, where Ab refers to the anti-RNF43 antibody, L refers to a linker (e.g. a terminal maleimido moiety with a free sulfhydryl group) and D refers to a drug or cytotoxin (e.g. auristatins, calicheamicin etc). Each ADC comprises an anti-RNF43 antibody covalently linked to a linker-drug. ADCs are synthesized and purified using techniques known in the art, for example, essentially as follows. The cysteine bonds of anti-RNF43 antibodies are partially reduced with a pre-determined molar addition of mol tris(2-carboxyethyl)-phosphine (TCEP) per mol antibody for 90 min. at 20° C. in phosphate buffered saline (PBS) with 5 mM EDTA. The linker-drug, dissolved in dimethyl acetamide (DMA), is added at a ratio of 3 mol/mol anti-RNF43 antibody. The reaction is allowed to proceed for 30 min. Using a 10 mM stock solution of N-acetyl cysteine (NAC) prepared in water, the reaction is quenched with the addition of excess NAC to linker-drug. After a minimum quench time of 20 mins., the pH is adjusted to 6.0 with the addition of 0.5 M acetic acid and buffer exchanged into diafiltration buffer by diafiltration using a 30 kDa membrane. The dialfiltered anti-RNF43 ADC is then formulated with sucrose and polysorbate-20 to the target final concentration. The resulting anti-RNF43 ADCs are analyzed for protein concentration (by measuring UV), aggregation (SEC), drug to antibody ratio (DAR) by reverse-phase HPLC (RP-HPLC) and in vitro cytotoxicity.

### Example 13

### Conjugation of Site Specific Anti-RNF43 Antibodies Using a Selective Reduction Process

Anti-RNF43 antibody drug conjugates (ADCs) are prepared having the Ab-[L-D] structure as described in Example 12 above, wherein the Ab moiety is a site specific antibody, for example, hSC37.17ss1 or hSC37.39ss1, generated as set forth in Example 11 above. The desired product is an ADC that is maximally conjugated on the unpaired cysteine (C214 in the case of IgG1 site specific antibodies or C127 on IgG4 site specific antibodies) on each LC constant region and that minimizes ADCs having a drug to antibody ratio (DAR) which is greater than 2 (DAR>2) or less than 2 (DAR<2) while maximizing ADCs having a DAR of 2 (DAR=2).

In order to further improve the specificity of the conjugation and homogeneity of the final site-specific ADC, the site specific antibody (e.g. “hSC37.17ss1” or “hSC37.39ss1”) is selectively reduced using, for example, a process comprising a stabilizing agent (e.g. L-arginine) and a mild reducing agent (e.g. glutathione) prior to conjugation with the linker-drug, followed by preparative hydrophobic interaction chromatography (HIC) that is used to separate the different DAR species. The above procedures are conducted, for example, essentially as described below.

A preparation of the site specific antibody is partially reduced in a buffer containing 1M L-arginine/5 mM glutathione, reduced (GSH)/5 mM EDTA, pH 8.0 for a minimum of one hour at room temperature. All preparations are then buffer exchanged into a 20 mM Tris/3.2 mM EDTA, pH 8.2 buffer using a 30 kDa membrane (Millipore Amicon Ultra) to remove the reducing buffer. The resulting partially reduced preparations are then conjugated to a cytotoxin (e.g. auristatin, calicheamicin etc.) via a linker (e.g. maleimide linker) for a minimum of 30 mins. at room temperature. The reaction is then quenched with the addition of excess NAC to linker-drug using a 10 mM stock solution of NAC prepared in water. After a minimum quench time of 20 mins., the pH is adjusted to 6.0 with the addition of 0.5 M acetic acid. The site specific ADC is buffer exchanged into diafiltration buffer using a 30 kDa membrane. The site specific ADC preparation is then diluted with a high salt buffer to increase the conductivity to promote binding onto the resin, and then loaded on a Butyl HP resin chromatography column (GE Life Sciences). A decreasing salt gradient is then employed to separate the different DAR species based on hydrophobicity, where DAR=0 species elute first, followed by DAR=1, DAR=2, and then higher DAR species.

The final ADC “HIC purified DAR=2” preparation is analyzed using RP-HPLC to determine the percent conjugation on the HCs and LCs and the DAR distribution. The samples are also analyzed using analytical HIC to determine the amount of DAR=2 species relative to the unwanted DAR>2 and DAR<2 species.

### Example 14

### RNF43 Protein Expression in Tumors

Given the elevated RNF43 mRNA transcript levels associated with various tumors described in Examples 1-3, work was undertaken to test whether RNF43 protein expression was also elevated in PDX tumors. To detect and quantify RNF43 protein expression, an electrochemiluminscence RNF43 sandwich ELISA assay was developed using the MSD Discovery Platform (Meso Scale Discovery).

PDX tumors were excised from mice and flash frozen on dry ice/ethanol. Protein Extraction Buffer (Biochain Institute) was added to the thawed tumor pieces and tumors were pulverized using a TissueLyser system (Qiagen). Lysates were cleared by centrifugation (20,000 g, 20 mins., 4° C.) and the total protein concentration in each lysate was quantified using bicinchoninic acid. The protein lysates were normalized to 5 mg/mL and stored at −80° C. until assayed. Normal tissues were purchased from a commercial source and processed as described above.

RNF43 protein concentrations from the lysate samples were determined by interpolating the values from a standard protein concentration curve that was generated using purified recombinant RNF43 protein with a histidine tag (Sino Biological cat#16108-H08H). The RNF43 protein standard curve and protein quantification assay were conducted as follows:

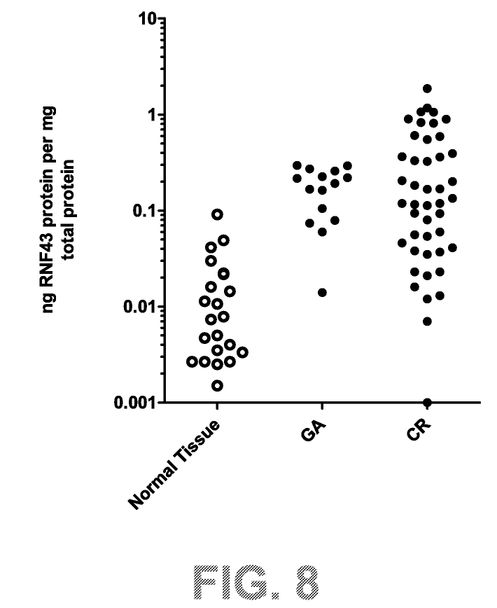

MSD 384 well standard plates were coated overnight at 4° C. with 15 μL of an anti-RNF43 capture antibody at 2 μg/mL in PBS. Plates were washed in PBST and blocked in 35 μL MSD 3% Blocker A solution for one hour while shaking. Plates were again washed in PBST. 10 μL of 5× diluted lysate or serially diluted recombinant RNF43 standard in MSD 1% Blocker A containing 10% Protein Extraction Buffer was added to the wells and incubated for two hours while shaking. Plates were again washed in PBST. The anti-RNF43 detection antibody was then sulfo-tagged using an MSD® SULFO-TAG NHS Ester according to the manufacturer's protocol. 10 μL of the tagged anti-RNF43 detection antibody was added to the washed plates at 0.5 μg/mL in MSD 1% Blocker A for 1 hour at room temperature while shaking. Plates were washed in PBST. MSD Read Buffer T with surfactant was diluted to 1× in water and 35 μL was added to each well. Plates were read on an MSD Sector Imager 2400 using an integrated software analysis program to derive RNF43 concentrations in lysate samples via interpolation from the standard curve. Values were then divided by total protein concentration to yield nanograms of RNF43 per milligram of total lysate protein. The results are shown in FIG. 8 wherein each spot represents RNF43 protein concentrations derived from a single PDX tumor line. While each spot is derived from a single PDX line, in most cases multiple biological samples were tested from the same PDX line and values were averaged to provide the data point.

FIG. 8 shows that representative GA and CR PDX tumor cell lines exhibited high RNF43 protein expression compared to normal tissues. Normal tissues that were tested include adrenal gland, artery, colon, esophagus, gall bladder, heart, kidney, liver, lung, peripheral and sciatic nerve, pancreas, skeletal muscle, skin, small intestine, spleen, stomach, trachea, red and white blood cells and platelets, bladder, brain, breast, eye, lymph node, ovary, pituitary gland, prostate and spinal cord. In the following PDX lines, there was a good correlation between RNF43 protein expression and mRNA expression (determined either by microarray or qPCR): CR88, CR64, CR104, CR76, and CR99. These data, combined with the mRNA transcription data for RNF43 expression set forth above strongly reinforce the proposition that RNF43 determinants provide attractive targets for therapeutic intervention.

### Example 15

### Detection of RNF43 on the Surface of Tumors Using In Situ Hybridization

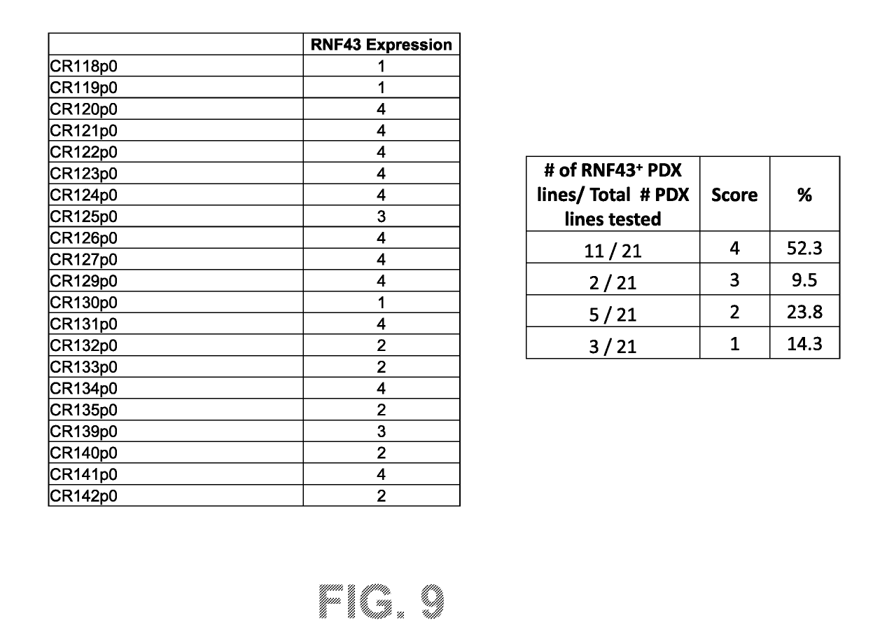

RNA in situ hybridization for RNF43 mRNA was performed using an RNAscope® 2.0 Reagent Kit (Advanced Cell Diagnostics; Wang et al, 2012, PMID: 22166544). The RNAscope probe used for RNF43 was designed between nucleotides 3451-4489. Each sample was quality controlled for RNA integrity with an RNAscope probe specific to Peptidylprolyl Isomerase B (PPIB), a cyclosporine-binding protein located within the endoplasmic reticulum of all cells (data not shown). Background staining was determined using a probe specific to DiAminoPimelate (dapB) RNA (data not shown). Briefly, 5 μm formalin fixed, paraffin embedded (FFPE) tissue sections of 21 primary patient colorectal tumor samples were pretreated with heat and protease prior to hybridization with the RNF43 oligo probes. Preamplifier, amplifier and HRP-labeled oligos were then hybridized sequentially, followed by chromogenic precipitate development with 3,3′-diaminobenzidine. Specific RNA staining signal was identified as brown, punctate dots. The FFPE slides were counterstained with Gill's Hematoxylin and analyzed under a light microscope. Staining was manually scored on a scale of 0 to 4, where 0=no staining or less than 1 dot per 10 cells; 1=1-3 dots per cell; 2=4-10 dots per cell; 3=more than 10 dots per cell and less than 10% positive cells have dots found in clusters; 4=more than 10 dots per cell with more than 10% of positive cells have dots in clusters. FIG. 9 shows that all 21 PDX lines tested, expressed RNF43 to some extent, with 52.3% of the tumors expressing a score of 4.

### Example 16

### Anti-RNF43 Antibodies Detect RNF43 Protein Expression on Tumors Using Flow Cytometry

Flow cytometry was used to assess the ability of the anti-RNF43 antibodies of the invention to specifically detect the presence of anti-RNF43 protein on the surface of CR PDX tumor cell lines.

CR PDX tumor cells were harvested and dissociated using art-recognized enzymatic tissue digestion techniques to obtain single cell suspensions (see, for example, U.S.P.N. 2007/0292414). Tumor cells were incubated for 10 minutes with mouse whole IgG (10 μg/ml in 2% FCS/PBS) to block non-specific antibody binding, then co-stained with fluorescently-conjugated commercially available anti-mouse CD45 and H-2K^{d} antibodies, anti-human EpCAM, and anti-human CD46 and/or CD324 to identify NTG (CD46⁻ cells) and CSC/TIC (CD46^{hi}/CD324⁺ cells) populations (see U.S.P.N.s 2013/0260385, 2013/0061340 and 2013/0061342). Tumor cells were then incubated for 30 mins. with a biotinylated anti-RNF43 antibody (Biotin-SC37.67, 10 μg/ml in 2% FCS/PBS) or with IgG isotype matched control antibodies and washed twice in PBS/2% FCS. The cells were incubated for 15 mins. with APC-labeled streptavidin (1 μg/ml in 2% FCS/PBS), washed twice with 1 mL PBS/2% FCS and re-suspended in PBS/2% FCS with DAPI (to detect dead cells). Antibody binding to live human cells was interpreted as retention of APC signal on mCD45-H2k^{d-}EpCAM⁺DAPI⁻ events as analyzed on a BD FACSCanto II flow cytometer. FIG. 10 shows anti-RNF43 antibodies of the invention (black lines) were able to specifically bind to live human CR PDX tumor cells (CR14, CR81, CR91, CR99, CR104, CR115) with significantly greater intensity than the IgG isotype control antibodies (gray-filled histogram). In addition, in the majority of PDX lines tested, RNF43 expression is higher in CSCs (solid black line) compared to NTGs (dashed black line), indicating that RNF43 expression is associated with tumorigenic populations. FIG. 10 includes a table comparing the staining intensity of the anti-RNF43 antibodies with that of control antibodies, with expression enumerated as the geometric mean fluorescence intensity less the intensity observed with isotype control antibodies (AMFI).

### Example 17

### Anti-RNF43 Antibodies Facilitate Delivery of Cytotoxic Agents

To determine whether anti-RNF43 antibodies of the invention are able to internalize in order to mediate the delivery of cytotoxic agents to cells, an in vitro cell killing assay was performed using selected anti-RNF43 antibodies and saporin linked to a secondary anti-mouse antibody FAB fragment. Saporin is a plant toxin that deactivates ribosomes, thereby inhibiting protein synthesis and resulting in the death of the cell. Saporin is only cytotoxic inside the cell where it has access to ribosomes, but is unable to internalize on its own. Therefore, saporin-mediated cellular cytotoxicity in these assays is indicative of the ability of the anti-mouse FAB-Saporin construct to internalize upon binding and internalization of the associated murine or humanized anti-RNF43 antibodies into the target cells.

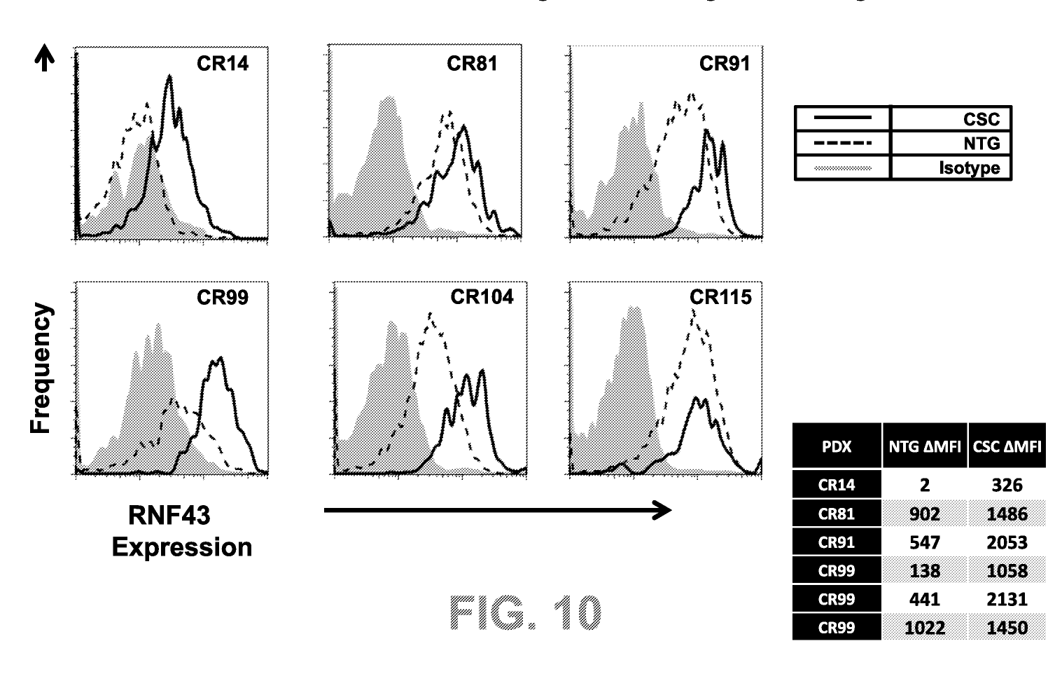

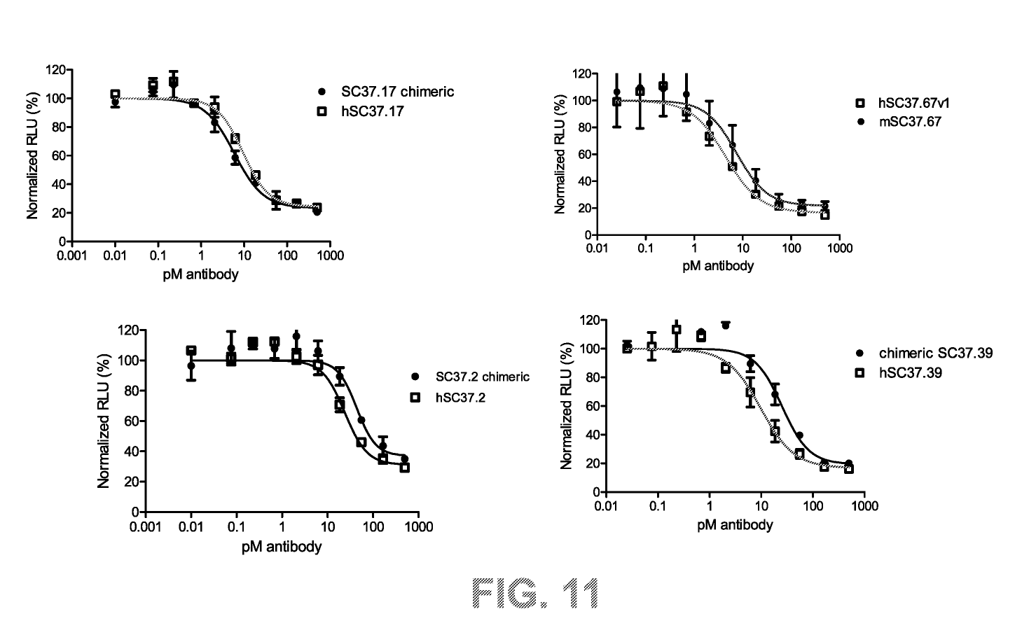

Single cell suspensions of HEK293T cells overexpressing hRNF43 were plated at 500 cells per well into BD Tissue Culture plates (BD Biosciences). One day later, various concentrations of purified anti-RNF43 antibodies (either murine or humanized) were added to the culture together with a fixed concentration of 2 nM anti-mouse IgG FAB-saporin conjugates (Advanced Targeting Systems) (for testing mouse antibodies) or 2 nM anti-human IgG FAB-saporin conjugates (for testing humanized antibodies). Following incubation for 96 hours, viable cells were enumerated using CellTiter-Glo® (Promega) as per the manufacturer's instructions. Raw luminescence counts using cultures containing cells incubated only with the secondary FAB-saporin conjugate were set as 100% reference values and all other counts were calculated as a percentage of the reference value. A large subset of anti-RNF43 murine antibodies at a concentration of 250 pM effectively killed HEK293T cells overexpressing hRNF43 with varying efficacy (FIG. 5A), whereas the mouse IgG2b isotype control antibody (mIgG2b) at the same concentration did not (data not shown). In addition, the anti-RNF43 humanized antibodies (hSC37.2, hSC37.17, hSC37.39, and hSC37.67v1) effectively killed HEK293T cells overexpressing RNF43. The humanized antibodies showed comparable efficacy to the chimeric antibody (in the case of hSC37.2, hSC37.17 and hSC37.39) or murine antibody (in the case of hSC37.67v1) from which they were derived (FIG. 11).

The above results demonstrate the ability of anti-RNF43 antibodies to mediate internalization and their ability to deliver cytotoxic payloads, supporting the hypothesis that anti-RNF43 antibodies may have therapeutic utility as the targeting moiety of an ADC.

### Example 18

### Reduction of Tumor Initiating Cell Frequency by Anti-RNF43 Antibody-Drug Conjugates

As demonstrated in Example 1 above RNF43 expression is associated with cancer stem cells. Accordingly, to demonstrate that treatment with anti-RNF43 ADCs reduces the frequency of tumor initiating cells (TIC) that are known to be drug resistant and to fuel tumor recurrence and metastasis, in vivo limiting dilution assays (LDA) are performed, for example, essentially as described below.

PDX tumors (e.g. colorectal) are grown subcutaneously in immunodeficient mice. When tumor volumes average 150 mm³-250 mm³ in size, the mice are randomly segregated into two groups. One group is injected intraperitoneally with a human IgG1 conjugated to a drug as a negative control; and the other group is injected intraperitoneally with an anti-RNF43 ADC (e.g., as prepared in Examples 16 and 18). One week following dosing, two representative mice from each group are euthanized and their tumors are harvested and dispersed to single-cell suspensions. The tumor cells from each treatment group are then harvested, pooled and disaggregated as previously described in Example 1. The cells are labeled with FITC conjugated anti-mouse H2kD and anti-mouse CD45 antibodies to detect mouse cells; EpCAM to detect human cells; and DAPI to detect dead cells. The resulting suspension is then sorted by FACS using a BD FACS Canto II flow cytometer and live human tumor cells are isolated and collected.

Four cohorts of mice are injected with either 1250, 375, 115 or 35 sorted live, human cells from tumors treated with anti-RNF43 ADC. As a negative control four cohorts of mice are transplanted with either 1000, 300, 100 or 30 sorted live, human cells from tumors treated with the control IgG1 ADC. Tumors in recipient mice are measured weekly, and individual mice are euthanized before tumors reach 1500 mm³. Recipient mice are scored as having positive or negative tumor growth. Positive tumor growth is defined as growth of a tumor exceeding 100 mm³.

Poisson distribution statistics (L-Calc software, Stemcell Technologies) is used to calculate the frequency of TICs in each population.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. An antibody drug conjugate of the formula M-[L-D]n, or a pharmaceutically acceptable salt thereof, wherein
M comprises an anti-RNF43 antibody;
L comprises a linker;
D comprises a cytotoxin; and
n is an integer from 1 to 20.

2. The antibody drug conjugate of claim 1, wherein the anti-RNF43 antibody is an internalizing antibody.

3. The antibody drug conjugate of claim 1, wherein the anti-RNF43 antibody is a chimeric, CDR grafted or humanized antibody, or fragment thereof.

4. The antibody drug conjugate of claim 1, wherein the anti-RNF43 antibody binds to tumor initiating cells.

5. The antibody drug conjugate of claim 1, wherein the anti-RNF43 antibody binds to human RNF43 (SEQ ID NO: 5) and does not bind to human ZNRF3 (SEQ ID NO: 6).

6. The antibody drug conjugate of claim 1, wherein the anti-RNF43 antibody binds to human RNF43 (SEQ ID NO: 5) on the surface of a eukaryotic cell wherein the binding of the antibody decreases WNT signaling.

7. The antibody drug conjugate of claim 1, wherein the anti-RNF43 antibody binds to human RNF43 (SEQ ID NO: 5) on the surface of a eukaryotic cell wherein the binding of the antibody increases WNT signaling.

8. The antibody drug conjugate of claim 1, wherein the anti-RNF43 antibody binds to human RNF43 (SEQ ID NO: 5) on the surface of a eukaryotic cell wherein the binding of the antibody does not affect WNT signaling.

9. The antibody drug conjugate of claim 1 wherein the anti-RNF43 antibody binds to human RNF43 (SEQ ID NO: 5) and blocks binding of R-spondin to RNF43.

10. The antibody drug conjugate of claim 1 wherein the anti-RNF43 antibody binds to human RNF43 (SEQ ID NO: 5) and does not block binding of R-spondin to RNF43.

11. The antibody drug conjugate of claim 1, wherein the anti-RNF43 antibody binds to human RNF43 (SEQ ID NO: 5) on the surface of a eukaryotic cell wherein the binding of the antibody does not block R-spondin-stimulated WNT signaling.

12. The antibody drug conjugate of claim 1, wherein the anti-RNF43 antibody binds to human RNF43 (SEQ ID NO: 5) on the surface of a eukaryotic cell wherein the binding of the antibody blocks R-spondin-stimulated WNT signaling.

13. An isolated antibody that binds to human RNF43 (SEQ ID NO: 5) and competes for binding to human RNF43 with an antibody comprising:
(1) a light chain variable region set forth as SEQ ID NO: 78 and a heavy chain variable region set forth as SEQ ID NO: 80; or
(2) a light chain variable region set forth as SEQ ID NO: 110 and a heavy chain variable region set forth as SEQ ID NO: 112.

14. An isolated antibody that binds to human RNF43 (SEQ ID NO: 5) comprising a light chain comprising the following light chain complimentarity determining regions (CDRL): CDRL1: SEQ ID NO: 288; CDRL2: SEQ ID NO: 289; CDRL3: SEQ ID NO: 290; and a heavy chain comprising the following heavy chain complimentarity determining regions (CDRH): CDRH1: SEQ ID NO: 291; CDRH2: SEQ ID NO: 292; CDRH3: SEQ ID NO: 293.

15. An isolated antibody that binds to human RNF43 (SEQ ID NO: 5) comprising a light chain comprising the following light chain complimentarity determining regions (CDRL): CDRL1: SEQ ID NO: 294; CDRL2: SEQ ID NO: 295; CDRL3: SEQ ID NO: 296; and a heavy chain comprising the following CDRH: CDRH1: SEQ ID NO: 297; CDRH2: SEQ ID NO: 298; CDRH3: SEQ ID NO: 299.

16. A humanized antibody that binds to human RNF43 (SEQ ID NO: 5) comprising a light chain set forth as SEQ ID NO: 273; and a heavy chain set forth as SEQ ID NO: 275.

17. A humanized antibody that binds to human RNF43 (SEQ ID NO: 5) comprising a light chain set forth as SEQ ID NO: 276; and a heavy chain variable region set forth as SEQ ID NO: 278.

18. A nucleic acid encoding a light chain set forth as SEQ ID NO: 273 or 276, or a heavy chain set forth as SEQ ID NO: 275 or 278.

19. A vector comprising the nucleic acid of claim 18.

20. A host cell comprising the vector of claim 19.

21. A pharmaceutical composition comprising an ADC of any one of claims 1 to 11.

22. A method of treating cancer comprising administering a pharmaceutical composition of claim 21 to a subject in need thereof.

23. The method of claim 22, wherein the cancer is selected from colorectal cancer or lung cancer.


SEQUENCE LISTING (306 sequences)

SEQ ID NO: 1 - protein, 107 aa, Homo sapiens [sequence omitted]

SEQ ID NO: 2 - protein, 329 aa, Homo sapiens
  misc_feature: IgGI heavy chain (HC) constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 197 aa, Homo sapiens
  misc_feature: RNF43 extra-cellular domain
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDY

SEQ ID NO: 4 - protein, 219 aa, Homo sapiens
  misc_feature: ZNRF43 extra-cellular domain
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDM

SEQ ID NO: 5 - protein, 783 aa, Homo sapiens
  misc_feature: RNF43
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 6 - protein, 936 aa, Homo sapiens
  misc_feature: ZNRF43
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 7-20 - skipped (14 entries)

SEQ ID NO: 21 - DNA, 339 bp, Mus musculus
  misc_feature: SC37.1 Light Chain Variable Region
gacattgtgatgtcacagtctccatcctccctacctgtgtcagttggagagagggttactatgagctgcaagtccagtcagagccttttatatagtaccaatcaaaaaaactacttggcctggtaccagcagaaaccagggcagtctcctaaactgctgatttactgggcatccactagggaatctggggtccctgatcgcttcacaggcagtggatctgggacagatttcactctcaccatcagcggtgtgaaggctgaagacctggcagtttattactgtcagcaatattataactatcctcggacgttcggtggaggcaccaagctggaaatcaaa

SEQ ID NO: 22 - protein, 113 aa, Mus musculus
  misc_feature: SC37.1 Light Chain Variable Region
DIVMSQSPSSLPVSVGERVTMSCKSSQSLLYSTNQKNYLAWYQQKPGQSPKLLIYWASTRESGVPDRFTGSGSGTDFTLTISGVKAEDLAVYYCQQYYNYPRTFGGGTKLEIK

SEQ ID NO: 23 - DNA, 363 bp, Mus musculus
  misc_feature: SC37.1 Heavy Chain Variable Region
caggtccagttgcagcagcctggggcggagctggtgaggcctggggcttcagtgaagctgtcctgcaaggtttctggctacaccttcgccagctactggatgaactgggtgaagcagaggcctggacaaggccttgaatggattgctatgattgattattcagacagtgaaactcactacaatcaaatgttcaaggacaaggccacattgactgtagacaaatcctccaacacagcctacatgcagctcagcagcctgacatctgaggactctgcggtctattactgtgcaagagagggtaactacggtaacccctcctactttgacttctggggccaaggcaccactctcacagtctcctca

SEQ ID NO: 24 - protein, 121 aa, Mus musculus
  misc_feature: SC37.1 Heavy Chain Variable Region
QVQLQQPGAELVRPGASVKLSCKVSGYTFASYWMNWVKQRPGQGLEWIAMIDYSDSETHYNQMFKDKATLTVDKSSNTAYMQLSSLTSEDSAVYYCAREGNYGNPSYFDFWGQGTTLTVSS

SEQ ID NO: 25 - DNA, 339 bp, Mus musculus
  misc_feature: SC37.2 Light Chain Variable Region
gacattgtgatgtcacagtctccatcctccctagctgtgtcagttggagagaaggttactatgagctgcaagtccagtcagacccttttatattatagcaatcaaaagaactacttggcctggtaccagcggaaaccagggctgtctcctaaactgctgatttactgggcatccactagggattctggggtccctgatcgcttcacaggcagtggatctgggacagatttcactctcaccatcagcagtgtgaaggctgaagacctggcagtttattactgtcagcaatattataactatccgctcacgttcggtgctgggaccaagctggagctgaaa

SEQ ID NO: 26 - protein, 113 aa, Mus musculus
  misc_feature: SC37.2 Light Chain Variable Region
DIVMSQSPSSLAVSVGEKVTMSCKSSQTLLYYSNQKNYLAWYQRKPGLSPKLLIYWASTRDSGVPDRFTGSGSGTDFTLTISSVKAEDLAVYYCQQYYNYPLTFGAGTKLELK

SEQ ID NO: 27 - DNA, 360 bp, Mus musculus
  misc_feature: SC37.2 Heavy Chain Variable Region
gaggtgaatctggtggaatctggaggaggcttggtacagcctgggggttctctgagactctcctgtgcaacttctgggttcaccttcagtgatttcgacatggagtgggtccgccagcctccagggaagagactggagtggattgctgcaagtagaaacaaagctaatgattatacaacacaatacagtgcttctgtgaagggtcggttcatcgtctccagagacacttcccaaagcatcgtctacattcagatgaatgccctgagagctgaggacactgccatttattactgtgcaagagataggtacgactatgctttggactactggggtcaaggaacctcagtcaccgtctcctca

SEQ ID NO: 28 - protein, 120 aa, Mus musculus
  misc_feature: SC37.2 Heavy Chain Variable Region
EVNLVESGGGLVQPGGSLRLSCATSGFTFSDFDMEWVRQPPGKRLEWIAASRNKANDYTTQYSASVKGRFIVSRDTSQSIVYIQMNALRAEDTAIYYCARDRYDYALDYWGQGTSVTVSS

SEQ ID NO: 29 - DNA, 327 bp, Mus musculus
  misc_feature: SC37.3 Light Chain Variable Region
caggctgttgtgactcaggaatctgcactcaccacatcacctggtgaaacagtcacattcacttgtcgctcaagtactggggctgttacaacttctaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaatcattgggtgttcggtggaggaaccaaactgactgtccta

SEQ ID NO: 30 - protein, 109 aa, Mus musculus
  misc_feature: SC37.3 Light Chain Variable Region
QAVVTQESALTTSPGETVTFTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 31 - DNA, 351 bp, Mus musculus
  misc_feature: SC37.3 Heavy Chain Variable Region
cagatccagttggttcagtctggacctgagctgaagaagcctggagagacagtcaagatctcctgcaaggcttctgggtataccttcacaaactatggaatgaactgggtgaggcaggctccaggaaagggtttaaagtgggtgggctggataaacaccaacactggagagccaacatatgctgatgacttcaagggacggtttgccttctctttggaaacctctgccagcactgcctatttgcagatcagcaacctcaaaaatgaggacatgtctacatatttctgtgcaggatctcatgattactcctttgcttactggggccaagggactctggtcactgtctctgca

SEQ ID NO: 32 - protein, 117 aa, Mus musculus
  misc_feature: SC37.3 Heavy Chain Variable Region
QIQLVQSGPELKKPGETVKISCKASGYTFTNYGMNWVRQAPGKGLKWVGWINTNTGEPTYADDFKGRFAFSLETSASTAYLQISNLKNEDMSTYFCAGSHDYSFAYWGQGTLVTVSA

SEQ ID NO: 33 - DNA, 318 bp, Mus musculus
  misc_feature: SC37.4 Light Chain Variable Region
gatatccagatgacacaaactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggaacaagatgatattgccacttacttttgccaacagggtaatacgctttacacgttcggaggggggaccaagctggaaataaaa

SEQ ID NO: 34 - protein, 106 aa, Mus musculus
  misc_feature: SC37.4 Light Chain Variable Region
DIQMTQTTSSLSASLGDRVTISCRASQDISNYLNWYQQKPDGTVKLLIYYTSRLHSGVPSRFSGSGSGTDYSLTISNLEQDDIATYFCQQGNTLYTFGGGTKLEIK

SEQ ID NO: 35 - DNA, 378 bp, Mus musculus
  misc_feature: SC37.4 Heavy Chain Variable Region
caggttactctgaaagagtctggccctgggatattgcagccctcccagaccctcagtctgacttgttctttctctgggttttcactgagcacttctggtatgggtgtaggctggattcgtcagccttcagggaagggtctggagtggctggcacacatttggtgggatgatgacaagcgctataaagcagccctgaagagccgactgacaatatccaaggacacctccagcaagcgggtattcctcaagatcgccagtgtggacactgcagatactgccacatacttctgtgctcgaataggaggttacaacggtggtaggttctattactatgccatggacaactggggtcaaggaacctcagtcaccgtctcctca

SEQ ID NO: 36 - protein, 126 aa, Mus musculus
  misc_feature: SC37.4 Heavy Chain Variable Region
QVTLKESGPGILQPSQTLSLTCSFSGFSLSTSGMGVGWIRQPSGKGLEWLAHIWWDDDKRYKAALKSRLTISKDTSSKRVFLKIASVDTADTATYFCARIGGYNGGRFYYYAMDNWGQGTSVTVSS

SEQ ID NO: 37 - DNA, 327 bp, Mus musculus
  misc_feature: SC37.6 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgttctatggtacagcaaccattgggtgttcggtggaggaaccaaactgactgtccta

SEQ ID NO: 38 - protein, 109 aa, Mus musculus
  misc_feature: SC37.6 Light Chain Variable Region
QAVVTQESALTTSPGETVTLTCRSSTGAVTTSNYANWVQEKPDHLFTGLIGGTNNRAPGVPARFSGSLIGDKAALTITGAQTEDEAIYFCVLWYSNHWVFGGGTKLTVL

SEQ ID NO: 39 - DNA, 351 bp, Mus musculus
  misc_feature: SC37.6 Heavy Chain Variable Region
cagatccagttggtgcagtctggacctgagatgaagaagcctggagagacagtcaagatctcctgcaaggcttctgggtataccttcacaaactatggattgaactgggtgaggcaggctccaggaaagggtttaaagtggatgggctggataaacaccaacactggagagccaacatatggtgatgacttcaagggacggtttgccttctcattggaaacctctgccagcactgcctatttacagatcaacaacctcaaaaatgaggacacgacaacatatttctgtgcaagatcctatgattactcgtttgcttattggggccaagggactttggtcactgtctctgca

SEQ ID NO: 40 - protein, 117 aa, Mus musculus
  misc_feature: SC37.6 Heavy Chain Variable Region
QIQLVQSGPEMKKPGETVKISCKASGYTFTNYGLNWVRQAPGKGLKWMGWINTNTGEPTYGDDFKGRFAFSLETSASTAYLQINNLKNEDTTTYFCARSYDYSFAYWGQGTLVTVSA

SEQ ID NO: 41 - DNA, 321 bp, Mus musculus
  misc_feature: SC37.7 Light Chain Variable Region
gacattgtgatgacccagtctcacaaattcatgtccacatcagcaggagacagggtcagcatcacctgcaaggccagtcaggatgtgggtactgctgtagcctggtatcaacagaaaccagggcaatctcctaaaatactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaa

SEQ ID NO: 42 - protein, 107 aa, Mus musculus
  misc_feature: SC37.7 Light Chain Variable Region
DIVMTQSHKFMSTSAGDRVSITCKASQDVGTAVAWYQQKPGQSPKILIYWASTRHTGVPDRFTGSGSGTDFTLTISNVQSEDLADYFCQQYSSYPLTFGAGTKLELK

SEQ ID NO: 43 - DNA, 363 bp, Mus musculus
  misc_feature: SC37.7 Heavy Chain Variable Region
gatgtgcagcttcaggagtcgggacctggcctggtgaaaccttctcagtctctgtccctcacctgcactgtcactgcctactctgtcaccagtgattatgcctggaactggatccggcagtttccaggaaacacactggagtggatgggctacataacctacagtggtagcactgtctacaacccctctctcaaaagtcgaatctctatcactcgagacacatccaagaaccagttcttcctgcagttgaattctgtgactgctgaggacacagccacatattactgtgcaagatctgaagtgggacgagggggctattctatggagtactggggtcagggaacctcagtcaccgtctcctca

SEQ ID NO: 44 - protein, 121 aa, Mus musculus
  misc_feature: SC37.7 Heavy Chain Variable Region
DVQLQESGPGLVKPSQSLSLTCTVTAYSVTSDYAWNWIRQFPGNTLEWMGYITYSGSTVYNPSLKSRISITRDTSKNQFFLQLNSVTAEDTATYYCARSEVGRGGYSMEYWGQGTSVTVSS

SEQ ID NO: 45 - DNA, 327 bp, Mus musculus
  misc_feature: SC37.8 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacagggctgccctcaccatcacaggggcacagactgaggatgagacaatatatttctgtgctctatggctcagcaaccattgggtgttcggtggtggaaccaaactgactgtccta

SEQ ID NO: 46 - protein, 109 aa, Mus musculus
  misc_feature: SC37.8 Light Chain Variable Region
QAVVTQESALTTSPGETVTLTCRSSTGAVTTSNYANWVQEKPDHLFTGLIGGTNNRAPGVPARFSGSLIGDRAALTITGAQTEDETIYFCALWLSNHWVFGGGTKLTVL

SEQ ID NO: 47 - DNA, 351 bp, Mus musculus
  misc_feature: SC37.8 Heavy Chain Variable Region
cagatccagttggtgcagtctggacctgagctgaagaagcctggagagacagtcaagatctcctgcaaggcttctgggtataccttcacaaactatggaatgaactgggtgaagcaggctccaggaaaggttttaaagtggatgggctggataaacacccacactggagagccaacatatgctgatgacttcaagggacggtttgccttctctttggaaacctctgccagcactgcctatttgcagatcaacaacctcaaaaatgaggacatggctacatatttctgtgcaggatcttatgattactcctttgcttactggggccaagggactctggtcactgtctctgca

SEQ ID NO: 48 - protein, 117 aa, Mus musculus
  misc_feature: SC37.8 Heavy Chain Variable Region
QIQLVQSGPELKKPGETVKISCKASGYTFTNYGMNWVKQAPGKVLKWMGWINTHTGEPTYADDFKGRFAFSLETSASTAYLQINNLKNEDMATYFCAGSYDYSFAYWGQGTLVTVSA

SEQ ID NO: 49 - DNA, 327 bp, Mus musculus
  misc_feature: SC37.9 Light Chain Variable Region
caggctgttgtgactcaggaatctgcactcaccacatcacctggtgaaacagtcacactcacttgtcgctcaactactggggctgttacaactagtaactatgccaactgggtccaagaaaaaccagatcatttattcactggtctaatgggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 50 - protein, 109 aa, Mus musculus
  misc_feature: SC37.9 Light Chain Variable Region
QAVVTQESALTTSPGETVTLTCRSTTGAVTTSNYANWVQEKPDHLFTGLMGGTNNRAPGVPARFSGSLIGDKAALTITGAQTEDEAIYFCALWYSNHWVFGGGTKLTVL

SEQ ID NO: 51 - DNA, 351 bp, Mus musculus
  misc_feature: SC37.9 Heavy Chain Variable Region
cagatccagttggtgcagtctggacctgagctgaagaagcctggagagacagtcaagatctcctgcaaggctcctgggtataccttcagaaactatggattgaactgggtgaagcaggctccaggaaagggtttaaagtggatgggctggataaacactaacactggagagccaacatatgctgatgacttcaagggacggtttgccttctctttggatacctctgccagcactgcctatttgcagatcaataacctcaaaaatgaggacatggcaacatatttctgtgcaagatctcatgatttctcctttgtttattggggccaagggactctggtcactgtctctgca

SEQ ID NO: 52 - protein, 117 aa, Mus musculus
  misc_feature: SC37.9 Heavy Chain Variable Region
QIQLVQSGPELKKPGETVKISCKAPGYTFRNYGLNWVKQAPGKGLKWMGWINTNTGEPTYADDFKGRFAFSLDTSASTAYLQINNLKNEDMATYFCARSHDFSFVYWGQGTLVTVSA

SEQ ID NO: 53 - DNA, 327 bp, Mus musculus
  misc_feature: SC37.10 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccgacaaccgagctccaggtgttcctgccagattctcaggctccctgattggagacagggctgccctcaccatcacaggggcacagactgaggatgcggcaatatatttctgtgctctatggtccagcaaccattgggtgttcggtggaggaaccaaactgactgtccta

SEQ ID NO: 54 - protein, 109 aa, Mus musculus
  misc_feature: SC37.10 Light Chain Variable Region
QAVVTQESALTTSPGETVTLTCRSSTGAVTTSNYANWVQEKPDHLFTGLIGGTDNRAPGVPARFSGSLIGDRAALTITGAQTEDAAIYFCALWSSNHWVFGGGTKLTVL

SEQ ID NO: 55 - DNA, 351 bp, Mus musculus
  misc_feature: SC37.10 Heavy Chain Variable Region
cagatccagttggtgcagtctggacctgagctgaagaagcctggagagacagtcgatatctcctgcaaggcctctgggtataccttcacaaactatggcatgaactgggtgaagcaggctccaggaaagggtttaaagtggatgggctggataaacacccacactggagagccaacatatagtgatgatttcaagggacggtttgccttctctttggaaacctctgccagcactgcctacttgcagatcaacaacctcgaaaatgaggacatggctacatatttctgtgcaggatcttatgattactcctttgcttactggggccaagggactctggtcactgtctctgca

SEQ ID NO: 56 - protein, 117 aa, Mus musculus
  misc_feature: SC37.10 Heavy Chain Variable Region
QIQLVQSGPELKKPGETVDISCKASGYTFTNYGMNWVKQAPGKGLKWMGWINTHTGEPTYSDDFKGRFAFSLETSASTAYLQINNLENEDMATYFCAGSYDYSFAYWGQGTLVTVSA

SEQ ID NO: 57 - DNA, 324 bp, Mus musculus
  misc_feature: SC37.11 Light Chain Variable Region
gaaaatgtgctcacccagtctccagcaatcatgtctgcatctccaggggaaaaggtcaccatgacctgcagggccagctcaagtgtaagatccagttactttcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttacccactcacgttcggctcggggacaaagttggaaataaaa

SEQ ID NO: 58 - protein, 108 aa, Mus musculus
  misc_feature: SC37.11 Light Chain Variable Region
ENVLTQSPAIMSASPGEKVTMTCRASSSVRSSYFHWYQQKSGASPKLWIYSTSNLASGVPARFSGSGSGTSYSLTISSVEAEDAATYYCQQYSGYPLTFGSGTKLEIK

SEQ ID NO: 59 - DNA, 348 bp, Mus musculus
  misc_feature: SC37.11 Heavy Chain Variable Region
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggactaatatgggctggtggaagcacttattataattcggctctcatgtccagactgaccatcagaaaagacaactccaggagccaagttttcttaaaaatgaacagtctgcaaactgatgacacagccatgtactactgtgccagagatgactatggtaaatctgcttactggggccaagggactctggtcactgtctctgca

SEQ ID NO: 60 - protein, 116 aa, Mus musculus
  misc_feature: SC37.11 Heavy Chain Variable Region
QVQLKESGPGLVAPSQSLSITCTVSGFSLTSYGVHWVRQPPGKGLEWLGLIWAGGSTYYNSALMSRLTIRKDNSRSQVFLKMNSLQTDDTAMYYCARDDYGKSAYWGQGTLVTVSA

SEQ ID NO: 61 - DNA, 339 bp, Mus musculus
  misc_feature: SC37.12 Light Chain Variable Region
gacattgtgatgacacagtctccatcctccctggctatgtcagtaggacagaaggtcactatgagctgcaagtccagtcagagccttttaaatagttacagtcaaaagaactatttggcctggtaccagcagaaaccaggacagtctcctaaacttctgatatactttgcatccactagggaatctggggtccctgatcgcttcataggcagtggatctgggacagatttcactcttaccatcagcagtgtgcaggctgaagacctggcagattacttctgtcagcaacattataccactcctctcacgttcggtgctgggaccaagctggagctgaaa

SEQ ID NO: 62 - protein, 113 aa, Mus musculus
  misc_feature: SC37.12 Light Chain Variable Region
DIVMSQSPSALAVSVGEKITMSCKCSQSLLHSRDQKNYLAWYQQKPGQSPKLLIYWASTRESGVPDRFTGSGSGTDFTLTINTVKAEDLAVYYCQQHYRYPLTFGAGTKLELK

SEQ ID NO: 63 - DNA, 369 bp, Mus musculus
  misc_feature: SC37.12 Heavy Chain Variable Region
caagttactctaaaagagtctggccctgggatattgaagccctcacagaccctcagtctgacttgttctttctctgggttttcactgagcactcctggtatgggtataggctggattcgtcagccttcagggaagggtctggagtggctggcacacatttggtgggatgatgataagtactataacccatccctgaagagccagctcacaatctccaaggatacctccggaaaccaggtattcctcaagatcaccagtgtggacactgcagaaacttccacttactactgtgctcgaagagctgactacggtagtagcccctggtacttcgatgtctggggcgcagggaccacggtcaccgtctcctca

SEQ ID NO: 64 - protein, 123 aa, Mus musculus
  misc_feature: SC37.12 Heavy Chain Variable Region
QVTLKESGPGILKPSQTLSLTCSFSGFSLSTPGMGIGWIRQPSGKGLEWLAHIWWDDDKYYNPSLKSQLTISKDTSGNQVFLKITSVDTAETSTYYCARRADYGSSPWYFDVWGAGTTVTVSS

SEQ ID NO: 65 - DNA, 336 bp, Mus musculus
  misc_feature: SC37.13 Light Chain Variable Region
gacattgtgatgtcacagtctccatcctccctagctgtgtcagttggagagaaggtttctatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataactattggacgttcggtggaggcaccaagctggaaatcaaa

SEQ ID NO: 66 - protein, 112 aa, Mus musculus
  misc_feature: SC37.13 Light Chain Variable Region
DIVMSQSPSSLAVSVGEKVSMSCKSSQSLLYSSNQKNYLAWYQQKPGQSPKLLIYWASTRESGVPDRFTGSGSGTDFTLTISSVKAEDLAVYYCQQYYNYWTFGGGTKLEIK

SEQ ID NO: 67 - DNA, 363 bp, Mus musculus
  misc_feature: SC37.13 Heavy Chain Variable Region
caggtgcagctgaaggagtcaggacctggcctggtggcgccctcacagaccctgtccatcacttgcactgtctctgggttttcattaaccaactatggtgtacactgggttcgccagcctccaggaaagggtctggagtggctgggactaatatgggctgatgaaaacacaaattataattcggctctcatgtccagactgagcatcagcaaagacaactccaagagccaagttttcttaaaaatgaacagtctgcaaactgatgacacagccatgtactactgtgccagaagggggaggtacgacgacgggcccggggctatggactactggggtcaaggaacctcagtcaccgtctcctca

SEQ ID NO: 68 - protein, 121 aa, Mus musculus
  misc_feature: SC37.13 Heavy Chain Variable Region
QVQLKESGPGLVAPSQTLSITCTVSGFSLTNYGVHWVRQPPGKGLEWLGLIWADENTNYNSALMSRLSISKDNSKSQVFLKMNSLQTDDTAMYYCARRGRYDDGPGAMDYWGQGTSVTVSS

SEQ ID NO: 69 - DNA, 333 bp, Mus musculus
  misc_feature: SC37.15 Light Chain Variable Region
gacattgtgctcacccaatctccagcttctttggctgtgtctctagggcagagagccaccatctcctgcagagccagtgaaagtgttgaatattatggcacaagtttaatgcagtggtaccaacagaaaccaggacagccacccaagctcctcatctatggtgcatccaacgtagaatctggggtccctgccaggtttagtggcagtgggtctgggacagacttcagcctcaacatccatcctgtggaggaggatgatattgcaatgtatatctgtcagcaaagtaggaaggttccgtggacgttcggtggaggcaccaagctggaaatcaaa

SEQ ID NO: 70 - protein, 111 aa, Mus musculus
  misc_feature: SC37.15 Light Chain Variable Region
DIVLTQSPASLAVSLGQRATISCRASESVEYYGTSLMQWYQQKPGQPPKLLIYGASNVESGVPARFSGSGSGTDFSLNIHPVEEDDIAMYICQQSRKVPWTFGGGTKLEIK

SEQ ID NO: 71 - DNA, 345 bp, Mus musculus
  misc_feature: SC37.15 Heavy Chain Variable Region
gaggtccagctgcaacagtttggaactgagctggtgaagcctgggacttcagtgaagatatcctgcaaggcttctggctacacattcactgactacaacatggactgggtgaagcagagccatggaaagagccttgagtggattggagattttaatcctagctatgatagtactacctacaaccagaagttcaagggaaaggccacattgactgttgacaagtcctccagcacagcctacatggagctccgcagcctgacatctgaagacactgcagtctattactgtgcaagtgggcgtgctatggactactggggtcaaggaacctcagtcaccgtctcctca

SEQ ID NO: 72 - protein, 115 aa, Mus musculus
  misc_feature: SC37.15 Heavy Chain Variable Region
EVQLQQFGTELVKPGTSVKISCKASGYTFTDYNMDWVKQSHGKSLEWIGDFNPSYDSTTYNQKFKGKATLTVDKSSSTAYMELRSLTSEDTAVYYCASGRAMDYWGQGTSVTVSS

SEQ ID NO: 73 - DNA, 327 bp, Mus musculus
  misc_feature: SC37.16 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgaactccaggtgttcctgccagattctcaggctccctgattggagacaaggctgccctcaccatcacagggacacagactgaggatgaggcaatatatttctgtgctctatggtacagcaaccattgggtgttcggtggaggaaccaaactgactgtccta

SEQ ID NO: 74 - protein, 109 aa, Mus musculus
  misc_feature: SC37.16 Light Chain Variable Region
QAVVTQESALTTSPGETVTLTCRSSTGAVTTSNYANWVQEKPDHLFTGLIGGTNNRTPGVPARFSGSLIGDKAALTITGTQTEDEAIYFCALWYSNHWVFGGGTKLTVL

SEQ ID NO: 75 - DNA, 357 bp, Mus musculus
  misc_feature: SC37.16 Heavy Chain Variable Region
caggttcagctgcagcagtctggagctgagctgatgaagcccggggcctcagtgaagatatcctgcaaggctactggctacaaattcagtaggtactggatagagtgggtaaagcagaggcctggacatggccttgagtggattggagagatggtacctggaactggtagtattaaatacaatgagaagttcaagggcaaggccacaatcactgcagatacatcctccaccacagcctacatgcaactcagtagcctgacatctgaggactctggcgtctattactgtgcaagatccggccattactacgcctactttgactactggggccaaggctccactctcacagtctcctca

SEQ ID NO: 76 - protein, 119 aa, Mus musculus
  misc_feature: SC37.16 Heavy Chain Variable Region
QVQLQQSGAELMKPGASVKISCKATGYKFSRYWIEWVKQRPGHGLEWIGEMVPGTGSIKYNEKFKGKATITADTSSTTAYMQLSSLTSEDSGVYYCARSGHYYAYFDYWGQGSTLTVSS

SEQ ID NO: 77 - DNA, 333 bp, Mus musculus
  misc_feature: SC37.17 Light Chain Variable Region
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaagctcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 78 - protein, 111 aa, Mus musculus
  misc_feature: SC37.17 Light Chain Variable Region
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPLTFGAGTKLELK

SEQ ID NO: 79 - DNA, 354 bp, Mus musculus
  misc_feature: SC37.17 Heavy Chain Variable Region
gaggtgcagctgcagcagtctggggcagaccttgtgaagccaggggcctcagtcaagttgtcctgcacaggttctggcttcaacattaaagacacctatatacactgggtgaggcagaggcctgaacagggcctggagtggattggaaggattgatcctgcgaatggtaaggctaattatgacccgaagttccagggcaaggccacaatgacagcagacacatcctccaagacagtctacgtgcagctcagcagcctgacatctgaggacactgccgtctattactgtgctcttgggggcgggtattatggtatggactattggggtcgaggaacctcagtcaccgtctcctca

SEQ ID NO: 80 - protein, 118 aa, Mus musculus
  misc_feature: SC37.17 Heavy Chain Variable Region
EVQLQQSGADLVKPGASVKLSCTGSGFNIKDTYIHWVRQRPEQGLEWIGRIDPANGKANYDPKFQGKATMTADTSSKTVYVQLSSLTSEDTAVYYCALGGGYYGMDYWGRGTSVTVSS

SEQ ID NO: 81 - DNA, 327 bp, Mus musculus
  misc_feature: SC37.19 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcgatatatttctgtgctctatggtccaacaaccattgggtgttcggtggaggaaccaaactgactgtccta

SEQ ID NO: 82 - protein, 109 aa, Mus musculus
  misc_feature: SC37.19 Light Chain Variable Region
QAVVTQESALTTSPGETVTLTCRSSTGAVTTSNYANWVQEKPDHLFTGLIGGTNNRAPGVPARFSGSLIGDKAALTITGAQTEDEAIYFCALWSNNHWVFGGGTKLTVL

SEQ ID NO: 83 - DNA, 351 bp, Mus musculus
  misc_feature: SC37.19 Heavy Chain Variable Region
cagatccagttggtgcagtctggacctgagctgaagaagcctggagagacagtcaagatctcctgcaaggcttctgggtatatcttcacaaactatggaatgaactgggtgaagcaggctccaggaaagggtctgaagtggatgggctggataaacacccacactggagagccaacatatagtgatgacttcaagggacggtttgccttgtctttggaaacctcagccagcactgcctttttgcagatcaacaacctcaaaaatgaggacacggctacatatttctgtgcaggatcctatgattactcgtttacttactggggccaagggactctggtcactgtctctgca

SEQ ID NO: 84 - protein, 117 aa, Mus musculus
  misc_feature: SC37.19 Heavy Chain Variable Region
QIQLVQSGPELKKPGETVKISCKASGYIFTNYGMNWVKQAPGKGLKWMGWINTHTGEPTYSDDFKGRFALSLETSASTAFLQINNLKNEDTATYFCAGSYDYSFTYWGQGTLVTVSA

SEQ ID NO: 85 - DNA, 327 bp, Mus musculus
  misc_feature: SC37.20 Light Chain Variable Region
caggctgttgtgacccaggaatctgcactcaccacatcacctggtgaaacagtcatactcacttgtcgttcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 86 - protein, 109 aa, Mus musculus
  misc_feature: SC37.20 Light Chain Variable Region
QAVVTQESALTTSPGETVILTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 87 - DNA, 351 bp, Mus musculus
  misc_feature: SC37.20 Heavy Chain Variable Region
cagatccagttggtgcagtctggacctgagctgaagaagcctggagagacagtcaagatctcctgcaaggcttctggttataccttcacaaactatggaatgaactgggtgaagcaggctccaggaaagggtttaaagtggatgggctggataaacaccgacactggagaggcaacatatggtgatgacttcaagggacggtttgccttttctttggaaacctctgccagcactgcctttttgcagatcaaaaacctcaaaaatgaggacacggctacatatttctgtgcaggatcctatgattattcgtttgcttactggggccaagggactctggtcactgtctctgaa

SEQ ID NO: 88 - protein, 117 aa, Mus musculus
  misc_feature: SC37.20 Heavy Chain Variable Region
QIQLVQSGPELKKPGETVKISCKASGYTFTNYGMNWVKQAPGKGLKWMGWINTDTGEATYGDDFKGRFAFSLETSASTAFLQIKNLKNEDTATYFCAGSYDYSFAYWGQGTLVTVSE

SEQ ID NO: 89 - DNA, 354 bp, Mus musculus
  misc_feature: SC37.21 Heavy Chain Variable Region
  misc_feature: SC37.21 Heavy Chain Variable Region
gaggttcggctgcagcagtctggggcagaccttgtgaagccaggggcctcagtcaagttgtcctgcacaggttctggcttcaacattaaagacacctatatacactgggtgaggcagaggcctgaacagggcctggagtggattggaaggattgatcctgcgaatggtaaggctaattatgacccgaagttccagggcaaggccacaatgacagcagacacatcctccaagacagtctacgtgcagctcagcagcctgacatctgaggacactgccgtctattactgtgctcttgggggcgggtattatggtatggactattggggtcgaggaacctcagtcaccgtctcctca

SEQ ID NO: 90 - protein, 118 aa, Mus musculus
  misc_feature: SC37.21 Heavy Chain Variable Region
EVRLQQSGADLVKPGASVKLSCTGSGFNIKDTYIHWVRQRPEQGLEWIGRIDPANGKANYDPKFQGKATMTADTSSKTVYVQLSSLTSEDTAVYYCALGGGYYGMDYWGRGTSVTVSS

SEQ ID NO: 91 - DNA, 327 bp, Mus musculus
  misc_feature: SC37.22 Light Chain Variable Region
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgcggcaatatatttctgtgctctatggtgcagcaaccattgggtgttcggtggaggaaccaaactgactgtccta

SEQ ID NO: 92 - protein, 109 aa, Mus musculus
  misc_feature: SC37.22 Light Chain Variable Region
QAVVTQESALTTSPGETVTLTCRSSTGAVTTSNYANWVQEKPDHLFTGLIGGTNNRAPGVPARFSGSLIGDKAALTITGAQTEDAAIYFCALWCSNHWVFGGGTKLTVL

SEQ ID NO: 93 - DNA, 351 bp, Mus musculus
  misc_feature: SC37.22 Heavy Chain Variable Region
cagatccagttggtgcagtctggacctgagctgaagaagcctggagagacagtcaagatctcctgcaaggctcctgggtataccttcagaaactatggattgaactgggtgaagcaggctccaggaaagggtttaaagtggatgggttggataaacactaacactggagagccaacatatactgatgacttcaagggacggtttgccttctctttggatacctctgccagcactgcctatttgcagatcaataacctcaaaaatgaggacatggcaacatatttctgtgcaagatctcatgatttctcctttgtttattggggccaagggactctggtcactgtctctgca

SEQ ID NO: 94 - protein, 117 aa, Mus musculus
  misc_feature: SC37.22 Heavy Chain Variable Region
QIQLVQSGPELKKPGETVKISCKAPGYTFRNYGLNWVKQAPGKGLKWMGWINTNTGEPTYTDDFKGRFAFSLDTSASTAYLQINNLKNEDMATYFCARSHDFSFVYWGQGTLVTVSA

SEQ ID NO: 95 - DNA, 321 bp, Mus musculus
  misc_feature: SC37.23 Light Chain Variable Region
gacatccagatgacacaatcttcatcctccttttctgtatctctaggagacagagtcaccattacttgcaaggcaagtgaggacatatataatcggttagcctggtttcagcagaaaccaggaaatgctcctaggctcttgatatctggtgcaaccagtttggaaactggggttccttcaagattcagtggcagtagatctggagaggattacactctcatcattaccagtcttcagactgaggatgttgctacttattactgtcaacagttttggactactcctccgacgttcggtggaggcaccaagctggaaatcaaa

SEQ ID NO: 96 - protein, 107 aa, Mus musculus
  misc_feature: SC37.23 Light Chain Variable Region
DIQMTQSSSSFSVSLGDRVTITCKASEDIYNRLAWFQQKPGNAPRLLISGATSLETGVPSRFSGSRSGEDYTLIITSLQTEDVATYYCQQFWTTPPTFGGGTKLEIK

SEQ ID NO: 97 - DNA, 342 bp, Mus musculus
  misc_feature: SC37.23 Heavy Chain Variable Region
caggttcagctgcagcagtctggaactgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagttcaagggcaaggccacattcactgtagatacatcctccaacacagcctacatgcaactcagcagcctgacatctgaggactctgccgtctattactgtgcaagaataatacgggacttctggggccaaggcaccactctcacagtctcctca

SEQ ID NO: 98 - protein, 114 aa, Mus musculus
  misc_feature: SC37.23 Heavy Chain Variable Region
QVQLQQSGTELMKPGASVKISCKATGYTFSSYWIEWVKQRPGHGLEWIGEILPGSGSTNYNEKFKGKATFTVDTSSNTAYMQLSSLTSEDSAVYYCARIIRDFWGQGTTLTVSS

SEQ ID NO: 99 - DNA, 342 bp, Mus musculus
  misc_feature: SC37.28 Heavy Chain Variable Region
caggttcagctgcagcagtctggaactgagctgatgaagcctggggcctcagtgaagatatcctgcaaggctactggctacacattcagtgcctattggatagagtgggtaaagcagaggcctggacatggccttgagtggattggagagattttacctggaagtggtagtactaactgcaatgagaagttcagggccaaggccacattcactgcagatacatcctccaacacagcctacatgcaactcagcagcctgacatctgaggactctgccgtctattactgtgcaagaatactacgggacctctggggccaaggcaccactctcacagtctcctca

SEQ ID NO: 100 - protein, 114 aa, Mus musculus
  misc_feature: SC37.28 Heavy Chain Variable Region
QVQLQQSGTELMKPGASVKISCKATGYTFSAYWIEWVKQRPGHGLEWIGEILPGSGSTNCNEKFRAKATFTADTSSNTAYMQLSSLTSEDSAVYYCARILRDLWGQGTTLTVSS

SEQ ID NO: 101 - DNA, 321 bp, Mus musculus
  misc_feature: SC37.29 Light Chain Variable Region
gatattgtgctaactcagtctccagccaccctgtctgtgcctccaggagatggagtcagtctttcctgcagggccagtcaaagtatttgcaactacctacactggtttcaacaaaaatcacatgagtctccaaggcttctcatcaagtgtgcttcccagtccatctctgggatcccctccaggctcagtggcagcggatcagggacagatttcactctcagtatcaacagtgtggagacagaagattttggaatgtatttctgtcaacagagtaacaactggccgtggacgctcggtggaggctccagggtggaaatcaaa

SEQ ID NO: 102 - protein, 108 aa, Mus musculus
  misc_feature: SC37.29 Light Chain Variable Region
DDIVLTQSPATLSVPPGDGVSLSCRASQSICNYLHWFQQKSHESPRLLIKCASQSISGIPSRLSGSGSGTDFTLSINSVETEDFGMYFCQQSNNWPWTLGGGSRVEIK

SEQ ID NO: 103 - DNA, 351 bp, Mus musculus
  misc_feature: SC37.29 Heavy Chain Variable Region
cagatccagttggtgcagtctggacctgagctgaagaagcctggagagacagtcaagatctcctgcaaggcttctgggtataccttcacaaactatggaatgaactgggtgaaacaggctccaggaaagggtttgaagtggatgggctgggtaaacacccacactggagagccaacatatggtgatgacttcaagggacggtttgccttctctttggaaacctctgccagcactgcctatttgcagatcaacagcctcaaaaatgaggacatggctacatatttctgtgcaggatcttatgattactcctttgcttactggggccaagggactctggtcactgtctctgca

SEQ ID NO: 104 - protein, 117 aa, Mus musculus
  misc_feature: SC37.29 Heavy Chain Variable Region
QIQLVQSGPELKKPGETVKISCKASGYTFTNYGMNWVKQAPGKGLKWMGWVNTHTGEPTYGDDFKGRFAFSLETSASTAYLQINSLKNEDMATYFCAGSYDYSFAYWGQGTLVTVSA

SEQ ID NO: 105 - DNA, 321 bp, Mus musculus
  misc_feature: SC37.37 Light Chain Variable Region
caaattgttctcacccagtctccagcaatcatgtctgcatctccaggggagaaggtcaccataacctgcagtgccagctcaagtgtaagttacatgcactggttccagcagaagccaggcacttctcccaaactctggatttatggcacatccaacctggcttctggagtccctgctcgcttcagtggcagtggatctgggacctcttactctctcacaatcagccgaatggaggctgaagatgctgccacttattactgccagcaaaggagtagttacccaccctacacgttcggaggggggaccaagctggaaataaaa

SEQ ID NO: 106 - protein, 107 aa, Mus musculus
  misc_feature: SC37.37 Light Chain Variable Region
QIVLTQSPAIMSASPGEKVTITCSASSSVSYMHWFQQKPGTSPKLWIYGTSNLASGVPARFSGSGSGTSYSLTISRMEAEDAATYYCQQRSSYPPYTFGGGTKLEIK

SEQ ID NO: 107 - DNA, 339 bp, Mus musculus
  misc_feature: SC37.37 Heavy Chain Variable Region
caggtccagctgcagcagtctgcagctgaactggcaagacctggggcctcagtgaagatgtcctgcgaggcttctggctacacctttacaagctacacgatacactggataaaacagaggcctggacagggtctggaatggattggatacattaatcctaataatggatatactgagtacaatcagaagttcaaggacaagaccacagtgactgtagacaaatcctccagcacagcccacatgcaactgagcagcctgacatctgaggactctgcggtctattactgtgcaaggggactctcttactggggccaagggactctggtcactgtctctgca

SEQ ID NO: 108 - protein, 113 aa, Mus musculus
  misc_feature: SC37.37 Heavy Chain Variable Region
QVQLQQSAAELARPGASVKMSCEASGYTFTSYTIHWIKQRPGQGLEWIGYINPNNGYTEYNQKFKDKTTVTVDKSSSTAHMQLSSLTSEDSAVYYCARGLSYWGQGTLVTVSA

SEQ ID NO: 109 - DNA, 321 bp, Mus musculus
  misc_feature: SC37.39 Light Chain Variable Region
gacattgtgatgacccagtctcaaaaattcatgtccacatcaggaggagacagggtcagcgtcacctgcaaggccagtcagaatgtgggtattaatgtagcctggtatcaacagaaaccagggcaatctcctaaagcactgatttactcggcatcctaccggtacagtggagtccctaatcgcttcacaggcagtggatctgggacagatttcactctcaccatcagcaatgtgcagtctgaagacttgggagactatttctgtcaccaatataaaacctatccgtacacgttcggaggggggaccaagctggaaataaaa

SEQ ID NO: 110 - protein, 107 aa, Mus musculus
  misc_feature: SC37.39 Light Chain Variable Region
DIVMTQSQKFMSTSGGDRVSVTCKASQNVGINVAWYQQKPGQSPKALIYSASYRYSGVPNRFTGSGSGTDFTLTISNVQSEDLGDYFCHQYKTYPYTFGGGTKLEIK

SEQ ID NO: 111 - DNA, 339 bp, Mus musculus
  misc_feature: SC37.39 Heavy Chain Variable Region
caggtccagctacagcagtctgcagctgaactggcaagacctggggcctcagtgaagatgtcctgcaaggcttctggctacacctttactacctacacgatacactgggtaaaacagaggcctggacagggtctgaaatggattggatacattaatcctcgcagtggatatactgagtacaatcagaagttccaggacaagaccacaatgacctcagacaactcctccagcacagcctatattcaactgagcagcctgacatctgaggattctgcggtctattactgtgcaagatcctacgaattctggggccaaggcaccactctcacagtctcctcg

SEQ ID NO: 112 - protein, 113 aa, Mus musculus
  misc_feature: SC37.39 Heavy Chain Variable Region
QVQLQQSAAELARPGASVKMSCKASGYTFTTYTIHWVKQRPGQGLKWIGYINPRSGYTEYNQKFQDKTTMTSDNSSSTAYIQLSSLTSEDSAVYYCARSYEFWGQGTTLTVSS

SEQ ID NO: 113 - DNA, 333 bp, Mus musculus
  misc_feature: SC37.40 Light Chain Variable Region
aacattgtactgacccaatctccagcttctttggctgtgtctctagggcagagggccaccatttcctgcagagcccgtgaaagtgttgatacttatggcaatagttttatacactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 114 - protein, 111 aa, Mus musculus
  misc_feature: SC37.40 Light Chain Variable Region
NIVLTQSPASLAVSLGQRATISCRARESVDTYGNSFIHWYQQKPGQPPKLLIYLASNLESGVPARFSGSGSRTDFTLTIDPVEADDAATYYCQQNNEDPLTFGAGTKLELK

SEQ ID NO: 115 - DNA, 354 bp, Mus musculus
  misc_feature: SC37.40 Heavy Chain Variable Region
gaggttcagctgcagcagtctggggcagaccttgtgaagccaggggcctcagtcaagttgtcctgcacaggttctggcttcaacattaaagacacctatatacactgggtgaggcagaggcctgaacagggcctggagtggattggaaggattgatcctgcgaatggtaaggctaattatgacccgaagttccagggcaaggccacaataacagcagacacatcctccaagacagcctacgtgcagctcagcagcctgacatctgaggacactgccgtctattactgtgctcttgggggcgggtactatggtatggactactggggtcgaggaacctcagtcaccgtctcctca

SEQ ID NO: 116 - protein, 118 aa, Mus musculus
  misc_feature: SC37.40 Heavy Chain Variable Region
EVQLQQSGADLVKPGASVKLSCTGSGFNIKDTYIHWVRQRPEQGLEWIGRIDPANGKANYDPKFQGKATITADTSSKTAYVQLSSLTSEDTAVYYCALGGGYYGMDYWGRGTSVTVSS

SEQ ID NO: 117 - DNA, 333 bp, Mus musculus
  misc_feature: SC37.41 Light Chain Variable Region
aacattgtgctgacccaatctccagcttctttggctgtgtctctagggcagagggccaccatatcctgcagagccagtgaaagtgttgatagttatggcaatagttttatgcactggtaccagcagaaaccaggtcagccacccaaactcctcatctatcttgcatccaacctagaatctggggtccctgccaggttcagtggcagtgggtctaggacagacttcaccctcaccattgatcctgtggaggctgatgatgttgcaacctattactgtcagcaaaataatgaggatccgtacacgttcggaggggggaccaagctggaaataaaa

SEQ ID NO: 118 - protein, 111 aa, Mus musculus
  misc_feature: SC37.41 Light Chain Variable Region
NIVLTQSPASLAVSLGQRATISCRASESVDSYGNSFMHWYQQKPGQPPKLLIYLASNLESGVPARFSGSGSRTDFTLTIDPVEADDVATYYCQQNNEDPYTFGGGTKLEIK

SEQ ID NO: 119 - DNA, 357 bp, Mus musculus
  misc_feature: SC37.41 Heavy Chain Variable Region
gaggttcagctgcagcagtctggggcagagtttgtgaagccaggggcctcagtcaagttgtcctgcacagcttctggcttcaacattaaagacacctatatgcactgggtgaagcagaggcctgaacagggcctggagtggattggaaggattgatcctgcgaatggtaattctaaatataacccgaagttccaggccaaggccactataacaacagacacatcctccaacacagcctacctgcagctcagcagcctgacatctgaggacactgccgtctatttctgtactcccccaactgggccctactatggtatggactactggggtcaaggaacctcagtcaccgtctcctca

SEQ ID NO: 120 - protein, 119 aa, Mus musculus
  misc_feature: SC37.41 Heavy Chain Variable Region
EVQLQQSGAEFVKPGASVKLSCTASGFNIKDTYMHWVKQRPEQGLEWIGRIDPANGNSKYNPKFQAKATITTDTSSNTAYLQLSSLTSEDTAVYFCTPPTGPYYGMDYWGQGTSVTVSS

SEQ ID NO: 121 - DNA, 321 bp, Mus musculus
  misc_feature: SC37.44 Light Chain Variable Region
gatattgtgctaactcagtctccagccaccctgtctgtgactccaggagatagagtcagtctttcctgcagggccagtcaaaatattagcaactacctacactggtttcaacaaaaatcacatgagtctccaaggcttcttattaactatgcttcccagtccatctctgggatcccctccaggttcagtggcagtggatcagggacagatttcactctcagttttaatagtgtggagactgaagattttggactgtatttctgtcaacagagtaacagctggccgtggacgttcggtggaggcaccaagctggaaatcaaa

SEQ ID NO: 122 - protein, 107 aa, Mus musculus
  misc_feature: SC37.44 Light Chain Variable Region
DIVLTQSPATLSVTPGDRVSLSCRASQNISNYLHWFQQKSHESPRLLINYASQSISGIPSRFSGSGSGTDFTLSFNSVETEDFGLYFCQQSNSWPWTFGGGTKLEIK

SEQ ID NO: 123 - DNA, 321 bp, Mus musculus
  misc_feature: SC37.45 Light Chain Variable Region
gacatccagatgacacaatcttcatcctccttttctgtatctctaggagacagagtcaccattacttgcaaggcaagtgaggacatatataatcgcttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttatcactgtcaacagaattggagtactcctccgacgttcggtggaggcaccaagctggaaatcaaa

SEQ ID NO: 124 - protein, 107 aa, Mus musculus
  misc_feature: SC37.45 Light Chain Variable Region
DIQMTQSSSSFSVSLGDRVTITCKASEDIYNRLAWYQQKPGNAPRLLISGATSLETGVPSRFSGSGSGKDYTLSITSLQTEDVATYHCQQNWSTPPTFGGGTKLEIK

SEQ ID NO: 125 - DNA, 339 bp, Mus musculus
  misc_feature: SC37.45 Heavy Chain Variable Region
caggttcagctgcaacagtctggagctgagctgatgaagcctggggcctcagtgaagatatcctgcaaggccactggctacacattcagtaggtactggatagagtgggtaaagcagaggcctggacatggccttgagtggattggagagattttacctggaagtggtagtactaactacaatgagaagttcaagggcaaggccacattcactgcagatacatcctccaacacagcctacatgcaactcaccagcctgacatctgaggactctgccgtctatttctgtgaaagacggggggcttattggggccaagggactctggtcactgtctctgca

SEQ ID NO: 126 - protein, 113 aa, Mus musculus
  misc_feature: SC37.45 Heavy Chain Variable Region
QVQLQQSGAELMKPGASVKISCKATGYTFSRYWIEWVKQRPGHGLEWIGEILPGSGSTNYNEKFKGKATFTADTSSNTAYMQLTSLTSEDSAVYFCERRGAYWGQGTLVTVSA

SEQ ID NO: 127 - DNA, 333 bp, Mus musculus
  misc_feature: SC37.47 Light Chain Variable Region
aacattgtgctgacccaatctccagcttctttggctgtgtctctagggcagagggccaccatatcctgcagagccagtgaaagtgttgatacttatggcaatagttttatacactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 128 - protein, 111 aa, Mus musculus
  misc_feature: SC37.47 Light Chain Variable Region
NIVLTQSPASLAVSLGQRATISCRASESVDTYGNSFIHWYQQKPGQPPKLLIYLASNLESGVPARFSGSGSRTDFTLTIDPVEADDAATYYCQQNNEDPLTFGAGTKLELK

SEQ ID NO: 129 - DNA, 354 bp, Mus musculus
  misc_feature: SC37.47 Heavy Chain Variable Region
gaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagctgacacatcctccaacacagcctacctgcagctcagcagcctgacatctgaggacactgccgtctattactgtgctaccggaggtggttactatgctatggactactggggtcaaggaacctcagtcaccgtctcctca

SEQ ID NO: 130 - protein, 118 aa, Mus musculus
  misc_feature: SC37.47 Heavy Chain Variable Region
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCATGGGYYAMDYWGQGTSVTVSS

SEQ ID NO: 131 - DNA, 321 bp, Mus musculus
  misc_feature: SC37.48 Light Chain Variable Region
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaagagattgttagatggggtcccatcaaggttcagtggcagtggatctgggcaagattattctctcaccatcagcagcctggagtatgaagatatgggaatttattattgtctacagtatgatgaatttccgtacacgttcggaggggggaccaagctggaaataaaa

SEQ ID NO: 132 - protein, 107 aa, Mus musculus
  misc_feature: SC37.48 Light Chain Variable Region
DIKMTQSPSSMYASLGERVTITCKASQDINSYLSWFQQKPGKSPKTLIYRAKRLLDGVPSRFSGSGSGQDYSLTISSLEYEDMGIYYCLQYDEFPYTFGGGTKLEIK

SEQ ID NO: 133 - DNA, 342 bp, Mus musculus
  misc_feature: SC37.48 Heavy Chain Variable Region
caggtccagctgcagcagtctggggctgaactggcaagacctggggcctcagtgaagatgtcctgcaaggcttctggctacaccttcactagttacacgatacactgggtgaaacagaggcctggacagggtctggaatggcttggctacattaatcctcgcagtaattatactaattacaatcagaggttcaaggacaaggccacattgactgcagacaaatcctccagcacagcctacatgcaactgagcagcctgacatctgaagactctgcagtctattactgtacaagatggggggaggacttctggggccaaggcaccactctcacagtctcctca

SEQ ID NO: 134 - protein, 114 aa, Mus musculus
  misc_feature: SC37.48 Heavy Chain Variable Region
QVQLQQSGAELARPGASVKMSCKASGYTFTSYTIHWVKQRPGQGLEWLGYINPRSNYTNYNQRFKDKATLTADKSSSTAYMQLSSLTSEDSAVYYCTRWGEDFWGQGTTLTVSS

SEQ ID NO: 135 - DNA, 321 bp, Mus musculus
  misc_feature: SC37.51 Light Chain Variable Region
gatattgtgctaactcagtctccagccaccctgtctgtgcctccaggagatggagtcagtcttacctgcagggccagtcaaagtatttgcaactaccttcactggtatcaacaaaaatcccatgagtctccaaggcttctcatcaagtatacttcccagtccatctctgggatcccctccaggttcagtggctgtggatcagggacagatttcactctcagtatcaacagtgtggagactgaagattttggaatgtatttctgtcaacagagtaacagctggccgtggacgttcggtggaggcaccaagctggaaatcaaa

SEQ ID NO: 136 - protein, 107 aa, Mus musculus
  misc_feature: SC37.51 Light Chain Variable Region
DIVLTQSPATLSVPPGDGVSLTCRASQSICNYLHWYQQKSHESPRLLIKYTSQSISGIPSRFSGCGSGTDFTLSINSVETEDFGMYFCQQSNSWPWTFGGGTKLEIK

SEQ ID NO: 137 - DNA, 351 bp, Mus musculus
  misc_feature: SC37.51 Heavy Chain Variable Region
cagatccagttggtgcagtctggacctgaactgaagaagcctggagagacagtcaagatctcctgcaaggcttctgggtataccttcacaaactatggaatgaactgggtgaagcaggctccaggaaagggtttaaagtggatgggctggataaacacccacactggagagccaacatatgctgatgacttcaagggacggtttgccttctctttggacacctctgccaacactgcctatttacagatcaacaacctcagaaatgaggacatggctacatatttctgtgcaggatcttatgattactcctttgcttactggggccaagggactctggtcactgtctctgca

SEQ ID NO: 138 - protein, 117 aa, Mus musculus
  misc_feature: SC37.51 Heavy Chain Variable Region
QIQLVQSGPELKKPGETVKISCKASGYTFTNYGMNWVKQAPGKGLKWMGWINTHTGEPTYADDFKGRFAFSLDTSANTAYLQINNLRNEDMATYFCAGSYDYSFAYWGQGTLVTVSA

SEQ ID NO: 139 - DNA, 327 bp, Mus musculus
  misc_feature: SC37.72 Light Chain Variable Region
caggctgttgtgactcaggaatctgcactcattacatcacctggtgaaacagtcacactcacttgtcgttcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggctcagactgaggatgaggcaatatatttctgtgctctatggtacagcaaccattgggtgttcggtggaggaaccaaactgactgtccta

SEQ ID NO: 140 - protein, 109 aa, Mus musculus
  misc_feature: SC37.72 Light Chain Variable Region
QAVVTQESALITSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 141 - DNA, 351 bp, Mus musculus
  misc_feature: SC37.72 Heavy Chain Variable Region
cagatccagttggtgcagtctggacctgagctgaagaagcctggagagacagtcaagatctcctgcaaggctcctggggataccttcagaaactatggattgaactgggtgaagcaggctccaggaaagggtttaaagtggatgggctggataaacactaacactggagagccaacatatgatgatgacttcaagggacggtttgccttctctttggaaacctctgccaccacagcctatttgcagatcaataacttcaaaaatgaggacatggcaacatatttctgtgcaagatctcatgatttctcctttgtctattggggccaagggactctggtcactgtctctgca

SEQ ID NO: 142 - protein, 117 aa, Mus musculus
  misc_feature: SC37.72 Heavy Chain Variable Region
QIQLVQSGPELKKPGETVKISCKAPGDTFRNYGLNWVKQAPGKGLKWMGWINTNTGEPTYDDDFKGRFAFSLETSATTAYLQINNFKNEDMATYFCARSHDFSFVYWGQGTLVTVSA

SEQ ID NO: 143 - DNA, 333 bp, Mus musculus
  misc_feature: SC37.75 Light Chain Variable Region
aacattgtgctgacccaatctccagcttctttggctgtgtctctagggcagagggccaccatatcctgcagagccagtgaaagtgttgatacttatggcaatagttttatacactggtaccggcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 144 - protein, 111 aa, Mus musculus
  misc_feature: SC37.75 Light Chain Variable Region
NIVLTQSPASLAVSLGQRATISCRASESVDTYGNSFIHWYRQKPGQPPKLLIYLASNLESGVPARFSGSGSRTDFTLTIDPVEADDAATYYCQQNNEDPLTFGAGTKLELK

SEQ ID NO: 145 - DNA, 321 bp, Mus musculus
  misc_feature: SC37.77 Light Chain Variable Region
gacatcaagatgacccagtctccatcttccatgtatgcatctcttggagagagagtcactatcacttgcaaggcgagtcaggacattaatagctatttaagctggttccagcagaaaccagggaattctcctaagaccctgatctatcgtgcaaacagattgttagatggggtcccatcaaggttcagtggcagtggatctgggcaagattattctctcactatcagcagcctggactatgaagatatgggaatttattattgtctacagtatgatgagtttccgtacacgttcggaggggggaccaagctggaaataaaa

SEQ ID NO: 146 - protein, 107 aa, Mus musculus
  misc_feature: SC37.77 Light Chain Variable Region
DIKMTQSPSSMYASLGERVTITCKASQDINSYLSWFQQKPGNSPKTLIYRANRLLDGVPSRFSGSGSGQDYSLTISSLDYEDMGIYYCLQYDEFPYTFGGGTKLEIK

SEQ ID NO: 147 - DNA, 341 bp, Mus musculus
  misc_feature: SC37.77 Heavy Chain Variable Region
aggtccagctgcagcagtctggggctgaactggcaagacctggggcctcagtgaagatgtcctgcaaggcttctggctacacctttactagctacacgatacactgggtaaaacagaggcctggacagggtctggaatggattggatacattaatcctagaaataattatactaattacaatcagaacttcaaggacaaggccacattgactgtagacaaatcctccagcacagcctacatgcaactgagcagcctgacatctgaggactctgcagtctattactgtgcaagatggggggaggactactggggccaaggctccactctcacagtctcctca

SEQ ID NO: 148 - protein, 114 aa, Mus musculus
  misc_feature: SC37.77 Heavy Chain Variable Region
QVQLQQSGAELARPGASVKMSCKASGYTFTSYTIHWVKQRPGQGLEWIGYINPRNNYTNYNQNFKDKATLTVDKSSSTAYMQLSSLTSEDSAVYYCARWGEDYWGQGSTLTVSS

SEQ ID NO: 149 - DNA, 336 bp, Mus musculus
  misc_feature: SC37.108 Light Chain Variable Region
gatgttgtgatgacccagactccactcactttgtcggttaccattggacaaccagcctccatctcttgcaagtcaagtcagagcctcctagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattctccgtggacgttcggtggaggcaccaagctggaaatcaaa

SEQ ID NO: 150 - protein, 112 aa, Mus musculus
  misc_feature: SC37.108 Light Chain Variable Region
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHSPWTFGGGTKLEIK

SEQ ID NO: 151 - DNA, 339 bp, Mus musculus
  misc_feature: SC37.108 Heavy Chain Variable Region
caggtccaactgcagcagcctggggctgaactggtgaagcctggggcttcagtgaagttgtcctgcaaggcttctggctacaccttcaccagctactatatatactgggtgaagcagaggcctggacaaggccttgactggattggggggattaatcctaggaatggtggtactaacttcaatgagaagttcaagagcaaggccacactgactgtagacaaatcctccagcacagcctacatgcaactcagcagcctgacatctgaggactctgcggtctattactgtacaagatcgtggacttactggggccaagggactctggtcactgtctctgca

SEQ ID NO: 152 - protein, 113 aa, Mus musculus
  misc_feature: SC37.108 Heavy Chain Variable Region
QVQLQQPGAELVKPGASVKLSCKASGYTFTSYYIYWVKQRPGQGLDWIGGINPRNGGTNFNEKFKSKATLTVDKSSSTAYMQLSSLTSEDSAVYYCTRSWTYWGQGTLVTVSA

SEQ ID NO: 153 - DNA, 336 bp, Mus musculus
  misc_feature: SC37.122 Light Chain Variable Region
gatgttttgatgacccaaactccactctccctgcctgtcagtcttggagatcaagcctccatctcttgcagatctagtcagaccattgtacatagtgatggcaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 154 - protein, 112 aa, Mus musculus
  misc_feature: SC37.122 Light Chain Variable Region
DVLMTQTPLSLPVSLGDQASISCRSSQTIVHSDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 155 - DNA, 342 bp, Mus musculus
  misc_feature: SC37.122 Heavy Chain Variable Region
caggttcaactgcagcagtctggagctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtactgcctacaatcagaagttcaagggcaaggccacactgactgcagacaaatcctccagcacagcctacatggagctcagcagcctgacatctgaggactctgccgtctattactgtacccggacggggtttgcttactggggccaagggactctggtcactgtctctgca

SEQ ID NO: 156 - protein, 114 aa, Mus musculus
  misc_feature: SC37.122 Heavy Chain Variable Region
QVQLQQSGAELVRPGASVTLSCKASGYTFTDYEMHWVKQTPVHGLEWIGAIDPETGGTAYNQKFKGKATLTADKSSSTAYMELSSLTSEDSAVYYCTRTGFAYWGQGTLVTVSA

SEQ ID NO: 157 - DNA, 321 bp, Mus musculus
  misc_feature: SC37.127 Light Chain Variable Region
gacatccagatgactcagtctccagcctccctatctgtatctgtgggagaaactgtcaccatcacatgtcgagcaagtgagaatatttacagtaaattaggatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtacacgttcggaggggggaccaagctggaaaaaaaa

SEQ ID NO: 158 - protein, 107 aa, Mus musculus
  misc_feature: SC37.127 Light Chain Variable Region
DIQMTQSPASLSVSVGETVTITCRASENIYSKLGWYQQKQGKSPQLLVYAATNLADGVPSRFSGSGSGTQYSLKINSLQSEDFGSYYCQHFWGTPYTFGGGTKLEKK

SEQ ID NO: 159 - DNA, 336 bp, Mus musculus
  misc_feature: SC37.136  Light Chain Variable Region
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagttcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggaggggggaccaagctggaaataaaa

SEQ ID NO: 160 - protein, 112 aa, Mus musculus
  misc_feature: SC37.136 Light Chain Variable Region
DIVMTQAAPSVPVTPGESVSISCRSSKSLLHSNGNTYLYWFLQRPGQSPQFLIYRMSNLASGVPDRFSGSGSGTAFTLRISRVEAEDVGVYYCMQHLEYPFTFGGGTKLEIK

SEQ ID NO: 161 - DNA, 351 bp, Mus musculus
  misc_feature: SC37.136 Heavy Chain Variable Region
gaggttcagctgcagcagtctggggctgagcttgtgaggccaggggcctcagtcaagttgtcctgcacagcttctggctttaacattgaggccgactatatgcactgggtgaagcagaggcctgaacagggcctggagtggattggatggattgatcctgagattggttctactgaatatgcctcgaagttccagggcaaggccactataacagcagacacatcctccaacacagcctacctgcagctcagcagcctgacatctgaggacactgccgtctattactgtattacagatggtaactacgtgaagggctactggggccaaggcaccactctcacagtctcctca

SEQ ID NO: 162 - protein, 117 aa, Mus musculus
  misc_feature: SC37.136 Heavy Chain Variable Region
EVQLQQSGAELVRPGASVKLSCTASGFNIEADYMHWVKQRPEQGLEWIGWIDPEIGSTEYASKFQGKATITADTSSNTAYLQLSSLTSEDTAVYYCITDGNYVKGYWGQGTTLTVSS

SEQ ID NO: 163 - DNA, 321 bp, Mus musculus
  misc_feature: SC37.141 Light Chain Variable Region
gatatccagatgacacagagtacatcttccctgtctgcctctctgggagacagagtcaccatcagttgcagggcaagtcaggacattagcaattatttaaactggtatcagcagaaaccagatggaactgttaaaccccttatctattacacatcaagattacactcaggagtcccatcaaggttcagtggcagtgggtctggaacagattattctctcaccattagcaacctggaccaagaagatattgccacttacttttgccaacagggtaatacgcttccattcacgttcggctcggggacaaagttggcaatagag

SEQ ID NO: 164 - protein, 107 aa, Mus musculus
  misc_feature: SC37.141 Light Chain Variable Region
DIQMTQSTSSLSASLGDRVTISCRASQDISNYLNWYQQKPDGTVKPLIYYTSRLHSGVPSRFSGSGSGTDYSLTISNLDQEDIATYFCQQGNTLPFTFGSGTKLAIE

SEQ ID NO: 165 - DNA, 366 bp, Mus musculus
  misc_feature: SC37.141 Heavy Chain Variable Region
caggtccaactgcagcagcctggggctgagctggtgaggcctggggcttcagtgaagctgtcctgcaaggcttctggctacaccttcaccagctactggatgaactgggtgaaacagagacctggacaaggccttgaatggattggtatgattgatccttcagacagtgaaactcactacagtcaaatgttcaaggacaaggccacattgactgtagaccaatcctccaacacagcctacatgcacctcagcagcctgacatctgaggactctgcggtctattactgtgcaagagaggggtattacttcgatggtaccagggggattgcttactggggccaagggactctggtcactgtctctgta

SEQ ID NO: 166 - protein, 122 aa, Mus musculus
  misc_feature: SC37.141 Heavy Chain Variable Region
QVQLQQPGAELVRPGASVKLSCKASGYTFTSYWMNWVKQRPGQGLEWIGMIDPSDSETHYSQMFKDKATLTVDQSSNTAYMHLSSLTSEDSAVYYCAREGYYFDGTRGIAYWGQGTLVTVSV

SEQ ID NO: 167 - DNA, 336 bp, Mus musculus
  misc_feature: SC37.150 Light Chain Variable Region
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagggtggaggctgaggatctgggagtttatttctgctctcaaagtacacatgttccgctcacattcggtgctgggaccaagctggagctgaaa

SEQ ID NO: 168 - protein, 112 aa, Mus musculus
  misc_feature: SC37.150 Light Chain Variable Region
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 169 - DNA, 369 bp, Mus musculus
  misc_feature: SC37.150  Heavy Chain Variable Region
cagatccagttggtgcagtctggacctgagctgaagaagcctggagagacagtcaagatctcctgcaaggcttctgggtataccttcacaaactatccaatacactgggtgaagcaggctccaggaaagggtttaaagtggatgggctggataaacacctactctggagtgccaacatatgcagatgacttcaagggacggtttgccttctctttggaaacctctgccagcactgcatatttgcagatcaataacctcaaaaatgaggacatggctacatatttctgtgaagggggcggtatctactataataactacgggtatgttatggactactggggtcaaggaacctcagtcaccgtctcctca

SEQ ID NO: 170 - protein, 123 aa, Mus musculus
  misc_feature: SC37.150 Heavy Chain Variable Region
QIQLVQSGPELKKPGETVKISCKASGYTFTNYPIHWVKQAPGKGLKWMGWINTYSGVPTYADDFKGRFAFSLETSASTAYLQINNLKNEDMATYFCEGGGIYYNNYGYVMDYWGQGTSVTVSS

SEQ ID NO: 171 - DNA, 318 bp, Mus musculus
  misc_feature: SC37.160 Light Chain Variable Region
caaattgttctcacccagtctccagcaatcatgtctgcatctccaggggagaaggtctccataacctgcagtgccagctcaagtgtaagttacatgcactggttccagcagaagccgggcacttctcccaaactctggatttatagcacatccaacctggcttctggagtccctactcgcttcagtggcagtggatctgggacctcttactctctcacaatcagccgaatggaggctgaagatgctgccacttattactgccagcaaaggagtaattacccattcacgttcggctcggggacaaagttggaaataaaa

SEQ ID NO: 172 - protein, 106 aa, Mus musculus
  misc_feature: SC37.160 Light Chain Variable Region
QIVLTQSPAIMSASPGEKVSITCSASSSVSYMHWFQQKPGTSPKLWIYSTSNLASGVPTRFSGSGSGTSYSLTISRMEAEDAATYYCQQRSNYPFTFGSGTKLEIK

SEQ ID NO: 173 - DNA, 360 bp, Mus musculus
  misc_feature: SC37.160  Heavy Chain Variable Region
caggttcagctgcagcggtctggagctgagctggcgaggcctggggcttcagtgaagctgtcctgcaaggcttctggctacaccttcacaagctatggtataagctgggtgaagcagagaactggacagggccttgagtggattggagagatttttcctggaagtggtaatacttactacaatgagatgttcaagggcaaggccacactgactgcagacaaatcctccagcacagcgtacatggagctccgcagcctgacatctgaggactctgcggtctatttctgtgcaagaggggattactacggtagtagctcctttgactactggggccaaggcaccactctcacagtctcctca

SEQ ID NO: 174 - protein, 120 aa, Mus musculus
  misc_feature: SC37.160 Heavy Chain Variable Region
QVQLQRSGAELARPGASVKLSCKASGYTFTSYGISWVKQRTGQGLEWIGEIFPGSGNTYYNEMFKGKATLTADKSSSTAYMELRSLTSEDSAVYFCARGDYYGSSSFDYWGQGTTLTVSS

SEQ ID NO: 175 - DNA, 336 bp, Mus musculus
  misc_feature: SC37.163  Light Chain Variable Region
gatgttttgatgacccaaactccactctccctgcctgtcagtcttggagatcaagcctccatctcttgcagatctagtcagagcattgtacatagtaatggaaacacctatttacaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 176 - protein, 112 aa, Mus musculus
  misc_feature: SC37.163 Light Chain Variable Region
DVLMTQTPLSLPVSLGDQASISCRSSQSIVHSNGNTYLQWYLQKPGQSPKLLIYKVSNRFSGVPDRFSGSGSGTDFTLKISRVEAEDLGVYYCFQGSHVPPTFGGGTKLEIK

SEQ ID NO: 177 - DNA, 342 bp, Mus musculus
  misc_feature: SC37.163  Heavy Chain Variable Region
caggttcaactgcagcagtctggagctgagctggtgaggcctggggcttcagtgacgctgtcctgcaaggcttcgggctacacatttactgactatgaaatgcactggctgaagcagacacctgtgcatggcctggaatggattggagctattgatcctgaaactggtggtactgcctacaatcagaagttcaagggcaaggccacactgactgcagacaaatcctccagcacagcctccatggagctcagcagcctgacatctgaggactctgccgtctattactgtacccggacggggtttgtttactggggccaagggactctaatcactgtctctgca

SEQ ID NO: 178 - protein, 114 aa, Mus musculus
  misc_feature: SC37.163 Heavy Chain Variable Region
QVQLQQSGAELVRPGASVTLSCKASGYTFTDYEMHWLKQTPVHGLEWIGAIDPETGGTAYNQKFKGKATLTADKSSSTASMELSSLTSEDSAVYYCTRTGFVYWGQGTLITVSA

SEQ ID NO: 179 - DNA, 321 bp, Mus musculus
  misc_feature: SC37.169  Light Chain Variable Region
gacattgtgatgacccagtctcaaaaattcatgtccacatcagtaggagacagggtcagcgtcacctgcaaggccagtcagaatgtgggtactaatgtcgcctggtttcaacagaaaccagggcaatctcctaaactactgatttactcggcatcctaccgatacagtggagtccctgatcgcttcacaggcagtggatctgggacagatttcactctcaccatcagcaatgtgcagtctgaagacttggcagagtttttctgtcagcaatataacacctatcccctcacgttcggtgctgggaccaagctggagctgaaa

SEQ ID NO: 180 - protein, 107 aa, Mus musculus
  misc_feature: SC37.169 Light Chain Variable Region
DIVMTQSQKFMSTSVGDRVSVTCKASQNVGTNVAWFQQKPGQSPKLLIYSASYRYSGVPDRFTGSGSGTDFTLTISNVQSEDLAEFFCQQYNTYPLTFGAGTKLELK

SEQ ID NO: 181 - DNA, 348 bp, Mus musculus
  misc_feature: SC37.169  Heavy Chain Variable Region
caggttcagctgcagcagtctggagctgaggtgatgaagcctggggcctcagtgaagctttcctgcaaggctactggctacacattcactggctactggatagagtgggtaaaggagaggcctggacatggccttgagtggattggagagattttacctggaagtggtagtactaactacaatgagaagttcaagggcaaggccacattcactgcagatccatcctccaatacagcctacatgcaactcagcagcctgacaactgaggactctgccatctattactgtgcaagggactacggctcctttggttactggggccaagggactctggtcactgtctctgca

SEQ ID NO: 182 - protein, 116 aa, Mus musculus
  misc_feature: SC37.169 Heavy Chain Variable Region
QVQLQQSGAEVMKPGASVKLSCKATGYTFTGYWIEWVKERPGHGLEWIGEILPGSGSTNYNEKFKGKATFTADPSSNTAYMQLSSLTTEDSAIYYCARDYGSFGYWGQGTLVTVSA

SEQ ID NO: 183 - DNA, 321 bp, Mus musculus
  misc_feature: SC37.170 Light Chain Variable Region
gacattgtgatgacccagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcactggatctgggacggatttcactttcaccatcagcagtgtgcaggctgaagacctggcagtttattactgtcagcaacattatagtactccgctcacgttcggtgctgggaccaagctggagctgaaa

SEQ ID NO: 184 - protein, 107 aa, Mus musculus
  misc_feature: SC37.170 Light Chain Variable Region
DIVMTQSHKFMSTSVGDRVSITCKASQDVSTAVAWYQQKPGQSPKLLIYSASYRYTGVPDRFTGTGSGTDFTFTISSVQAEDLAVYYCQQHYSTPLTFGAGTKLELK

SEQ ID NO: 185 - DNA, 348 bp, Mus musculus
  misc_feature: SC37.170 Heavy Chain Variable Region
caggtccaactgcagcagcctgggtctatgctggtgaggcctggagcttcagtgaagctgtcctgcaaggcttctggctacacattcacccactactggatgcactgggtgaagcagaggcctggtcaaggccttgagtggattggagagatttatcctaatagtggtaggactaactacaatgagaagttcaagggcaaggccacactgactgttgacacatcctccagcacagcctacgtggatcttagtagcctgacatctgcggactctgcggtctattactgtgcaagaggatatgatccctttgactactggggccaaggcaccactctcacagtctcctca

SEQ ID NO: 186 - protein, 116 aa, Mus musculus
  misc_feature: SC37.170 Heavy Chain Variable Region
QVQLQQPGSMLVRPGASVKLSCKASGYTFTHYWMHWVKQRPGQGLEWIGEIYPNSGRTNYNEKFKGKATLTVDTSSSTAYVDLSSLTSADSAVYYCARGYDPFDYWGQGTTLTVSS

SEQ ID NO: 187 - DNA, 351 bp, Mus musculus
  misc_feature: SC37.172 Heavy Chain Variable Region
gaggttcagctgcagcagtctggggctgagcttgtgaggccaggggcctcagtcaagttgtcctgcacagcttctggctttaacattgaggccgactatatgcactgggtgaagcagagccctgaacagggcctggagtggattggatggattgatcctgagattggtgctactgaatatgcctcgaagttccagggcaaggccactatgacagcagacacatcctccaacacagcctacctgcagctcagcagcctgacatctgaggacactgccgtctattactgtattacagatggtaactacgtgaagggctactggggccaaggcaccactctcacagtctcctca

SEQ ID NO: 188 - protein, 117 aa, Mus musculus
  misc_feature: SC34.95 Heavy Chain Variable Region
EVQLQQSGAELVRPGASVKLSCTASGFNIEADYMHWVKQSPEQGLEWIGWIDPEIGATEYASKFQGKATMTADTSSNTAYLQLSSLTSEDTAVYYCITDGNYVKGYWGQGTTLTVSS

SEQ ID NO: 189 - DNA, 336 bp, Mus musculus
  misc_feature: SC37.177 Light Chain Variable Region
gacattgtgatgtcacagtctccatcctccctagctgtgtcagttggagagaaggttactatgagctgcaagtccagtcagagccttttatatagtaccaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatgactattggacgttcggtggaggcaccaagctggaaatcaaa

SEQ ID NO: 190 - protein, 112 aa, Mus musculus
  misc_feature: SC37.177 Light Chain Variable Region
DIVMSQSPSSLAVSVGEKVTMSCKSSQSLLYSTNQKNYLAWYQQKPGQSPKLLIYWASTRESGVPDRFTGSGSGTDFTLTISSVKAEDLAVYYCQQYYDYWTFGGGTKLEIK

SEQ ID NO: 191 - DNA, 366 bp, Mus musculus
  misc_feature: SC37.177  Heavy Chain Variable Region
caggtttctctgatagagtcgggccctgggataatgcagccctcccagaccctcagtctgacttgctctttctctgggttttcactgagcgcttctgatatgggtgtaggctggattcgtcagccttcagggaagggtctggaatggctggcaaacatttggtgggatgatagtaagtactataacgccgccctgaagacccggctcacaatctccaaggatacctccaaaaaccaggtattcctcaagatcgccagtgtggacactgcagatactgccacatactactgtgctcgaatatctagggcctactatggtcactggtttgcttactggggccaagggactctggtcactgtctctgca

SEQ ID NO: 192 - protein, 122 aa, Mus musculus
  misc_feature: SC37.177 Heavy Chain Variable Region
QVSLIESGPGIMQPSQTLSLTCSFSGFSLSASDMGVGWIRQPSGKGLEWLANIWWDDSKYYNAALKTRLTISKDTSKNQVFLKIASVDTADTATYYCARISRAYYGHWFAYWGQGTLVTVSA

SEQ ID NO: 193 - DNA, 336 bp, Mus musculus
  misc_feature: SC37.185  Light Chain Variable Region
gacattgtgatgtcacagtctccatcctccctagctgtgtcagttggagagaaggttactatgagctgcaagtccagtcagagccttttatatagtagaaatcaaaagaactacttggcctggtaccagcagaaaccagggcagtctcctaaactgctgatttattgggcatccactagggaatctggggtccctgatcgcttcacaggcagtggatctgggacagatttcactctcaccatcagcagtgtgaaggctgaagacctggcagtttattactgtcagcaatattatagctattggacgttcggtggaggctccaagctggaaatcaac

SEQ ID NO: 194 - protein, 112 aa, Mus musculus
  misc_feature: SC37.185 Light Chain Variable Region
DIVMSQSPSSLAVSVGEKVTMSCKSSQSLLYSRNQKNYLAWYQQKPGQSPKLLIYWASTRESGVPDRFTGSGSGTDFTLTISSVKAEDLAVYYCQQYYSYWTFGGGSKLEIN

SEQ ID NO: 195 - DNA, 363 bp, Mus musculus
  misc_feature: SC37.185  Heavy Chain Variable Region
caggtccaactgcagcagccgggggctgagctggtgaggcctgggtcttcagtgaagctgtcctgcaaggcttctggctacaccttcaccagctattggatggattgggtgaagcagaggcctggacaaggccttgaatggattggtaacatttacccttctgatggtgaaactcactacaatcaaaagttcaaggacaaggccacattgaccgtagacagattgtccagcacagcctacatgcagctcagcagcctgacatctgaggactctgcggtctattactgtgcaagacattactacgctggtagcccgtactactttgactactggggccaaggcaccactctcacagtctcctca

SEQ ID NO: 196 - protein, 121 aa, Mus musculus
  misc_feature: SC37.185 Heavy Chain Variable Region
QVQLQQPGAELVRPGSSVKLSCKASGYTFTSYWMDWVKQRPGQGLEWIGNIYPSDGETHYNQKFKDKATLTVDRLSSTAYMQLSSLTSEDSAVYYCARHYYAGSPYYFDYWGQGTTLTVSS

SEQ ID NO: 197 - DNA, 321 bp, Mus musculus
  misc_feature: SC37.191 Light Chain Variable Region
aggattgtgatgacccagactcccaaatccctgcttgtatcagcaggagacagggtgaccataacctgcaaggccagtcagagtgtgagtaatgatgtaacttggtaccaacagaagccagggcagtctcctaaactgctcatatactatgcatccaatcgcaacactggagtacctgatcgcttcactggcagtggatatgggacggatttcactttcaccatcagcactgtgcaggctgaagacctggcagtttatttctgtcagcaggattatggctctccattcacgttcgggtcggggacaaagttggagataaaa

SEQ ID NO: 198 - protein, 107 aa, Mus musculus
  misc_feature: SC37.191 Light Chain Variable Region
RIVMTQTPKSLLVSAGDRVTITCKASQSVSNDVTWYQQKPGQSPKLLIYYASNRNTGVPDRFTGSGYGTDFTFTISTVQAEDLAVYFCQQDYGSPFTFGSGTKLEIK

SEQ ID NO: 199 - DNA, 345 bp, Mus musculus
  misc_feature: SC37.191 Heavy Chain Variable Region
caggtccaactgcagcagcctgggtctgtgctggtgaggcctggagcttcagtgaagctgtcctgcaaggcttctggctacacattcaccaactactggttgcactgggtgaagcagaggcctggacaaggccttgagtggattggagagatttatcctaatagtggtaggactaactacaatgagaagttcaaggacaaggccacactgactggagacacatcctccaacacagcctacgtggatctcatcagcctgacatctgaggactctgcggtctattactgtgcggtagctaccccacctgactactggggccaaggcaccactctcacagtctcctca

SEQ ID NO: 200 - protein, 115 aa, Mus musculus
  misc_feature: SC37.191 Heavy Chain Variable Region
QVQLQQPGSVLVRPGASVKLSCKASGYTFTNYWLHWVKQRPGQGLEWIGEIYPNSGRTNYNEKFKDKATLTGDTSSNTAYVDLISLTSEDSAVYYCAVATPPDYWGQGTTLTVSS

SEQ ID NO: 201 - DNA, 336 bp, Mus musculus
  misc_feature: SC37.193  Light Chain Variable Region
gatattgtgatgactcaggatgcaccctctatacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggctcggggacaaagttggaaataaaa

SEQ ID NO: 202 - protein, 112 aa, Mus musculus
  misc_feature: SC37.193 Light Chain Variable Region
DIVMTQDAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 203 - DNA, 348 bp, Mus musculus
  misc_feature: SC37.193  Heavy Chain Variable Region
gaggttcagttgcagcagtctggggctgaacttgtgaggccaggggcctcagtcaagttgtcctgcacagcttctggctttaacattaaggacgactatatgcactggatgaagcagaggcctgaacagggcctggaatggcttggatggattgatcctgagattggtgctactgaatatgcctcgaagttccagggcaaggccactatgacagcggacacatcctccaacacagcctacctgcagctcagcagcctgacatctgaggacactgccgtctattactgtgtcgatgaccgtcgtggtatggactactggggtcaaggaacctcagtcaccgtctcctca

SEQ ID NO: 204 - protein, 116 aa, Mus musculus
  misc_feature: SC37.193 Heavy Chain Variable Region
EVQLQQSGAELVRPGASVKLSCTASGFNIKDDYMHWMKQRPEQGLEWLGWIDPEIGATEYASKFQGKATMTADTSSNTAYLQLSSLTSEDTAVYYCVDDRRGMDYWGQGTSVTVSS

SEQ ID NO: 205 - DNA, 336 bp, Mus musculus
  misc_feature: SC37.196  Light Chain Variable Region
gatattgtgatgactcaggctgccccctctgtacctgtcactcctggagagtcagtatccatctcctgcaggtctagtaagagtctcctacatagtaatggcaacacttacttatattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggctcggggacaaagttggaaataaaa

SEQ ID NO: 206 - protein, 112 aa, Mus musculus
  misc_feature: SC37.196 Light Chain Variable Region
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 207 - DNA, 345 bp, Mus musculus
  misc_feature: SC37.196  Heavy Chain Variable Region
gaggttcagctgcagcagtctgggactgagcttgtgaggccaggggcctcagtcaagttgtcctgcacagcttctggctttaacattgaagccgactatttgcactgggtgaagcagaggcctgaacagggcctggagtggattggctggattgatcctgaaattggttctactgaatatgcctcgaaattccggggcaaggcctctataacagcagacacatcctccaacacagcctacctgcagctcaacagcctgacatctgaggacactgccgtctattactgtgctattgacgggactatggactactggggtcaaggaacctcagtcaccgtctcctca

SEQ ID NO: 208 - protein, 115 aa, Mus musculus
  misc_feature: SC37.196 Heavy Chain Variable Region
EVQLQQSGTELVRPGASVKLSCTASGFNIEADYLHWVKQRPEQGLEWIGWIDPEIGSTEYASKFRGKASITADTSSNTAYLQLNSLTSEDTAVYYCAIDGTMDYWGQGTSVTVSS

SEQ ID NO: 209 - DNA, 336 bp, Mus musculus
  misc_feature: SC37.202  Light Chain Variable Region
gacattgtgatgtcacagtctccatcctccctagctgtgtcagttggagaggaagttactatgagctgtaagtccagtcagagccttttatatagtaccaatcaaaagaactacttggcctggtaccagcagaagccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatgactattacacgttcggaggggggaccaagctggaaataaaa

SEQ ID NO: 210 - protein, 112 aa, Mus musculus
  misc_feature: SC37.202 Light Chain Variable Region
DIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCQQYYDYYTFGGGTKLEIK

SEQ ID NO: 211 - DNA, 357 bp, Mus musculus
  misc_feature: SC37.202  Heavy Chain Variable Region
caggtttctctgaaagagtcgggccctgggatattgcagccctcccagaccctcagtctgacttgttctttctctgggttttcactgagcacttctggtatggctgtaggctggattcgtcagccttcagggaggggtctggagtggctggcaaacatttggtgggatgatagtcagcattataacgcagccctgaagagccggctcacaatctccaaggatacctccaaaaaccaggtattcctcaagatcgccagtgtggacactgcagatactgccacatactactgtgctcgttctaactggggccgttactttgactactggggccaaggcaccactctcacagtctcctca

SEQ ID NO: 212 - protein, 119 aa, Mus musculus
  misc_feature: SC37.202 Heavy Chain Variable Region
QVSLKESGPGILQPSQTLSLTCSFSGFSLSTSGMAVGWIRQPSGRGLEWLANIWWDDSQHYNAALKSRLTISKDTSKNQVFLKIASVDTADTATYYCARSNWGRYFDYWGQGTTLTVSS

SEQ ID NO: 213 - DNA, 336 bp, Mus musculus
  misc_feature: SC37.212 Light Chain Variable Region
gatattgtgatgactcaggctgtaccctctgtacctgtcactcctggagagtcagtatccatctcctgcaggtctagtaagagtctcctgcatagtaatggcaacacttacttgtattggttcctgcagaggccaggccagtctcctcagttcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagagtatcctttcacgttcggaggggggaccaggctggaaataaaa

SEQ ID NO: 214 - protein, 112 aa, Mus musculus
  misc_feature: SC37.212 Light Chain Variable Region
DIVMTQAVPSVPVTPGESVSISCRSSKSLLHSNGNTYLYWFLQRPGQSPQFLIYRMSNLASGVPDRFSGSGSGTAFTLRISRVEAEDVGVYYCMQHLEYPFTFGGGTRLEIK

SEQ ID NO: 215 - DNA, 351 bp, Mus musculus
  misc_feature: SC37.212  Heavy Chain Variable Region
gaggttcagctgcagcagtctggggctgagcttgtgaggccaggggcctcagtcaagttgtcctgcacagcttctggctttaacattgaagttgactatatgcactgggtgaagcagaggcctgaacagggcctggagtggattggatggattgatcctgagattggtggtactgaatatgcctcgaagttccagggcaaggccactataacagcagacacatcctccaacacagtctacctgcacctcagcagcctgacacctgaggacactgccgtctattactgtactacagatggtaactacgtgaagggctactggggccaaggcaccactctcacagtctcctca

SEQ ID NO: 216 - skipped

SEQ ID NO: 217 - DNA, 351 bp, Mus musculus
  misc_feature: SC37.217  Heavy Chain Variable Region
gagattcagctgcagcagtctggggctgagcttgtgaggccaggggcctcagtcaagttgtcctgcacagcttctggctttaacattcaagccgactatatgcactgggtgaaacagaggcctgaacagggcctggagtggattggatggattgatcctgagattggtggtactgaatatgcctcgaagttccagggcaaggccactataacagcagacacatcctccaacacagtctacctgcagctcagcagcctgacatctgaggacactgccgtctattactgtattacagatggtaactacgtgaagggctactggggccaaggcaccactctcacagtctcctca

SEQ ID NO: 218 - protein, 117 aa, Mus musculus
  misc_feature: SC37.217 Heavy Chain Variable Region
EIQLQQSGAELVRPGASVKLSCTASGFNIQADYMHWVKQRPEQGLEWIGWIDPEIGGTEYASKFQGKATITADTSSNTVYLQLSSLTSEDTAVYYCITDGNYVKGYWGQGTTLTVSS

SEQ ID NO: 219 - DNA, 321 bp, Mus musculus
  misc_feature: SC37.223  Light Chain Variable Region
gatatccagatgacacagactacatcctccctgtctgcctctctgggagacagagtcaccatcagttgcagtgcaagtcagggcattggcaattatttaaactggtatcagcagaaaccagatggaactgttaaactcctgatctattacacatcaagtttaaattcaggagtcccatcaaggttcagtggcagtgggtctgggacagattattctctcaccatcagcaacctggaacctgaagatattgccacttatttttgtcagcagtatagtaagcttccgtacacattcggaggggggaccaagctggaaataaaa

SEQ ID NO: 220 - protein, 108 aa, Mus musculus
  misc_feature: SC37.223 Light Chain Variable Region
EDIQMTQTTSSLSASLGDRVTISCSASQGIGNYLNWYQQKPDGTVKLLIYYTSSLNSGVPSRFSGSGSGTDYSLTISNLEPEDIATYFCQQYSKLPYTFGGGTKLEIK

SEQ ID NO: 221 - DNA, 336 bp, Mus musculus
  misc_feature: SC37.223  Heavy Chain Variable Region
caggtccaactgcagcagcctggggctgaactggtgaagcctggggcttcagtgaagttgtcctgcaaggcttctggctacaccttcaccacctactatatttactgggtgaaacagaggcctggacaaggccttgagtggattggggggattaatcctagaaatggtggtactaacttcaatgagaaattcaagaccagggccacgctgactgtagacaaatcctccagcacagcctacatgcaactcagcagcctgacatctgaagactctgcggtctattactgtacaagaaccttctactggggccaaggcaccactctcacagtctcctca

SEQ ID NO: 222 - protein, 112 aa, Mus musculus
  misc_feature: SC37.223 Heavy Chain Variable Region
QVQLQQPGAELVKPGASVKLSCKASGYTFTTYYIYWVKQRPGQGLEWIGGINPRNGGTNFNEKFKTRATLTVDKSSSTAYMQLSSLTSEDSAVYYCTRTFYWGQGTTLTVSS

SEQ ID NO: 223 - DNA, 336 bp, Mus musculus
  misc_feature: SC37.226 Light Chain Variable Region
gatattgtgatgactcaggctgcaccctctgtacctgtcactcctggagagtcagtatccatctcctgcaggtctagtaagagtctcctgcatagtaatggcaacacttacttatattggttcctgcagaggccaggccagtctcctcaggtcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggctcggggacaaagttggaaataaaa

SEQ ID NO: 224 - protein, 112 aa, Mus musculus
  misc_feature: SC37.226 Light Chain Variable Region
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLEYPFTFGSGTKLEIK

SEQ ID NO: 225 - DNA, 345 bp, Mus musculus
  misc_feature: SC37.226  Heavy Chain Variable Region
gaggttcagctgcagcagtctggggctgagcttgtgaggccaggggcctcagtcaagttgtcctgcacagcttctggctttaacattatagtcgactatttgcactgggtgagacagaggcctgaacagggcctggagtggattggatggattgatcctgagattggttctactgaatatgcctcgaagttccagggcaaggccactatgacagcagacacatcctccaacacagcctacctgcagctcagcagcctgacatctgaggacactgccgtctattactgtattatcgacgggactatggactactggggtcaaggaacctcagtcaccgtctcctca

SEQ ID NO: 226 - protein, 115 aa, Mus musculus
  misc_feature: SC37.226 Heavy Chain Variable Region
EVQLQQSGAELVRPGASVKLSCTASGFNIIVDYLHWVRQRPEQGLEWIGWIDPEIGSTEYASKFQGKATMTADTSSNTAYLQLSSLTSEDTAVYYCIIDGTMDYWGQGTSVTVSS

SEQ ID NO: 227 - DNA, 321 bp, Mus musculus
  misc_feature: SC37.231 Light Chain Variable Region
gacattgtgatgacccagtctcaaaaattcatgtccacatcagtgggagacagggtcagcatcacctgcaaggccagtcagagtgttcgtcctgctgtagcctggtatcaacagaaaccagggcagtctcctaaagcactgatttacttggcatccaaccggcacactggagtccctgatcgcttcacaggcagtggatctgggacagatttcactctcaccattagcaatgtgcaatctgaagacctggcagattatttctgtctgcaacattggaattatccgtacacgttcggaggggggaccaagctggaaataaaa

SEQ ID NO: 228 - protein, 107 aa, Mus musculus
  misc_feature: SC37.231 Light Chain Variable Region
DIVMTQSQKFMSTSVGDRVSITCKASQSVRPAVAWYQQKPGQSPKALIYLASNRHTGVPDRFTGSGSGTDFTLTISNVQSEDLADYFCLQHWNYPYTFGGGTKLEIK

SEQ ID NO: 229 - DNA, 351 bp, Mus musculus
  misc_feature: SC37.231 Heavy Chain Variable Region
gaggttcagctgcagcagtctggggctgagcttgtgaggccaggggcctcagtcaagttgtcctgcacagcttctggctttaacattaaagacgactatatacactgggtgaagcagaggcctgaacagggcctggagtggattggatggattgaccctgagaatggtgatactaaatatgcctcgaagttcccgggcaaggccactatgacagcagacacatcctccaacacagcctacctgcagctcagcagcctgacatctgaggacactgccgtctattactgtactgcatccaggactacggcccttgactactggggcccaggcaccactctcacagtctcctca

SEQ ID NO: 230 - protein, 117 aa, Mus musculus
  misc_feature: SC37.231 Heavy Chain Variable Region
EVQLQQSGAELVRPGASVKLSCTASGFNIKDDYIHWVKQRPEQGLEWIGWIDPENGDTKYASKFPGKATMTADTSSNTAYLQLSSLTSEDTAVYYCTASRTTALDYWGPGTTLTVSS

SEQ ID NO: 231 - DNA, 351 bp, Mus musculus
  misc_feature: SC37.232  Heavy Chain Variable Region
gaggttcagctgcagcagtctggggctgagcttgtgaggccaggggcctcagtcaagttgtcctgcacagcttctggctttaacattgaggccgactatatgcactgggtgaagcagagccctgaacagggcctggagtggattggatggattgatcctgagattggtgctactgaatatgcctcgaagttccagggcaaggccactatgacagcagacacatcctccaacacagcctacctgcagctcagcagcctgacatctgaggacactggcgtctattactgtattacagatggtaactacgtgaagggctactggggccaaggcaccactctcacagtctcctca

SEQ ID NO: 232 - protein, 117 aa, Mus musculus
  misc_feature: SC37.232 Heavy Chain Variable Region
EVQLQQSGAELVRPGASVKLSCTASGFNIEADYMHWVKQSPEQGLEWIGWIDPEIGATEYASKFQGKATMTADTSSNTAYLQLSSLTSEDTGVYYCITDGNYVKGYWGQGTTLTVSS

SEQ ID NO: 233 - DNA, 321 bp, Mus musculus
  misc_feature: SC37.233  Light Chain Variable Region
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcgaggttcagtggcagtgggtctgggacagattattctctcaccatcagcaacctggaacctgaagatattgccacttactattgtcagcagtatagaaagcttccgtacacgttcggaggggggaccaagctggaaataaaa

SEQ ID NO: 234 - protein, 107 aa, Mus musculus
  misc_feature: SC37.233 Light Chain Variable Region
DIQMTQTTSSLSASLGDRVTISCSASQGISNYLNWYQQKPDGTVKLLIYYTSSLHSGVPSRFSGSGSGTDYSLTISNLEPEDIATYYCQQYRKLPYTFGGGTKLEIK

SEQ ID NO: 235 - DNA, 336 bp, Mus musculus
  misc_feature: SC37.233 Heavy Chain Variable Region
caggtccaactgcagcagcctggggctgaactggtgaagcctggggcttcagtgaagttgtcctgcaaggcttctggctacaccttcaccacctactatatttactgggtgaaacagaggcctggacaaggccttgagtggattggggggattaatcctagaaatggtggtactaacttcaatgagaaattcatgagcagggccacactgactgtagacaaatcctccagcacagcctacatgcaactcagcagcctgacatctgaggactctgcggtctattactgttcaagaagcttctactggggccaaggcaccactctcattgtctcctca

SEQ ID NO: 236 - protein, 112 aa, Mus musculus
  misc_feature: SC37.233 Heavy Chain Variable Region
QVQLQQPGAELVKPGASVKLSCKASGYTFTTYYIYWVKQRPGQGLEWIGGINPRNGGTNFNEKFMSRATLTVDKSSSTAYMQLSSLTSEDSAVYYCSRSFYWGQGTTLIVSS

SEQ ID NO: 237 - DNA, 336 bp, Mus musculus
  misc_feature: SC37.236  Light Chain Variable Region
gacattgtgatgtcacagtctccatcgtccctagctgtgtcagttggagagaaggttactatgagctgcaagtccagtcagagccttttatatagtagaaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctattggacgttcggtggaggcaccaagctggaaatcaaa

SEQ ID NO: 238 - protein, 112 aa, Mus musculus
  misc_feature: SC37.236 Light Chain Variable Region
DIVMSQSPSSLAVSVGEKVTMSCKSSQSLLYSRNQKNYLAWYQQKPGQSPKLLIYWASTRESGVPDRFTGSGSGTDFTLTISSVKAEDLAVYYCQQYYSYWTFGGGTKLEIK

SEQ ID NO: 239 - DNA, 363 bp, Mus musculus
  misc_feature: SC37.236  Heavy Chain Variable Region
caggtccaactgcagcagcctggggctgagctggtgaggcctgggtcttcagtgaagctgtcctgcaaggcttctggctacaccttcaccacctactggatggattgggtgaagcagaggcctggacaaggccttgaatggattggtaacatttacccttctgatagtgaaactcactacaatccaaagttcaaggacaaggccacattgactgtagacatatcctccagcacagcctacatgcagctcagcagcctgacatctgaggactctgcggtctattactgtgcacgacattactacgctggtagcccgttctactttgactactggggccaaggcacctctctcacagtctcctca

SEQ ID NO: 240 - protein, 121 aa, Mus musculus
  misc_feature: SC37.236 Heavy Chain Variable Region
QVQLQQPGAELVRPGSSVKLSCKASGYTFTTYWMDWVKQRPGQGLEWIGNIYPSDSETHYNPKFKDKATLTVDISSSTAYMQLSSLTSEDSAVYYCARHYYAGSPFYFDYWGQGTSLTVSS

SEQ ID NO: 241 - DNA, 336 bp, Mus musculus
  misc_feature: SC37.239  Light Chain Variable Region
gacattgtgatgtcacagtctccatcctccctagctgtgtcagttggagagaaggttactatgagctgcaagtccagtcagagccttttatatagtaccaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataactatctcacgttcggtgctgggaccaagctggagctgaaa

SEQ ID NO: 242 - protein, 112 aa, Mus musculus
  misc_feature: SC37.239 Light Chain Variable Region
DIVMSQSPSSLAVSVGEKVTMSCKSSQSLLYSTNQKNYLAWYQQKPGQSPKLLIYWASTRESGVPDRFTGSGSGTDFTLTISSVKAEDLAVYYCQQYYNYLTFGAGTKLELK

SEQ ID NO: 243 - DNA, 360 bp, Mus musculus
  misc_feature: SC37.239  Heavy Chain Variable Region
caggtttctctgaaagagtcgggccctgggatattgcagccctcccagaccctcagtctgacttgttctttctctgggttttcactgagcagttctggtatggctgtaggctggattcgtcagccttcagggaagggtctggagtggctggcaaacatttggtgggatgatgataagcactataacgcagccctgaagagccggctcacaatctccaaggatacctccaaaaaccaggtattcctcaagatcgccagtgtggacactgcagatactgccacatactactgtgctcgaacgggtgacttgcgggcctactttgactactggggccaaggcaccactctcacagtctcctca

SEQ ID NO: 244 - protein, 120 aa, Mus musculus
  misc_feature: SC37.239 Heavy Chain Variable Region
QVSLKESGPGILQPSQTLSLTCSFSGFSLSSSGMAVGWIRQPSGKGLEWLANIWWDDDKHYNAALKSRLTISKDTSKNQVFLKIASVDTADTATYYCARTGDLRAYFDYWGQGTTLTVSS

SEQ ID NO: 245 - DNA, 336 bp, Mus musculus
  misc_feature: SC37.243  Light Chain Variable Region
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcacgatcagcagagtggaggctgaggatctgggagtttattactgctttcaaggttcacatgttccgctcacgttcggtgctgggaccaagctggagctgaaa

SEQ ID NO: 246 - protein, 112 aa, Mus musculus
  misc_feature: SC37.243 Light Chain Variable Region
DVLMTQTPLSLPVSLGDQASISCRSSQSIVHSNGNTYLEWYLQKPGQSPKLLIYKVSNRFSGVPDRFSGSGSGTDFTLTISRVEAEDLGVYYCFQGSHVPLTFGAGTKLELK

SEQ ID NO: 247 - DNA, 342 bp, Mus musculus
  misc_feature: SC37.243  Heavy Chain Variable Region
gatgtgcagcttcaggagtcaggacctgacctggtgaaaccttctcagtcactttcactcacctgcactgtcactggctactccatcaccagtggtcttggctggcactggatccggcagtttccaggatacaaactggagtggatgggctacataagctacagtggtagtaataactacaatccatctctcaaaagtcgaatctctatcactcgagacacatccaagaaccagttcttcctgcagttgaattctgtgactaatgaggacacagccacatattactgtgcaagccatggtgacggtgtctggggcgcagggaccacggtcaccgtctcctca

SEQ ID NO: 248 - protein, 114 aa, Mus musculus
  misc_feature: SC37.243 Heavy Chain Variable Region
DVQLQESGPDLVKPSQSLSLTCTVTGYSITSGLGWHWIRQFPGYKLEWMGYISYSGSNNYNPSLKSRISITRDTSKNQFFLQLNSVTNEDTATYYCASHGDGVWGAGTTVTVSS

SEQ ID NO: 249 - DNA, 336 bp, Mus musculus
  misc_feature: SC37.244  Light Chain Variable Region
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 250 - protein, 112 aa, Mus musculus
  misc_feature: SC37.244 Light Chain Variable Region
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 251 - DNA, 360 bp, Mus musculus
  misc_feature: SC37.244  Heavy Chain Variable Region
caggttcaactgcagcagtctggagctgagctggcgaggcctggggcttcagtgaagctgtcctgcaaggcttctggctacaccttcacaagctatggtataagctgggtgaagcagagaactggacagggccttgagtggattggagagatttttcctagaactggtaatacttactacaatgagaagttcaagggcaaggccacactgactgcagacaaatcctccagcacagcgtacatggagctccgcagcctgacatctgaggactctgcggtctatttctgtgcaagaggggattactacggtagtagctcctttgactactggggccaaggcaccactctcacagtctcctca

SEQ ID NO: 252 - protein, 120 aa, Mus musculus
  misc_feature: SC37.244 Heavy Chain Variable Region
QVQLQQSGAELARPGASVKLSCKASGYTFTSYGISWVKQRTGQGLEWIGEIFPRTGNTYYNEKFKGKATLTADKSSSTAYMELRSLTSEDSAVYFCARGDYYGSSSFDYWGQGTTLTVSS

SEQ ID NO: 253 - DNA, 339 bp, Artificial Sequence
  hSC37.2 Light Chain Variable Region
gatatccagatgacccagtctccttctagccttagcgcctcagtgggcgaccgcgtgactatcacttgtaaatcctcccagacattgctgtattacagcaatcaaaaaaactatcttgcctggtaccaacagaagccaggtaaagtcccaaaactgctgatctactgggcctctaccagagacagcggtgtcccctcacggttttctgggtcaggaagtggaaccgacttcactctgacaatcagttccctgcagcctgaggacgtagctacttattactgtcagcagtactataactatccattgacttttgggcagggaaccaaactggaaatcaag

SEQ ID NO: 254 - protein, 113 aa, Artificial Sequence
  hSC37.2 Light Chain Variable Region
DIQMTQSPSSLSASVGDRVTITCKSSQTLLYYSNQKNYLAWYQQKPGKVPKLLIYWASTRDSGVPSRFSGSGSGTDFTLTISSLQPEDVATYYCQQYYNYPLTFGQGTKLEIK

SEQ ID NO: 255 - DNA, 360 bp, Artificial Sequence
  hSC37.2 Heavy Chain Variable Region
gaggtgcagttggttgagtccggggggggcctggtacagcctggaggaagccttagattgagctgtgcagcctctgggttcaccttttctgatttcgatatggagtgggtccggcaagcccccggcaaaggactggaatgggtcggtgcctccaggaataaggccaacgattacacaacccaatactccgcctcagtgaagggacgattcacaatctcacgggacacctctaaaaatagtctgtacttgcagatgaacagcctcaagacagaagatactgcagtctattattgcgctagagatcgttacgactatgccctcgattattggggccagggtaccaccgtaacagtctcatct

SEQ ID NO: 256 - protein, 120 aa, Artificial Sequence
  hSC37.2 Heavy Chain Variable Region
EVQLVESGGGLVQPGGSLRLSCAASGFTFSDFDMEWVRQAPGKGLEWVGASRNKANDYTTQYSASVKGRFTISRDTSKNSLYLQMNSLKTEDTAVYYCARDRYDYALDYWGQGTTVTVSS

SEQ ID NO: 257 - DNA, 333 bp, Artificial Sequence
  hSC37.17  Light Chain Variable Region
gatatcgtcatgacacagtcccctgacagcctggccgtgtccctgggcgaacgagctactatcaactgtcgtgcatctgaatccgtagactcttatggcaattcattcatgcactggtatcagcagaaacccggtcagccacccaaactgctgatatacctcgcttccaaccttgaaagcggagtgccagatcgcttctccgggagtgggtctggcactgacttcaccctgactatttccagcctccaggcagaggacgtggccgtatattactgtcagcagaacaacgaggaccctctcacctttgggcagggcaccaaggtagagatcaaa

SEQ ID NO: 258 - protein, 111 aa, Artificial Sequence
  hSC37.17 Light Chain Variable Region
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIK

SEQ ID NO: 259 - DNA, 354 bp, Artificial Sequence
  hSC37.17  Heavy Chain Variable Region
caagtgcagctggtgcagtctggcgccgaggtgaagaaaccaggcgcctctgtcaaggtgagttgcaaggcatccggttttaacatcaaggacacttatatccactgggtcaggcaagcacctggccagggattggaatggatgggccggattgatccagctaatggcaaggccaactacgaccctaaattccaggggcgtgttactatgacccgcgacaccagcacctccacagtctacatggagctcagttcattgcgaagcgaggatacagccgtgtattactgcgctttgggtggcggatactatggaatggactactggggacagggaaccctcgttactgtttcctca

SEQ ID NO: 260 - protein, 118 aa, Artificial Sequence
  hSC37.17 Heavy Chain Variable Region
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSS

SEQ ID NO: 261 - DNA, 321 bp, Artificial Sequence
  hSC37.39 Light Chain Variable Region
gaaatagtaatgacccaatctcccgccacattgtctgtgtcccctggagagcgggccaccctgagttgtaaggcatctcagaacgtaggcatcaatgtggcatggtaccagcagaaacccggccaggcccctcgagccctgatttactccgcaagctatcggtattccgggatacctgcacgcttttccgggagcggctctggcaccgagttcacacttacaatttcttccctccagagtgaggatttcgctgtttactattgccatcaatacaagacatatccctatacattcggaggcggcactaaggtggagatcaag

SEQ ID NO: 262 - protein, 107 aa, Artificial Sequence
  hSC37.39 Light Chain Variable Region
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKVEIK

SEQ ID NO: 263 - DNA, 339 bp, Artificial Sequence
  hSC37.39 Heavy Chain Variable Region
caggtgcagttggtgcagtctggggcagaagtgaagaagccaggagccagtgtaaaggtgtcttgcaaagcaagtggctacacattcaccacttataccatccactgggtaaggcaggctcccggacaaggtctcgaatggatggggtacatcaatcctaggtccggctataccgagtataatcagaaattccaagaccgtgtcactatgaccagggatacttcaacatccacagtctatatggaactgagctccctgagaagtgaggacaccgccgtgtattattgcgctaggtcttatgaattttggggacagggcaccactgttactgtatcaagc

SEQ ID NO: 264 - protein, 113 aa, Artificial Sequence
  hSC37.39 Heavy Chain Variable Region
QVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSS

SEQ ID NO: 265 - DNA, 321 bp, Artificial Sequence
  hSC37.67  Light Chain Variable Region
gacattcagatgactcagtctcctagctccttgtcagcctccgtcggtgatcgtgtgacaattacctgtaaagcctctgaggacatatataatagactggcctggtatcagcaaaagcctgggaaggccccaaagctgctgatttctggtgccacaagtctggagactggcgttccttccaggtttagcggaagtggaagtggcactgattataccctgacaatctccagcttgcagcccgaagactttgcaacctactactgtcagcagaattggtctactccccctacttttgggggcggcactaaggtcgagatcaag

SEQ ID NO: 266 - protein, 107 aa, Artificial Sequence
  hSC37.67 Light Chain Variable Region
DIQMTQSPSSLSASVGDRVTITCKASEDIYNRLAWYQQKPGKAPKLLISGATSLETGVPSRFSGSGSGTDYTLTISSLQPEDFATYYCQQNWSTPPTFGGGTKVEIK

SEQ ID NO: 267 - DNA, 339 bp, Artificial Sequence
  hSC37.67  Heavy Chain Variable Region
caggtgcaactcgtgcagagcggggcagaagttaaaaaaccaggggcctccgtaaaagttagttgcaaagcctcaggttacacattcacaaggtactggatagaatgggtcagacaggctcccggccagcgtctggaatggatgggcgagatactcccagggtctggttcaaccaactacaacgagaagttcaaggggcgcgtgactattaccgcggacacaagcgcctcaaccgcttacatggagctgagtagtctgcgaagcgaggatacagctgtctactactgtgagcgcaggggggcctactgggggcagggcactcttgtgacagtctcctct

SEQ ID NO: 268 - protein, 113 aa, Artificial Sequence
  hSC37.67 Heavy Chain Variable Region
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSS

SEQ ID NO: 269 - DNA, 321 bp, Artificial Sequence
  hSC37.67 variant 1 Light Chain Variable Region
gacattcagatgactcagtctcctagctccttgtcagcctccgtcggtgatcgtgtgacaattacctgtaaagcctctgaggacatatataatagactggcctggtatcagcaaaagcctgggaaggccccaaagctgctgatttctggtgccacaagtctggagactggcgttccttccaggtttagcggaagtggaagtggcactgattataccctgacaatctccagcttgcagcccgaagactttgcaacctactactgtcagcagcagtggtctactccccctacttttgggggcggcactaaggtcgagatcaag

SEQ ID NO: 270 - protein, 107 aa, Artificial Sequence
  hSC37.67 variant 1 Light Chain Variable Region
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIK

SEQ ID NO: 271 - protein, 220 aa, Artificial Sequence
  hSC37.2 Light Chain
DIQMTQSPSSLSASVGDRVTITCKSSQTLLYYSNQKNYLAWYQQKPGKVPKLLIYWASTRDSGVPSRFSGSGSGTDFTLTISSLQPEDVATYYCQQYYNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 449 aa, Artificial Sequence
  hSC37.2 Heavy Chain
EVQLVESGGGLVQPGGSLRLSCAASGFTFSDFDMEWVRQAPGKGLEWVGASRNKANDYTTQYSASVKGRFTISRDTSKNSLYLQMNSLKTEDTAVYYCARDRYDYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 218 aa, Artificial Sequence
  hSC37.17 Light Chain
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 447 aa, Artificial Sequence
  hSC37.17 Heavy Chain
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - protein, 447 aa, Artificial Sequence
  hSC37.17ss1 Heavy Chain
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 276 - protein, 214 aa, Artificial Sequence
  hSC37.39 Light Chain
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 442 aa, Artificial Sequence
  hSC37.39 Heavy Chain
QVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 278 - protein, 442 aa, Artificial Sequence
  hSC37.39.ss1 Heavy Chain
QVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 279 - protein, 214 aa, Artificial Sequence
  hSC37.67 Light Chain
DIQMTQSPSSLSASVGDRVTITCKASEDIYNRLAWYQQKPGKAPKLLISGATSLETGVPSRFSGSGSGTDYTLTISSLQPEDFATYYCQQNWSTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 442 aa, Artificial Sequence
  hSC37.67 Heavy Chain
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 281 - protein, 214 aa, Artificial Sequence
  hSC37.67 variant 1 Light Chain
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 17 aa, Mus musculus
  misc_feature: hSC37.2 CDRL1
KSSQTLLYYSNQKNYLA

SEQ ID NO: 283 - protein, 7 aa, Mus musculus
  misc_feature: hSC37.2 CDRL2
WASTRDS

SEQ ID NO: 284 - protein, 9 aa, Mus musculus
  misc_feature: hSC37.2 CDRL3
QQYYNYPLT

SEQ ID NO: 285 - protein, 5 aa, Mus musculus
  misc_feature: hSC37.2 CDRH1
DFDME

SEQ ID NO: 286 - protein, 19 aa, Mus musculus
  misc_feature: hSC37.2 CDRH2
ASRNKANDYTTQYSASVKG

SEQ ID NO: 287 - protein, 9 aa, Mus musculus
  misc_feature: hSC37.2 CDRH3
DRYDYALDY

SEQ ID NO: 288 - protein, 15 aa, Mus musculus
  misc_feature: hSC37.17 CDRL1
RASESVDSYGNSFMH

SEQ ID NO: 289 - protein, 7 aa, Mus musculus
  misc_feature: hSC37.17 CDRL2
LASNLES

SEQ ID NO: 290 - protein, 8 aa, Mus musculus
  misc_feature: hSC37.17 CDRL3
QQNNEDPL

SEQ ID NO: 291 - protein, 5 aa, Mus musculus
  misc_feature: hSC37.17 CDRH1
DTYIH

SEQ ID NO: 292 - protein, 17 aa, Mus musculus
  misc_feature: hSC37.17 CDRH2
RIDPANGKANYDPKFQG

SEQ ID NO: 293 - protein, 9 aa, Mus musculus
  misc_feature: hSC37.17 CDRH3
GGGYYGMDY

SEQ ID NO: 294 - protein, 11 aa, Mus musculus
  misc_feature: hSC37.39 CDRL1
KASQNVGINVA

SEQ ID NO: 295 - protein, 7 aa, Mus musculus
  misc_feature: hSC37.39 CDRL2
SASYRYS

SEQ ID NO: 296 - protein, 9 aa, Mus musculus
  misc_feature: hSC37.39 CDRL3
HQYKTYPYT

SEQ ID NO: 297 - protein, 5 aa, Mus musculus
  misc_feature: hSC37.39 CDRH1
TYTIH

SEQ ID NO: 298 - protein, 17 aa, Mus musculus
  misc_feature: hSC37.39 CDRH2
YINPRSGYTEYNQKFQD

SEQ ID NO: 299 - protein, 4 aa, Mus musculus
  misc_feature: hSC37.39 CDRH3
SYEF

SEQ ID NO: 300 - protein, 11 aa, Mus musculus
  misc_feature: hSC37.67 CDRL1
KASEDIYNRLA

SEQ ID NO: 301 - protein, 7 aa, Mus musculus
  misc_feature: hSC37.67 CDRL2
GATSLET

SEQ ID NO: 302 - protein, 9 aa, Mus musculus
  misc_feature: hSC37.67 CDRL3
QQNWSTPPT

SEQ ID NO: 303 - protein, 5 aa, Mus musculus
  misc_feature: hSC37.67 CDRH1
RYWIE

SEQ ID NO: 304 - protein, 17 aa, Mus musculus
  misc_feature: hSC37.67 CDRH2
EILPGSGSTNYNEKFKG

SEQ ID NO: 305 - protein, 4 aa, Mus musculus
  misc_feature: hSC37.67 CDRH3
RGAY

SEQ ID NO: 306 - protein, 9 aa, Mus musculus
  misc_feature: hSC37.67v1 CDRL3
QQQWSTPPT